(19) United States
(12) Patent Application Publication
(10) Pub. No.: US 2023/0201550 A1
(43) Pub. Date: Jun. 29, 2023

(54) SYSTEMS AND METHODS FOR TREATING PERSISTENT PAIN OF NEUROGENIC ORIGIN AND COMPLEX INJURY

(71) Applicant: Nova Neura, LLC, Phoenix, AZ (US)

(72) Inventor: David George, Phoenix, AZ (US)

(21) Appl. No.: 18/001,063

(22) PCT Filed: Jun. 4, 2021
(86) PCT No.: PCT/US2021/035998

§ 371 Date: Dec. 7, 2022

Related U.S. Application Data
(60) Provisional application No. 63/036,173, filed on Jun. 8, 2020.

(51) Int. Cl. A61M 37/00; A61K 31/045; A61K 31/167; A61K 9/00; A61M 19/00; A61N 1/36

(52) U.S. Cl. CPC A61M 37/00; A61K 31/045; A61K 31/167; A61K 9/0014; A61M 19/00; A61N 1/36021; A61M 2021/0066

(57) ABSTRACT

An applicator can include a dispenser for dispensing an amount of a therapeutic composition, which can include an amount of sugar or sugar alcohol and an amount of an alkalizing agent. In some cases, the alkalizing agent can include L-Arginine. In some cases, 75% or more of the composition by weight has a comedogenicity rating of 3 or less on a scale of 0-5. The dispenser can also have a power supply configured to provide power through a contact tip of the dispenser to provide nerve stimulation, such as using vibration, heating, cooling, and/or electrical stimulation.

### CROSS-REFERENCE TO RELATED APPLICATIONS

This application claims the benefit under 35 U.S.C. § 119(e) of U.S. Provisional Patent Application No. 63/036,173, filed Jun. 8, 2020, and titled SYSTEMS AND METHODS FOR TREATING PERSISTENT PAIN OF NEUROGENIC ORIGIN AND COMPLEX INJURY. The entirety contents of each of the above-identified application(s) are hereby incorporated by reference herein and made part of this specification for all that they disclose.

### INCORPORATION BY REFERENCE

The entirety contents of each of the following references are hereby incorporated by reference herein and made part of this specification for all that they disclose: U.S. Patent Application Publication No. 2013/0236577, published Sep. 12, 2013, and titled PAIN RELIEVER COMPOSITION; U.S. Patent Application Publication No. 2015/0148429, published May 28, 2015, and titled PAIN RELIEVING SYSTEM; and U.S. Patent Application Publication No. 2017/0360867, published Dec. 21, 2017, and titled PAIN RELIEVING SYSTEM.

### BACKGROUND

### Field of the Disclosure

Some embodiments disclosed herein relate generally to physical therapeutic devices and methods, including topical preparations for pain management, manual therapy methods for pain and injury rehabilitation, and neuromodulation for rehabilitating the human nervous system.

### SUMMARY OF CERTAIN EMBODIMENTS

Certain example embodiments are summarized below for illustrative purposes. The embodiments are not limited to the specific implementations recited herein. Embodiments may include several novel features, no single one of which is solely responsible for its desirable attributes or which is essential to the embodiments.

Particular embodiments of the subject matter described in this specification can be implemented so as to realize one or more of the following advantages: A therapeutic system is described that can safely alleviate severity, frequency or duration of pain associated with nerve irritation, central nervous system dysfunction, musculoskeletal injury or degeneration, compromise to muscular structures, connective tissues, dermatologic tissues and fascia. This integrative system can also be used to alleviate symptoms associated with surgery whilst facilitating post-injury or post-surgical tissue regeneration, recovery and rehabilitation. In consideration of the latter objective, the therapeutic approach can also be utilized to aid in recovery of muscle strength or to promote muscle function. This system involves a series of components that can be utilized individually or as a complete system.

As will be discussed, a component of this system is an approach to therapeutic topical development, production and application that improves the efficacy of therapeutic agents by creating a conducive metabolic environment in target tissue that improves the response to a given therapeutic agent or combination of therapeutic agents. Furthermore, the method facilitates the delivery of therapeutic compounds through the skin, optimizing the volume of active ingredients arriving at target tissues beneath the skin, in a safe and comfortable manner. As a third component, this therapeutic method also optimizes the benefits of physically-based therapies (e.g. physical therapy, massage, chiropractic, orthopedics and physical medicine) by providing a means to mitigate the range of adverse factors that complicate recovery and rehabilitation posed by nerve irritation. Each of the above elements also offers benefits in optimization of bodily functions in the absence of injury, including in the pursuit of optimal tissue status of the dermatologic, reproductive, respiratory, cardiovascular, digestive and dental tissues.

As will be described, this therapeutic system can include the use of a device that can receive information from a sensor at or close to the users body as well as through a user device (e.g., a mobile device, such as a smart phone, a wearable device, a tablet, a laptop, a computer) being operated by a patient, with the information specifying treatment parameter profiles to utilize to reduce perceived pain by the patient. Through use of the user device, the patient can indicate pain he/she is experiencing (e.g., particular symptoms, particular body areas being affected by one or more symptoms, intensity levels of the pain, and so on as will be described), and indicate whether applied treatment parameter profiles are effective. The system will monitor responses from the patient (e.g., patient specified or indicated information, or optionally physiological information from the patient, for instance a heart rate as determined by a device, such as a smartwatch or fitness tracker, worn by the patient), the user device can determine treatment parameter profiles that best reduce or alleviate pain symptoms. For example, the patient can specify particular symptoms the patient is experiencing, and the user device can determine a treatment parameter profile that best reduces the particular symptoms. The device will also monitor muscular exertion on the part of the patient to uniquely adapt to that feedback by generally speaking, raising, lowering or modifying output or delivery of the therapeutic stimulus or agent to the patient. The device will also offer the user feedback to facilitate the recovery process.

This system involves a number of approaches to the treatment of physical conditions that have been significant medical challenges for some time. These include but are not limited to Tinnitus, Peripheral Neuropathy (incl. poly- and mono-neuropathies), Headache Symptoms and Conditions (incl. Migraine), Pain Conditions (incl. Lower Back Pain and Patellofemoral Pain), post-surgical pain, complex regional pain syndromes, chronic fatigue, and generally impaired physical performance.

This system also involves an approach to treating peripheral neurogenic symptoms (e.g. associated with various forms of peripheral neuropathy), which involves the simultaneous application of a novel transdermal compound to the affected peripheral nerves, whilst simultaneously, or in sequence, applying a form of stimulus to affect plasticity in the spinal cord and brain. Whilst various forms of sensory stimulation have been reported in the literature for affecting sensory symptoms, this approach is particularly beneficial because the motor system is utilized as a means to modify the sensory system (e.g., muscles are exogenously activated beyond normal activation patterns to create changes in the spinal cord and brain, which affects the perception of symptoms associated with neurogenic inflammation and peripheral neuropathy). A particular combination of stimulation superimposed with voluntary muscle recruitment is disclosed.

The details of one or more embodiments of the subject matter of this specification are set forth in the accompanying drawings and the description below. Other features, aspects, and advantages of the subject matter will become apparent from the description, the drawings, and from the claims.

### BRIEF DESCRIPTION OF THE DRAWINGS

Certain embodiments will be discussed in detail with reference to the following figures, wherein like reference numerals refer to similar features throughout. These figures are provided for illustrative purposes and the embodiments are not limited to the specific implementations illustrated in the figures.

FIG. 1 shows a plot illustrating a first concentration response to a topical therapeutic.

FIG. 2 shows a plot illustrating a second concentration response to a topical therapeutic.

FIG. 3 shows an example embodiment of an applicator device.

FIG. 4 is a cross-sectional view of an example embodiment of an applicator device.

FIG. 5 is a cross-sectional view of an example embodiment of an applicator device.

FIG. 6 is a cross-sectional view of an example embodiment of an applicator device.

FIG. 7 is a cross-sectional view of an example embodiment of an applicator device.

FIG. 8 is a cross-sectional view of an example embodiment of an applicator device.

FIG. 9 is a cross-sectional view of an example embodiment of an applicator device.

FIG. 10 shows an example embodiment of an applicator device.

FIG. 11 shows an example embodiment of an applicator device.

FIG. 12 is a cross-sectional view of an example embodiment of an applicator device.

FIG. 13 shows potential excitatory/inhibitory and excitatory/excitatory configurations for method of autonomic modulation via topical compounds.

FIG. 14 shows an example method of manipulating a therapeutic skin device interface. The interface makes contact with skin surface. The interface can be manipulated by hand or device via anchor points on its outer surface. Superficial nerves can be addressed mechanically by movement of the interface and chemically by a therapeutic compound for compound effect.

FIG. 15 shows example forms of adhesive therapeutic interface as applied to superficial nerves of the head and neck, addressing supraorbital, supratrochlear, infraorbital, infratrochlear, anterior ethmoidal, lesser occipital, greater occipital, Erb's point, anterior-, posterior- and lateral supraclavicular nerves.

FIG. 16 shows example forms of adhesive therapeutic interface as applied to superficial nerves of the lower back and hip, addressing superior-, middle (medial)-, inferior cluneal, iliohypogastric, and subcostal nerves. Application a. can be particularly effective for lower back pain (e.g., treating combined gluteal and paraspinal sites to address superficial nerves, as opposed to other applications that focus on paraspinal sites only). Application b. can be effective for lower back and groin pain. Application c. can be effective for lower back pain and hip pain.

FIG. 17 shows example forms of adhesive therapeutic interface as applied to superficial nerves of the lower limbs and feet. Application a. can be beneficial for knee conditions, such as by covering femoral, lateral femoral cutaneous and saphenous nerves. Application b can be beneficial for foot and ankle conditions, such as by addressing tibial and peroneal nerves at the popliteal fossa, sural, superficial peroneal and posterior tibial, and plantar nerves. Application c. can be beneficial for further applications for the foot and ankle including interdigital and plantar nerves.

FIG. 18 shows an example embodiment of a therapeutic device a) & b): valves preventing transmission of therapeutic compound from container section into dispensing channel. These valves can be actuated by simple, manually-induced pressure (e.g., from deformation of the container walls) or by automation wherein the valve and dispensing mechanism is actuated by an automated mechanism. c): channel and potential valve mechanism serving as final common pathway for dispensing therapeutic compound. d) & e): containers storing one or more therapeutic compounds. f): rolling cylinder. g): battery supplying power to automated components. h): interface platform, which may be a solid component penetrated by a channel to convey therapeutic compounds from storage compartments to the interface with the body, or, may house components of energy-generating system (including electrical, light, thermal, or kinetic energy). The interface component f) can take multiple forms including: fi) one or more rolling spheres; fii) single rolling or static sphere; fiii) solid surface penetrated by fluid channel; fiv) textured surface to enhance grip with target surface. fv) shows an example of an energy-activating component wherein depression of the interface component activates an energy-generating component of the system. 1) is an interface with the skin surface (which may be a conductor of thermal, electrical, kinetic or other energy form). 2) is a spring or reformable depression-resisting component. 3) is a circuit trigger. 4) is a portion of circuit connecting trigger with energy-generating system and potential pathway for electrical energy transmitted to the interface component. 5) is a potential valve controlling dispensing of therapeutic agent. 6) is a channel to a storage compartment.

FIG. 19 shows an example application method of therapeutic device system with positive or negative pressure. In example embodiment (a), positive pressure is exerted via the device on superficial tissues in order to contact the underlying superficial nerve. In some embodiments, small movements are performed or delivered with the interface contact on the skin so as to mechanically liberate the underlying nerve. In example embodiment (b), a negative (e.g., traction) pressure is exerted on the skin surface (e.g., secondarily transmitting this force to underlying tissues, including the superficial nerve). In some embodiments, small movements are transmitted to the arrested tissue, serving to dislodge or liberate underlying nerves. In the aforementioned embodiments, the device system can deliver a topical compound that synergistically acts to improve the therapeutic effect, and in some cases also assists in the transmission of a therapeutic energy (e.g. electricity or light). The topical analgesic can act to mitigate pain and inflammation of the target nerve, which can reduce edema and counteract the friction forces serving to entrap the nerve. The example embodiment (c) shows an example application of the device system at the superficial nerves of the face, wherein target nerves are addressed with a combination of therapeutic agents (e.g., topical agent delivered in concert with kinetic and/or electric energy).

FIG. 20 shows a device for stimulating motor and sensory pathways of the central nervous system (CNS). a) is a sensor component of system, which is activated by i) stretch/deformation, ii) movement of two or more points from or toward each other (e.g., by transducing stretch or relative distance into an electrical signal). b) is a resistance band or material. c) is a transmitter component (which can be wireless or hardwire), connecting the sensor component to the controller portion of the device system. d) is a controller portion. e) is on output of the controller for sending electrical signals. The controller can be capable of powering and generating electrical signals based on input from the sensor. The output signals can be carried by hardwire (f) or wirelessly. g) are electrodes placed on target muscle groups. In an embodiment of this device the electrodes would be placed to target the gluteal muscles. h) are additional electrodes (in another embodiment) targeting the paraspinal area to produce a second pattern of contracting stimuli. In an example embodiment the system can generate cyclical, pulsing, contractions of the paraspinals, e.g., either simultaneously or alternating.

FIG. 21 shows a block diagram of an example embodiment of a system for stimulating motor and sensory pathways of CNS through multiple simultaneous and synergistic therapeutic processes. Each of these processes are described in various embodiments in this application. Each of these processes are delivered by the various therapeutic device embodiments described in this application.

FIG. 22 shows therapeutic interfaces applied for mononeuropathies. a) can be for carpal tunnel syndrome (e.g., median neuropathy at the wrist). The crosshatched portion demonstrates one, shorter embodiment, while the single hatched area shows an extended embodiment, which covers the median nerve at the elbow. b) can be fore ulnar neuropathy at the elbow (e.g., cubital tunnel syndrome). The crosshatched area shows a smaller embodiment, while the single hatched area shows an extended embodiment covering the full length of the ulnar nerve to the wrist. c) can be for lower limb applications for achilles tendonitis, peripheral polyneuropathy (including diabetic polyneuropathy), tarsal tunnel syndrome and plantar-fasciitis and -neuropathy; sites include popliteal fossa, bilaterally at the Achilles tendon, over the tarsal tunnel (medial foot), medial aspect of calf, medial and lateral aspect of the foot (not pictured: dorsal foot between metatarsals). d) can be for peroneal neuropathy—at the knee and/or—at the ankle (coverage including popliteal fossa, lateral knee (over pathway of peroneal nerve at fibula), anterior ankle (dorsal foot), and between great and second toe.

FIG. 23 shows a flowchart of an example embodiment of a device-assisted method for treatment of neurogenic pain and conditions associated with neurogenic provocation, involvement and origin.

FIG. 24 shows an example embodiment of a negative pressure, multi-energy platform dispenser. FIG. 24 shows the dispenser and body interface surface. A housing containing at least one chamber with a composition is not shown in FIG. 24.

FIG. 25 shows the interior of the example embodiment of FIG. 24.

FIG. 26 shows the example embodiment of FIG. 24, also with the exterior omitted from view to show internal elements. A the contact surface can have a rigid ring, which can be pressed against tissue. One or more electrodes (e.g., an anode and cathode) can be positioned on the ring so as to contact the tissue for electrical stimulation. Fluid delivery pathways can deliver a topical therapeutic composition to opening in the contact interface, as described herein. In the center, a pressure cup can apply negative pressure, such as via suction applied through a center fluid pathway, using a manual pump (e.g., a squeezable bulb) or an electronic pump.

FIG. 27 shows an example embodiment of a negative pressure, multi-energy platform device component. The dispenser component is shown, and can be used with a housing that has a compartment with a topic composition, as described herein.

FIG. 28 shows a side view of the component of FIG. 27.

FIG. 29 shows another view of the component of FIG. 27. The component can have a contact interface (e.g., in the shape of a ring). The contact interface can have one or more electrodes (e.g., anode and cathode), and a hollow space in the center to generate negative pressure, and side channels for therapeutic agent.

FIG. 30 shows an example embodiment of a component of a negative pressure therapy device. The contact surface is shown. Negative pressure can be applied via suction through a fluid channel (e.g., using a manual pump like a suction bulb or an electronic pump). The contact surface can have two openings, which can receive the suction from the fluid channel, so that tissue can be pulled by both opening. Between the openings is a ridge or dividing structure. The dividing structure can press against the tissue between the two tissue areas that are being pulled by the two openings.

FIG. 31 is another view of the component of FIG. 30.

FIG. 32 is another view of the component of FIG. 30.

FIG. 33 shows another example embodiment of a component of a therapy device, which can have two openings similar to the embodiment of FIG. 30. The embodiment of FIG. 33 also has one or more electrodes (e.g., shown as circles), which can be in the peripheral ring of the contact surface. The embodiment of FIG. 33 also has topical composition dispensing openings (e.g., shown as rectangles), which can be on the peripheral ring of the contact surface. Other arrangements are possible.

FIG. 34 shows and example embodiment of a positive pressure multi-energy therapeutic device, which can include fluid dispensing capacity and positive pressure trigger. A dispenser is attached to a container storing therapeutic agent(s).

FIG. 35 is another view of the embodiment of FIG. 34.

FIG. 36 is another view of the embodiment of FIG. 34.

FIG. 37 shows example manual tissue techniques.

FIG. 38 is a chart with information regarding example treatments, such as for headache, which treatment can target various nerves.

FIG. 39 is a chart with information regarding example treatments, such as for neck pain, which treatment can target various nerves.

### DETAILED DESCRIPTION OF CERTAIN EMBODIMENTS

Therapeutic Topical Compositions

Various therapeutic compositions are disclosed herein, as well as method of using the therapeutic compositions. The composition can include an amount of sugar or sugar alcohol, an amount of alkalizing agent, an amount of humectant or other emollient, and/or one or more additional analgesic ingredients. The composition can also include various other base ingredients, as discussed herein, such as penetration enhancers or preservatives, etc. The therapeutic composition can be applied topically. The composition can be formulated for transdermal administration, such as by administering the composition to an external surface of a body to alleviate one or more disorder symptoms, for example neurogenic pain, or to treat one or more disorders, for example neurogenic inflammation. The composition can include an amount of vehicle, which can be formulated for transdermal administration.

Embodiments of the therapeutic composition can include formulations having raw materials admixed in the exemplary weight percentages discussed herein. Numerous embodiments of the compositions can be prepared by altering the weight percentages of the raw materials within the range weight percentages described.

The composition can include an amount of sugar, which can include any carbohydrate or saccharide, including monosaccharides, disaccharides, oligosaccharides, or polysaccharides. As to particular embodiments, the sugar can include a monosaccharide, such as ribose (e.g., CAS No: 50-69-1), xylose (e.g., CAS No: 58-86-6), fructose (e.g., CAS No: 57-48-7), dextrose (glucose) (e.g., CAS No: 50-99-7), galactose (e.g., CAS No: 59-23-4), mannose (e.g., CAS No: 31103-86-3), sorbose (e.g., CAS No: 87-79-6), or the like, or combinations thereof, all of which can be obtained from Sigma-Aldrich, 3050 Spruce Street, St. Louis, Mo., USA. As to other particular embodiments, the sugar can include a disaccharide, such as sucrose (e.g., CAS No: 57-50-1), maltose (e.g., CAS No: 69-79-4), lactose (e.g., CAS No: 63-42-3), lactulose (e.g., CAS No: 4618-18-2), trehalose (e.g., CAS No: 99-20-7), cellobiose (e.g., CAS No: 528-50-7), or the like, or combinations thereof. The composition can include polysacarides.

The sugar can be generally included in an amount of about 2% to about 50% by weight of the composition. However, greater or lesser weight percentages of the sugar can be included, such as depending on the disorder symptom to be alleviated or the disorder to be treated. As to particular embodiments, the amount of sugar included in the composition can be between about 5% to about 50% by weight of the composition, between about 10% to about 40% by weight of the composition, or between about 15% to about 30% by weight of the composition. As to particular embodiments of the composition, the amount of sugar, for example dextrose, or combination of sugars included can be about 5%, about 10%, about 15%, about 20%, about 25%, about 30%, about 35%, about 40%, about 45%, or about 50% by weight of the composition, or any values or ranges therebetween, although other amounts of sugar could also be used outside of these ranges, in some implementations.

The composition can include an amount of sugar alcohol, which can include any polyol (or polyhydric alcohol) derived from a sugar. The polyol can typically include an alcohol group (CH2OH) in place of an aldehyde group (CHO) of the parent sugar. The sugar alcohol can include a polyol derived from a monosaccharide or a disaccharide, including glycerol (e.g., CAS No: 56-81-5), erythritol (e.g., CAS No: 10030-58-7), threitol (e.g., CAS No: 2418-52-2), arabitol (e.g., CAS No: 7643-75-6), xylitol (e.g., CAS No: 87-99-0), adonitol (e.g., CAS No: 488-81-3), mannitol (e.g., CAS No: 69-65-8), sorbitol (e.g., CAS No: 50-70-4), dulcitol (e.g., CAS No: 608-66-2), fucitol (e.g., CAS No: 13074-06-1), iditol (e.g., CAS No: 488-45-9), inositol (e.g., CAS No: 87-89-8), volemitol (e.g., CAS No: 30635-52-0), isomalt (e.g., CAS No: 64519-82-0), maltitol (e.g., CAS No: 585-88-6), lactitol (e.g., CAS No: 585-86-4), maltotriitol (e.g., CAS No: 32860-62-1), or the like, or combinations thereof.

The sugar alcohol can be generally included in an amount of about 2% to about 50% by weight of the composition. However, greater or lesser weight percentages of the sugar alcohol can be included, such as depending on the disorder symptom to be alleviated or the disorder to be treated. As to particular embodiments, the amount of sugar alcohol included in the composition can be between about 5% to about 50% by weight of the composition, between about 10% to about 40% by weight of the composition, or between about 15% to about 30%/o by weight of the composition. As to particular embodiments of the composition, the amount of sugar alcohol, for example mannitol, or combination of sugar alcohols included can be about 5%, about 10%, about 15%, about 200%, about 25%, about 30%, about 35%, about 40%, about 45%, or about 50% by weight of the composition, or any values or ranges therebetween, although other amounts of sugar alcohol could also be used outside of these ranges, in some implementations.

The composition can include a combination of sugar and sugar alcohol in various amounts. A combination of any number of sugar(s) and sugar alcohol(s) can be used, such as for a combined amount of about 5%, about 10%, about 15%, about 20%, about 25%, about 30%, about 35%, about 40%, about 45%, or about 50% by weight of the composition, or any values or ranges therebetween, although other amounts could also be used outside of these ranges, in some implementations. The ratio of sugar(s) to sugar alcohol(s) can be about 1 to 5, about 1 to 4, about 1 to 3, about 1 to 2, about 1 to 1.5, about 1 to 1, about 1.5 to 1, about 2 to 1, about 3 to 1, about 4 to 1, or about 5 to 1, or any values or ranges therebetween, although other proportions could also be used in some cases. By way of example, the composition can include about 10% mannitol by weight and about 10% dextrose by weight. Mannitol and dextrose can each have advantages and disadvantages, and in certain circumstances it can be advantageous to use a combination of dextrose and mannitol. For example, dextrose can be an energy source for microorganisms, such as bacteria and fungi, and mannitol can be better than dextrose for preventing microbial growth. However, mannitol can be a diuretic and can cause digestive issues (e.g., gas, bloating, and diarrhea). In sensitive individuals, even a small amount of mannitol can cause these digestive problems. Using a combination of dextrose (or another sugar) and mannitol (or another sugar alcohol) can supply a sufficiently high concentration of sugar/sugar alcohol while having a sufficiently low amount of mannitol so that the side effects of mannitol (e.g., gas, bloating and diarrhea) are minimized and also having a sufficiently low amount of dextrose so that the side effects of dextrose (e.g., microbial growth) are minimized. Mannitol is known to induce diuresis in tissue it is introduced into. At times, when addressing tissue, including superficial nerves, this may be an undesirable characteristic. In such a case, a combination of mannitol and dextrose may be used to lower overall mannitol concentration, yet preserving overall therapeutic effect.

In some embodiments, it can be advantageous to use sugar (e.g., dextrose) and not a sugar alcohol (e.g., mannitol). For example, by using dextrose and not mannitol, the diuretic and diarrhea effects of mannitol can be avoided. Also, mannitol is banned by the World Anti-Doping Agency. Sugar, such as dextrose, can be an effective alternative to mannitol.

In some embodiments, it can be advantageous to use sugar alcohol (e.g., mannitol) and not sugar (e.g., dextrose). For example, when used on tissue that is dark and/or moist or otherwise susceptible or sensitive to microbial growth, mannitol can be preferable to dextrose since mannitol is less encouraging to microbial growth than dextrose.

The composition can include an amount of alkalizing agent, which can be capable of adjusting a pH from a lesser alkalinity toward a greater alkalinity. The alkalizing agent can include L-Arginine (e.g., CAS No: 74-79-3), sodium bicarbonate (e.g., CAS No: 144-55-8), potassium citrate (e.g., CAS No: 866-84-2), calcium carbonate (e.g., CAS No: 471-34-1), calcium acetate (e.g., CAS No: 62-54-4), sodium citrate, or the like, or any of the salts discussed herein, or any combinations thereof. In some instances, L-Arginine can have anti-inflammatory and/or analgesic properties in addition to being an alkalizing agent. Also L-Arginine can be more stable than some other alkalizing agents, such as sodium bicarbonate. L-Arginine can have good skin penetration attributes. In some embodiments, the composition can include any combination of the alkalizing agents disclosed herein. By way of example, the composition can include L-Arginine (e.g., about 1% to about 5% by weight, or about 2% to about 4% by weight) and sodium bicarbonate (e.g., about 0.1% to about 1% by weight, or about 0.25% to about 0.75% by weight). One particular purpose of these pH modulators would be to deliver composition at or above normal skin pH, for example pH 4.7-5.0, pH 5.1-6.0, pH 6.1-7.0, pH 7.1-8.0, pH 8.1-9.0, pH 9.1-10 or pH 10.1-11, or any ranges between any of these values. Combinations of pH modulators allow for versatility and suitability of the therapeutic compound for different regions of the body (e.g. the face, the hands, the lower back, the armpits, the nasal passages, the mouth, the lips, the genital regions, and the limbs).

As to particular embodiments, the alkalizing agent can include a salt. As to particular embodiments, the salt can include a monovalent cation, a divalent cation, or a trivalent cation, including but not limited to sodium, calcium, potassium, zinc, iron, magnesium, or the like, or combinations thereof. As to other particular embodiments, the salt can include an anion, including but not limited to chloride, acetate, ascorbate, bicarbonate, citrate, formate, fumarate, phosphate, succinate, borate, gluconate, lactate, malate, trimalate, panthothenate, thiocyanate, glycinate, sulphate, or the like, or combinations thereof.

As to particular embodiments, the salt can be selected from the group including or consisting of: sodium chloride, sodium acetate, sodium bicarbonate, sodium citrate, sodium phosphate, sodium dihydrogen phosphate, sodium hydrogen phosphate, sodium succinate, sodium borate, sodium gluconate, sodium citrate, sodium lactate, calcium citrate, calcium chloride, calcium pantothenate, calcium gluconate, calcium phosphate, potassium chloride, monopotassium phosphate, dipotassium phosphate, tripotassium phosphate, potassium gluconate, magnesium sulphate, magnesium chloride magnesium gluconate, magnesium acetate, magnesium malate, magnesium glycinate, magnesium lactate, zinc chloride, zinc sulphate and zinc acetate. Other exemplary salts may be formed from any combination of anions and cations listed above and may include, anhydrous, hydrates, dehydrates, or the like, or combinations thereof.

The alkalizing agent(s) can be generally included in an amount of about 0.1% to about 20% by weight of the composition. However, greater or lesser weight percentages of the alkalizing agent can be included, such as depending on the disorder symptom to be alleviated or the disorder to be treated. As to particular embodiments, the amount of alkalizing agent included in the composition can be between about 1% to about 20% by weight of the composition, between about 1.5% to about 15% by weight of the composition, or between about 2% to about 10% by weight of the composition. As to particular embodiments of the composition, the amount of alkalizing agent, such as L-Arginine, or combination of alkalizing agents included can be about 0.1%, about 0.3%, about 0.5%, about 0.7%, about 1%, about 1.5%, about 2%, about 3%, about 5%, about 10%, about 15%, about 20% by weight of the composition, or any values or ranges therebetween, although other amounts of alkalizing agent(s) could also be used outside of these ranges, in some implementations.

As to particular embodiments, the amount of alkalizing agent(s) included in the composition can be sufficient to provide the composition with a pH of between about 8 to about 10. As to particular embodiments, the amount of alkalizing agent included in the composition can be sufficient to provide the composition with a pH of about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.75, about 8.0, about 8.25, about 8.5, about 8.75, about 9, about 9.25, about 9.5, about 9.75, or about 10, or any values therebetween, or any ranges bounded by any combination of these values, although other amounts could also be used.

As to particular embodiments, the amount of alkalizing agent(s) included in the composition can be sufficient to elevate the pH of body tissue. The human body can ordinarily have a pH of between 7.35 and 7.45 and the skin between 4.6 and 4.8. The composition can have sufficient alkalizing agent(s) so that when the composition is applied (e.g., transdermally), the alkalinity of the underlying tissue (e.g., perineural environment proximate a nerve) can be raised, such as above 7.45 or about 7.5 while the skin surface can be raised above 4.7. In some embodiments, it may be sufficient to simply raise the skin surface pH above 4.6 to exert an effect, while lowering the risk of skin irritation in particularly sensitive individuals. As to particular embodiments, upon transdermal administration, the composition can be effective to alkalize underlying tissue (e.g., a perineural environment proximate a nerve) to have a pH of about 7.5, about 7.75, about, about 8.25, about 8.5, about 8.75, about 9, about 9.25, about 9.5, about 9.75, or about 10, or any values therebetween, or any ranges bounded by any combination of these values, although other values are possible in some implementations. As to particular embodiments, upon transdermal administration, the composition can be effective to alkalize underlying tissue (e.g., a perineural environment proximate a nerve) to have a pH of between about 8 and about 9. In some instance, a composition that is effective to alkalize the underlying tissue to have a pH of between about 7.75 and about 8 can be advantageous, such as for patients with sensitive skin. For patients with particularly sensitive skin, it may be beneficial to use a gentler alkalizing effect to move the skin surface pH to about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.75, about 8.0, about 8.25, about 8.5, about 8.75, about 9, about 9.25, about 9.5, about 9.75, or about 10, or any values there between, or any ranges bounded by any combination of these values. The latter effect may be more appropriate for an embodiment more suited for cosmetic or longer term use.

The amount of alkalizing agent(s) can depend on the other ingredients of the composition. For example, different amounts of alkalizing agent(s) may be used depending on whether the rest of the ingredients are acidic, neutral, or alkaline. Also, different compositions can tolerate different amounts of the alkalizing agent(s). The pH can affect the activity and the stability of the composition. A pH that can yield high efficacy for bioactivity might be unstable, whereas a composition with a pH that results in high stability might not have sufficient bioactivity. Accordingly, an optimal pH range can be different for different compositions. An optimal pH has been obtained through several embodiments of the described therapeutic compound at a % of total volume of around 2-10% (e.g., with a mean of 7%). In another embodiment optimal pH has been achieved with combination of 0.93% sodium bicarbonate, 2.24% L-Arginine, and 2.24% caprylic acid, by volume.

The composition can include a vehicle, which can include one or more excipients in which the sugar or sugar alcohol, and/or other ingredients, can be solubilized or suspended. As to particular embodiments, the excipient can render the composition suitable for topical administration or transdermal administration, whereby the vehicle can facilitate transdermal administration of a portion of the amount of sugar or sugar alcohol and/or other ingredients. As illustrative examples, the composition including the amount of sugar or sugar alcohol, the amount of alkalizing agent(s), and/or other ingredients, and the amount of vehicle can take the form of lotion, cream, emulsion, ointment, gel, foam, paste, oil, lipid delivery system, spray, drops, or the like, or combinations thereof.

As to particular embodiments, the composition (e.g., the vehicle) can include an emulsion base, which can have an oil phase. As illustrative examples, the oil phase can include vegetable oils, animal oils, mineral oils, silicone oils, synthetic oils, fatty acids, fatty alcohols, phospholipids, paraffin waxes, or the like, or combinations thereof.

As to particular embodiments, the composition (e.g., the vehicle) can include one or more solubilizing agents, such as cyclodextrins, surfactants, organic solvents, alcohols, polysorbates, or the like, or combinations thereof.

As to particular embodiments, the composition (e.g., the vehicle) can include one or more viscosity-increasing agents, such as microcrystalline cellulose, carboxymethylcellulose sodium, propylene glycol alginate, xanthan gum, polyacrylic acid, or the like, or combinations thereof.

As to particular embodiments, the composition (e.g., the vehicle) can include one or more emulsifying or co-emulsifying agents, such as non-ionic surfactants, polyethylene glycol esters, polyoxypropylene glycol ethers, sorbitan esters, ethoxylated sorbitan esters, poly esters, or the like, or combinations thereof.

As to particular embodiments, the composition (e.g., the vehicle) can include one or more emulsion stabilizing agents, such as abietic acid, hydrogenated lanolin alcohol, calcium myristate, hydroxyaluminium distearate, aluminum isostearate, aluminum stearate, 7-dehydrocholesterol, 8-dehydrocholesterol, aluminum magnesium hydroxide, stearic acid, lauryl alcohol, hydroxyethyl cellulose, or the like, or combinations thereof.

As to particular embodiments, the composition (e.g., the vehicle) can include one or more preservatives or preserving agents, such as sorbic acid, methyl paraben, propyl paraben, benzoic acid, sodium benzoate cetrimide, phenoxyethanol, chlorphenisin, methylchloroisothiazolinone, or the like, or combinations thereof.

As to particular embodiments, the composition (e.g., the vehicle) can include one or more penetration enhancers, such as alcohols, sulphoxides, azone, pyrrolidones, urea, disubstituted aminoacetates, glycols (for example, propylene glycol), surfactants, terpenes, terpenoids, fatty acids, esters, cyclodextrins, phospholipids, or the like, or combinations thereof.

As to particular embodiments, the composition (e.g., the vehicle) can include water (e.g., CAS No: 7732-18-5), which can be filtered, de-ionized, distilled, or water otherwise filtered or purified.

Where trade names or trademarks are utilized herein, the trade name material or the trademark material is understood to have the chemicals or ingredients in the amounts or combinations as described or as known. The trade name material or trademark material or a substantially equivalent product or combination of chemicals or ingredients can be utilized in embodiments of the composition. It is further understood that where a trade name material or trademark material is utilized that substantially equivalent chemicals or ingredients in the amounts and combinations as indicated can be utilized in substitution of the trade name material or trademark material. A person of ordinary skill in the art can convert the weight or volume percentages disclosed to determine the amount of each chemical or ingredient to mix when the equivalent of the trade name material or trademark material is prepared.

As to particular embodiments, the composition (e.g., the vehicle) can include an amount of magnesium. As to particular embodiments, the amount of magnesium can be provided by magnesium chloride, magnesium sulfate, or the like, or combinations thereof.

As to particular embodiments, the composition (e.g., the vehicle) can include an amount of Aesculus hippocastanum. As to particular embodiments, the amount of Aesculus hippocastanum can be in a range of between about 0.25% to about 2% by weight of the composition. As to particular embodiments, the amount of Aesculus hippocastanum can include an amount of aescin.

As to particular embodiments, the composition (e.g., the vehicle) can include an amount of quercetin. As to particular embodiments, the amount of quercetin can be in a range of between about 0.1% to about 1% by weight of the composition.

As to particular embodiments, the composition (e.g., the vehicle) can include an amount of acetyl-L-carnitine. As to particular embodiments, the amount of acetyl-L-carnitine can be in a range of between about 0.025% to about 3% by weight of the composition.

As to particular embodiments, the composition (e.g., the vehicle) can further include an amount of zinc. As to particular embodiments, the amount of zinc can be in a range of between about 0.025% to about 3% by weight of the composition. As to particular embodiments, the amount of zinc can be provided by zinc oxide, zinc sulfate, or the like, or combinations thereof.

As to particular embodiments, the composition (e.g., the vehicle) can include one or more colorants, fragrances, or the like, as persons of ordinary skill in the art would understand.

As to particular embodiments, upon transdermal administration, the composition can be effective to decrease neurogenic pain, decrease neurogenic inflammation, or combinations thereof.

As to particular embodiments, upon transdermal administration, the composition can be effective to alkalize a perineural environment proximate a nerve. As to particular embodiments, alkalization of the perineural environment can include adjusting a pH of the perineural environment from a lesser alkalinity toward a greater alkalinity.

As to particular embodiments, upon transdermal administration, the composition can be effective to decrease an amount of cations in a perineural environment proximate a nerve.

As to particular embodiments, upon transdermal administration, the composition can be effective to decrease an amount of hydrogen ions (H+) in a perineural environment proximate a nerve. Without being limited by theory, it is believed that there is an association between tissue acidity and neurogenic inflammation whereby hydrogen ions (H+) may prime the transient receptor potential cation channel subfamily V member (TrpV1), thereby increasing neurogenic inflammation and associated neurogenic pain. Accordingly, decreasing the amount of hydrogen ions (H+) by adjusting the perineural environment from a lesser alkalinity toward a greater alkalinity may be beneficial for alleviating neurogenic pain or treating neurogenic inflammation.

As to particular embodiments, upon transdermal administration, the composition can be effective to decrease a viscosity of hyaluronic acid (also known as hyaluronan, hyaluronate, or HA), which is an anionic, nonsulfated glycosaminoglycan widely distributed throughout connective, epithelial, and neural tissues. Particularly, hyaluronic acid can be found between fascial layers, acting as a lubricant to facilitate fascial glide. As peripheral nerves, especially superficial sensory nerves, can typically be enveloped between fascial layers, decreasing the viscosity of hyaluronic acid may decrease friction or mechanical irritation of the nerves by the fascia enveloping the nerve.

Adjusting the pH of a perineural environment from a lesser alkalinity toward a greater alkalinity may result in a conformational change in the hyaluronic acid molecule resulting from a degradation of attraction forces between hyaluronic acid molecules, thereby increasing the flexibility of the hyaluronic acid polymer and correspondingly decreasing the viscosity of hyaluronic acid.

As to particular embodiments, upon transdermal administration, the composition can be effective to provide an amount of energetic substrate to a nerve, whereby as to particular embodiments, the energetic substrate can be a sugar, for example dextrose. Normal mitochondrial function in Schwann cells can be beneficial for maintaining axon-glial interactions which can support long-term support of axons and normal peripheral nerve function. As peripheral neurogenic inflammation may contribute to compartmental edema and consequently, to local sequestration of superficial sensory nerves which may lead to energy substrate deprivation, for example by impaired axoplasmic interstitial flow and impaired perineural interstitial flow, the delivery of an amount of energetic substrate to the nerve may promote nerve function, thereby decreasing neurogenic pain, decreasing neurogenic inflammation, or combinations thereof.

Transdermal drug delivery can be beneficial over oral drug delivery. Transdermal drug delivery can transport a drug or macromolecules painlessly through skin into the blood circulation (e.g., at a relatively fixed rate). Topical administration can avoid hepatic first pass metabolism, and can enhance therapeutic efficiency and maintenance of steady plasma level of the drug. Human skin surface, as a site of drug application for both local and systemic effects, is the most eligible candidate available. Transdermal administration can be used, intranasal administration can be used, and transmucosal adsorption can be used. Intranasal administration and transmucosal adsorption can be particularly useful for treatment of headache disorders. Also, the therapeutic compositions disclosed herein can be administered as eye drops, such as for treating eye irritation, inflammation, and/or pain.

Therapeutic Compositions Containing Humectant

Skin hydration can increase permeability of therapeutic agents, in some cases. In some embodiments, the skin (or other application area) can be pre-soaked, such as with water or other preparatory fluid. A skin hydrating agent can be used, as discussed herein. In some cases, water can be used to help with friction or other administration techniques (e.g., scraping, microneedling, microabrasion, and Ultrasound). Also, in some instances, dilution of the therapeutic agents can be beneficial, as discussed herein. Because of the osmotic effect, hyperosmalar agents can provoke receptors (e.g., TRP receptors).

The composition can include a skin-hydrating agent, which can be a humectant. As to particular embodiments, the composition can include an amount of one more humectants. The humectant can include glycerine or glycol (e.g., CAS No: 56-81-5), caprylic/capric triglyceride (e.g., CAS No: 73398-61-5), sodium PCA (e.g., CAS No: 28874-51-3), xanthan gum (e.g., CAS No: 11138-66-2), sodium hyaluronate (e.g., CAS No: 9067-32-7), ceresin wax (e.g., CAS No: 8001-75-0), hyaluronic acid (e.g., CAS No: 9004-61-9), c30-45 alkyl methicone (e.g., CAS No: 246864-88-0), c30-45 alkyl olefin (e.g., CAS No: 222400-22-8), microcrystaline wax (e.g., CAS No: 63231-60-7), polyethylene (synthetic wax) (e.g., CAS No: 9002-88-4), and/or silica dimethyl silylate, or any combinations thereof. A particularly beneficial combination of humectants for a therapeutic composition includes Caprylic Capric Triglyceride, Cerecin wax, sodium hyalonurate, and Glycerin Stearate. Skin hydrating agents (e.g., humectants) that can be used for the composition include sugars (e.g., glucose, fructose, sucrose, honey, and their derivatives), proteins, amino acids, elastin, and collagen, silicone waxes, sodium PCA (sodium L-pyroglutamate (e.g., about 0.5% to 10%), butylene glycol (which can have humectant, solvent, and/or antimicrobial properties), glycoproteins, collagen protein, Hexanediol CG, elastic protein, ethoxydiglycol, honey extract, jojoba, ceramide, sucrose cocoate, Glycerin (Glycerol), Sorbitol, PEG (polymerization of ethylene glycol), Propylene Glycol, Aloe Vera, Urea, Algae Extract, Sodium Hyaluronate (polysacharride), Lanolin, moisturizes, and hydrates, or any combinations thereof. Humectants can draw water to the skin from the humidity in the environment and/or can enhance water absorption from the skin.

As to particular embodiments, the amount of humectant (or combination of humectants) can be in a range of between about 2% and about 70%, or between about 10% and about 25%, of total volume of the composition. Multiple humectants can be combined, such as with each humectant having a range of between about 0.5% and about 15% of total volume for the composition.

When the composition is applied topically, the therapeutic ingredients can penetrate through the skin and into the underlying tissue, such as to the nerves. The humectant can improve penetration, and can help produce an appropriate concentration of the therapeutic ingredient(s) in the underlying tissue for an extended period of time. The humectant can operate as an extended release mechanism. Humectants are different from emollients, and a humectant can pull moisture from the surrounding environment. Humectants can increase the water content of the skin itself, and can increase water absorption into the skin. More humidity in the skin can help improve skin permeability and penetration of the therapeutic composition. Without being limited by theory, it is believed that increasing the water content of the skin cells can cause the skin cells to become engorged, which can produce spaces between the skin cells that the therapeutic composition can pass through.

The humectant can draw water to the surface of the skin (e.g., from the environment), and as that water is absorbed into the skin, the water can pull the therapeutic ingredients (e.g., sugar, sugar alcohol, alkalizing agent, or others disclosed herein) into the tissue as well. By extending the time during which the skin absorbs moisture, the humectant can also increase the amount of time during which the therapeutic components are absorbed into the tissue as well. The humectant can increase the total amount of the composition that gets absorbed into the tissue. With reference to a composition that includes sugar, by way of example, the humectant can increase the amount of sugar that gets actually delivered to the underlying tissue. In some cases, especially for compositions that do not use a humectant, the sugar in the composition that is not absorbed is left on the skin as a tacky residue, which can be unpleasant, especially for patients or locations with hairy skin. Also, the residue portion of the composition is wasted since it is not absorbed. The humectant can increase absorption of the composition, which can reduce or eliminate the tacky residue, increase effectiveness, and in some cases can reduce the amount of the composition needed for treatment. The humectant can also increase the length of time over which the composition is absorbed into the skin, which can improve effectiveness and can reduce the frequency of reapplying the composition.

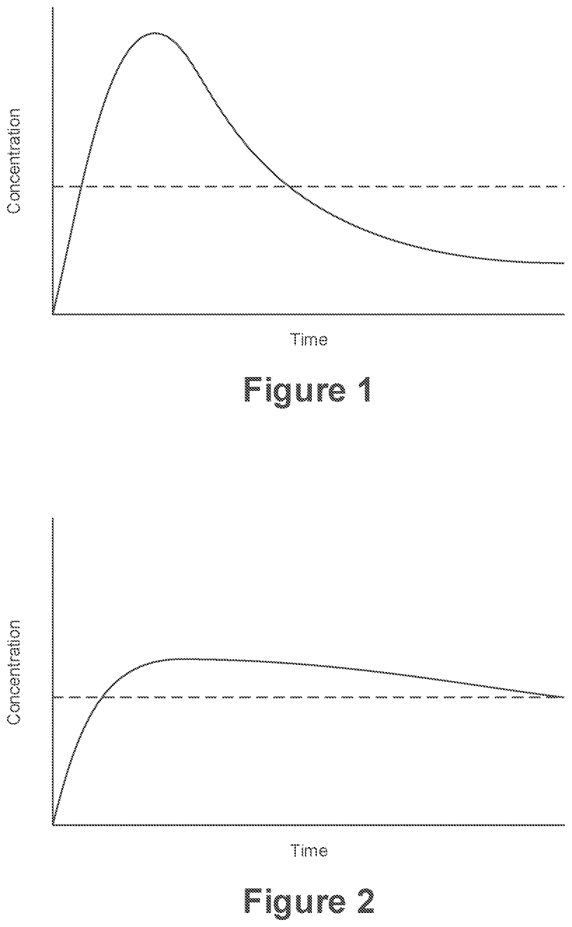

The humectant can also facilitate regulation of the concentration of the therapeutic ingredients in the underlying tissue. In order to produce a desired concentration at the nerves, a higher concentration of the therapeutic composition is applied topically to the surface of the skin. With reference to sugar, for example, in some cases a 5% concentration of sugar at the nerve can be therapeutic for a particular treatment. However, in order to produce the 5% concentration of sugar at the underlying nerve for a suitable amount of time, a higher concentration of sugar (e.g., 20%) can be applied to the surface of the skin. In some cases, the relatively high concentration applied to the skin can cause an initial spike in the concentration in the underlying tissue followed by the concentration tapering off. In some cases, in order to have the concentration above the target amount (e.g., 5%) for a sufficient time, the initial spike can be well above the target concentration (e.g., 5%), as shown conceptually in FIG. 1. Including a humectant in the composition can reduce the initial spike in the concentration by effectively diluting the composition that reaches the underlying tissue, and the humectant can also extend the period of time over which the composition is drawn into the tissue, thereby keeping the concentration above the target amount (e.g., 5%) for a longer period of time, as shown conceptually in FIG. 2. With the humectant, the composition can be configured to penetrate into the underlying tissue little by little with the water. The humectant can provide an extended release function to the composition. The humectant can increase the amount of moisture in the underlying tissue as well, which can decrease dehydration of the underlying tissue (e.g., around nerve receptors and/or pathways).

In some implementations, the composition can be applied with a bandage or tape or other covering, such as according to the embodiments disclosed in U.S. Patent Application Publication No. 2017/0360867. The covering can help hold in hydration, which can increase permeability of the skin. The covering can increase penetration of the composition into the underlying tissue, and can increase the amount of the composition that gets absorbed (e.g., reducing the tacky residue as discussed), and can extend the period of time over which the composition is absorbed, which can reduce the frequency of reapplication. When used together with a composition that includes a humectant, the bandage, patch, or other covering can be particularly effective. For example, a single application of the composition with a humectant and using a bandage could be used in some instances to provide similar treatment to multiple topical applications throughout the day of a composition without the humectant or bandage.

Particular Ingredients for Therapeutic Compositions

Various possible additional ingredients for the therapeutic compositions are discussed herein, and can be used in any suitable combination together with the sugar, sugar-alcohol, alkalizing agent, vehicle, or any combinations thereof.

As to particular embodiments, the composition can include an amount of pterostilbene (e.g., CAS No: 537-42-8). As to particular embodiments, the amount of pterostilbene can be in a range of between about 0.25% to about 40%, or about 0.25% to about 20% by total volume or weight of the composition, or a concentration of about 5-100 mg/ml. Pterostilbene can be beneficial for treatment of neuropathy. In one embodiment Pterostilbene can be included in the therapeutic compound in the form of one or more fruit extracts. Blueberry (Vaccinium corymbosum) and/or Bilberry Fruit Extract (e.g., CAS No: 84082-34-8; 1160262-40-7) (example range of about 0.5-5% by total volume or weight of the composition), Acai Fruit Extract (e.g., CAS No: 879496-95-4), Grape Seed Extract (e.g., CAS No: 84929-27-1), Vitis vinifera (Grape Seed) Oil (e.g., CAS No: 84929-27-1/8024-22-4) and Plum Extract (e.g., CAS No: 56-81-5, 7732-18-5, 1176234-54-0, 122-99-6) or Kukudu plum oil (CAS: 1542150-12-8) (e.g., about 0.5-5% of total volume or weight) are beneficial for this purpose in that it has been found that in solution these Pterostilbene-containing compounds are stable and effective. In one embodiment, the amount of grape seed extract (or any combination of the aforementioned fruit extracts) can range between about 20-2,500 mg/ml (or about 0.25-40% of total volume or weight). Without being bound by theory, it is believed that pterostilbene, like phytosterols and TOR modulators, support mitochondrial and energetic functions of the cell, supporting regeneration of damaged cells. In some embodiments, the composition can include one or more phytosterols or TOR modulators.

As to particular embodiments, the composition can include an amount of vitamin D3. As to particular embodiments, the amount of vitamin D3 can be in a range of between about 0.5% to about 10%, or between about 0.2% and about 20%, or about 5%, by total weight or volume of the composition. Vitamin D3 can be beneficial for treatment of neuropathy. Some studies have provided evidence that vitamin D deficiency may be associated with pain sensitivity in diabetic polyneuropathy.

As to particular embodiments, the composition (e.g., the base or vehicle) can include an amount of one or more of the following, at least some of which can be penetration enhancers or can improve absorption: an oil, a vegetable oil, argan oil, jojoba oil, shea butter, cocoa butter, grape seed oil, coconut oil, isopropyl palmitate (e.g., CAS No: 142-91-6), sodium phytate (e.g., CAS No: 14306-25-3), octyl palmitate (e.g., CAS NO: 16958-85-3) (also called ethylhexyl palmitate), d-Limonene (e.g., CAS No: 5989-27-5), Tetrasodium EDTA (e.g., CAS No: 64-02-8), aloe (e.g., cold-pressed aloe), or arnica, or any combinations thereof. As to particular embodiments, the amount of each of these components can be in a range of between about 1% to about 25% by weight of the composition. D-limonene was found to enhance analgesic effect and was useful in neutralizing smell and sensation of otherwise potentially noxious ingredients, such as menthol. The vegetable oils listed herein were found to result in a lower comedogenic effect as compared to other emollients and penetration enhancers.

As to particular embodiments, the composition can include an amount of one or more preservatives or antimicrobial components, such as naticide (which is a vegetable-based fragrance with a vanilla scent that is effective against gram+ bacteria, gram− bacteria, yeasts, and molds, and can be obtained from Sinerga, Via della Pacciarna 67, 21050 Gorla Maggiore, Italy), radish root, phenoserve (e.g., Phenoxyethanol, Methylparaben, Ethylparaben, Butylparaben and Propylparaben), phenoip (e.g., Phenoxyethanol, Methylparaben, Ethylparaben, Butylparaben, Propylparaben and Tsobutylparaben), or optiphen (e.g., phenoxyethanol and capylyl glycol), or any combinations thereof. As to particular embodiments, the amount of each of these components can be in a range of between about 0.5% to about 10% by weight of the composition.

Although any suitable combination of the ingredients, as disclosed herein, can be used, some ingredients can provide advantages over other ingredients, in some cases depending on the particular application or treatment. For example, in some instances, the composition can be configured to have low comedogenicity so that it can avoid or impede clogging pores or causing pimples. This can be particularly advantageous when the composition is used on the face, such as for treatment of headache or facial pain. In some embodiments, all ingredients of the composition can have a comedogenicity rating of 3/5 or less, or 2/5 or less, or 1/5 or less, or 0/5. In some embodiments, the ingredients of the composition that make up about 95%, about 90%, about 85%, about 80%, about 75%, about 70%, about 65%, about 60%, about 55%, or about 50% by weight of the composition (or any values or ranges therebetween) can have a comedogenicity rating of 3/5 or less, or 2/5 or less, or 1/5 or less, or 0/5. In some embodiments, all ingredients of the base or vehicle of the composition can have a comedogenicity rating of 3/5 or less, or 2/5 or less, or 1/5 or less, or 0/5. In some embodiments, the ingredients of the composition that make up about 95%, about 90%, about 85%, about 80%, about 75%, about 70%, about 65%, about 60%, about 55%, or about 50% by weight of the base or vehicle of the composition (or any values or ranges therebetween) can have a comedogenicity rating of 3/5 or less, or 2/5 or less, or 1/5 or less, or 0/5. In some embodiments, all inactive ingredients of the composition can have a comedogenicity rating of 3/5 or less, or 2/5 or less, or 1/5 or less, or 0/5. In some embodiments, about 95%, about 90%/6, about 85%, about 80%, about 75%, about 70%, about 65%, about 60%, about 55%, or about 50% by weight of the inactive ingredients of the composition (or any values or ranges therebetween) can have a comedogenicity rating of 3/5 or less, or 2/5 or less, or 1/5 or less, or 0/5. Examples are provided herein, which have been formulated with combinations of non-comedogenic ingredients, while also enhancing analgesic and anti-inflammatory effect of the total composition.

Comedogenicity was originally determined in the 1970's by Klingman and Fulton, who pioneered the rabbit ear test. This involved applying a substance to the inner ear of a rabbit, and observing for the development of clogged pores. This serious of test contributed to current understanding of comedogenicity as well as current comedogenic ratings. Subsequent investigators have contributed to understanding of comedogenicity of ingredients including Fulton (1989) and Morris & Kwan (1983). Tests utilized include back skin test, use of subjects with larger pores and use of occlusive bandage (References: J E Fulton, Comedogenicity and irritancy of commonly used ingredients in skin care products, J Soc Cosmet Chem 1989, 40, 321-333; WE Morris and SC Kwan, Use of the rabbit ear model in evaluating the comedogenic potential of cosmetic ingredients, J Soc Cosmet Chem 1983, 34, 215-225.)

By way of example, some penetration enhancers can be comedogenic, and can clog pores and cause pimples and other skin ailments. For example, isopropyl palmitate can be used as a penetration enhancer and/or an emollient, but has high comedogenicity (e.g., a rating of 4/5). Isopropyl palmitate can cause pustular acne or other skin ailments. Other substances that include isopropyl can also be comedogenic (e.g., isopropyl myristate with comedogenicity of 5/5). Other substances with lower comedogenicity can be used in place of isopropyl palmitate (or in place of other isopropyl-containing substances). By way of example, the composition (e.g., the base or vehicle) can include shea butter (e.g., 0/5 to 2/5 comedogenicity), sunflower oil (e.g., 0/5 to 2/5 comedogenicity), natural cold-pressed plant oils, castor oil (1/5 comedogenicity), squalene (e.g., CAS No: 111-01-3) (e.g., 1/5 comedogenicity), jojoba oil (2/5 comedogenicity), argan oil (0/5 comedogenicity), jojoba oil (e.g., 0/5 to 2/5 comedogenicity), hemp seed oil (0/5 comedogenicity).

In some embodiments, the composition can include cocoa butter, grape seed oil, coconut oil, sodium phytate, octyl palmitate (e.g., CAS NO: 16958-85-3), or ethylhexyl palmitate.

As to particular embodiments, the composition (e.g., the base or vehicle) can include argan oil, jojoba oil, shea butter, or a combination thereof, and in some cases can be supplemented with a terpene (e.g., d-limonene). The composition (e.g., the base or vehicle) can also include Tetrasodium EDTA, which can improve penetration.

In some embodiments, the composition (e.g., the base or vehicle) can include caprylic/capric triglyceride (e.g., comedogenicity of 0/5), which can be used as an emollient. In some embodiments, the composition (e.g., the base or vehicle can have a combination of c30-45 alkyl methicone, c30-45 alkyl olefin, microcrystaline wax, polyethylene (e.g., synthetic wax), and silica dimethyl silylate (e.g., which combination can have 0/5 comedogenicity), and which can be an emollient.

In some embodiments, the composition does not include isopropyl palmitate (e.g., 4/5 comedogenicity). In some embodiments, the composition does not include isopropyl myristate (e.g., 5/5 comedogenicity). In some embodiments, the composition does not include any substance that includes isopropyl. In some embodiments, the composition does not include octyl palmitate (e.g., 4/5 comedogenicity).

Ceteareth-20 (e.g., CAS No: 68439-49-6) can be used as an emulsifier. However, ceteareth-20 can be comedogenic (e.g., 4/5 comedogenicity). Ceteareth-20 can also be an allergen and can cause skin irritation and may introduce carcinogens. Accordingly, while some embodiments of the composition can include ceteareth-20, in other embodiments the composition does not include ceteareth-20. The composition (e.g., the base or vehicle) can include other emulsifiers, such as that are less comedogenic, such as xanthan gum (0/5 comedogenicity), tapioca starch (0/5 comedogenicity), or carnuba wax (1/5 comedogenicity).

In some cases, the composition can include cetearyl alcohol (e.g., CAS No: 8005-44-5), which can be used as an emulsifier (e.g., emulsion stabilizer) and/or as an emollient. However, cetearyl alcohol can be comedogenic (e.g., 4/5 comedogenicity). Accordingly, in some embodiments, the composition does not include cetearyl alcohol. Other emollients or emulsifiers can be used, for example which can be less comedogenic, such as ceresin was (0/5 comedogenicity) or other emollients discussed herein.

In some embodiments, the composition can include glyceryl stearate (e.g., CAS No: 31566-31-1), which can be used as an emulsifier. However, glyceryl stearate can be comedogenic (e.g., 3/5 comedogenicity). Accordingly, in some embodiments, the composition does not include glyceryl stearate. Other emulsifiers can be used, for example which can be less comedogenic, such as lanolin wax (1/5 comedogenicity) or other emulsifiers disclosed herein.

In some embodiments, the composition can include polyethylene glycol 100 stearate (PEG 100 stearate) (e.g., CAS No: 9004-99-3), which can be an emulsifier, emollient, or surfactant. However, PEG 100 stearate can be comedogenic (e.g., 3/5 comedogenicity). Also, PEG 100 stearate can have issues with toxicity and irritation, especially on broken or irritated skin. Accordingly, in some embodiments, the composition does not include PEG 100 stearate. Other substances can be used, which can be less comedogenic, such as the various emollients, emulsifiers, and/or surfactants described herein. In some cases, the composition (e.g., the base or vehicle) can include behentrimonium methosulfate (e.g., CAS No. 81646-13-1), or behentrimonium chloride (e.g., CAS No. 17301-53-0), or a combination thereof. The composition (e.g., the vehicle or base) can include shea butter, cocoa butter, or glycerin, or any combination thereof, for example as an emollient, and various other emollients disclosed herein can also be used. In some cases, the composition (e.g., the vehicle or base) can include glycol stearate (e.g., CAS No: 111-60-4), for example as an emulsifier, and various other emulsifiers disclosed herein can also be used. In some cases, the composition (e.g., the vehicle or base) can include cocoamidopropyl betaine (e.g., CAS No: 61789-40-0), for example as an surfactant, and various other surfactants disclosed herein can also be used.

In some embodiments, the composition (e.g., the vehicle or base) can include dimethicone (e.g., CAS No: 9006-65-9), which can be used as an emollient and/or as an anti-foaming agent. In some embodiments, the composition (e.g., the vehicle or base) can include cyclomethicone (e.g., CAS No: 69430-24-6), which can be used as an emollient and/or as an anti-foaming agent. Cyclomethicone (e.g., 0/5 comedogenicity) can be less comedogenic than dimethicone (e.g., 1/5 comedogenicity). In some cases, the composition does not include dimethicone. In some cases, the composition can use both cyclomethicone and dimethicone.

In some embodiments, the composition (e.g., the vehicle or base) can include octyldodecanol (e.g., CAS No: 5333-42-6), which can be used as an emollient, emulsifier, and/or a solvent. Octyldodecanol is a long-chain fatty alcohol. Octyldodecanol can impede a formulation from separating into its oil and liquid components. Octyldodecanol can also be used as an anti-foaming agent in the composition.

In some embodiments, the composition (e.g., the vehicle or base) can include lecithin (e.g., CAS No: 8002-43-5), which can be used as an emulsifier and/or as an organogelator, which can aid with delivery. In some embodiments, the composition can include lecithin organogel, PLO organogel, pluronic lecithin organogel, MBG organogel, sorbitan organogel, polyethylene organogel, or any combinations thereof. The composition can omit lecithin and/or any of the oranogels disclosed herein.

Although some embodiments can include propylene glycol, such as for a penetration enhancer. Propylene glycol can be a PEG compound. In some cases, propylene glycol can cause skin irritation and/or tissue damage. Accordingly some embodiments can use other penetration enhancers and not propylene glycol. For example, the composition (e.g., the vehicle or base) can include terpenes, glycerol (e.g., vegetable glycerine), or propanediol (e.g., CAS No: 504-63-2), or any combinations thereof. In some embodiments, the composition does not include propylene glycol. Terpenes can be naturally occurring volatile oils and can include hydrocarbons and their oxygenated derivatives. Terpenes can be used in the composition as penetration enhancers. The composition can include one or more terpenes extracted or derived from a Cannabis plant.

In some embodiments, the composition (e.g., the base or vehicle) can include menthol (e.g., CAS No: 2216-51-5 and/or 89-78-1), which can be used as a penetration enhancer. In some cases, menthol can be used as a therapeutic component of the composition, for example as an analgesic. Menthol can also activate the TRP receptor family. Menthol can have a strong smell. Accordingly, in some embodiments, the composition does not include menthol. Other terpenes can be used, for example, as discussed herein. In some cases, the composition can include limonene (e.g., d-limonene (e.g., CAS No: 5989-27-5)), linalool (e.g., CAS No. 78-70-6), farnesol (e.g., CAS No: 4602-84-0), or cineole (e.g., CAS No: 470-82-6), or any combinations thereof. In some embodiments, camphor (CAS No: 76-22-2) (e.g., 2/5 comedogenicity) can be used in lieu of menthol or in combination with menthol. For example, the composition can have about 0.1% to about 3% by weight of camphor. By way of example, the composition can include a combination of camphor and d-limonene, and in some cases does not have menthol.

In some embodiments, the composition can include an amount of sugar (e.g., dextrose) and an amount of menthol (and in some cases additional ingredients as discussed herein). The combination of sugar (e.g., dextrose) and menthol can be advantageous over a composition with mannitol and menthol because mannitol is a diuretic and can cause diarrhea in some cases. Using a penetration enhancer, such as menthol, with mannitol can increase the amount of mannitol that gets into the circulator system and/or into the gastrointestinal tract, which can amplify side effects of mannitol. By using a sugar (e.g., dextrose) rather than mannitol in the combination with menthol, these side effects and be reduced or avoided.

In some embodiments, the composition (e.g., the base or vehicle) can include ethyhexylglycerin (e.g., CAS No: 70445-33-9), which can serve as a surfactant, a preservative, preservative enhancer, deodorizer, and/or skin conditioner. Ethyhexylglycerin can irritate skin in some instances. In some cases, the composition does not include ethyhexylglycerin. In some cases, the composition (e.g., the base or vehicle) can include one or more parabens, which can be used as a preservative, although in some embodiments, parabens can be omitted.

In some embodiments, the composition (e.g., the base or vehicle) can include phenoxyethanol (CAS No: 122-99-6), which can be a preservative. For example, phenoxyethanol, 2-phenoxyethanol, or Euxyl K 400 (e.g., a mixture of phenoxyethanol and 1,2-dibromo-2,4-dicyanobutane) can be used. Phenoxyethanol can cause allergic reactions in some cases, such as hives, eczema, and anaphylaxis. In some cases, the composition does not include phenoxyethanol. Naticide, radish root, Tetrasodium EDTA, Tetrahydrate, Methyl Paraben, Optiphen, Phenoxyethanol, Potassium Sorbate, Propyl Paraben, Tetrasodium Glutamate Diacetate, phenoserve, or any combinations thereof can be used (e.g., as a preservative).

In some embodiments, the composition (e.g., the base or vehicle) can include an amount of polysorbate 20, e.g., which can be an emulsifier. In some cases, borax (e.g., sodium borate) mixed with beeswax can be used (e.g., as an emulsifier and/or alternative to polysorbate 20.

In some embodiments, the composition (e.g., the base or vehicle) can include an amount of isopropyl myristate, e.g., which can be used as an emollient and/or penetration enhancer. Isopropyl myristate can be comedogenic (e.g., 5/5 comedogenicity), and can be omitted in some implementations. Beeswax, olive oil, lanolin, or any combinations thereof can be used for emollients, as well as the other emollient ingredients discussed herein. Tetrasodium EDTA can be used for improving skin penetration. In some embodiments, highly unsaturated oils (e.g., containing linoleic fatty acid, linolenic fatty acid, omega 3 fatty acid, and/or omega 6 fatty acid) can be used for the composition. Alternatively, those ingredients can be omitted from the composition, for example, since those ingredients can be susceptible to oxidation and can be difficult to stabilize. Also, peroxides formed from these ingredients can be reactive with other ingredients of the composition, and can cause irritation.

In some embodiments, the composition (e.g., the base or vehicle) can include an amount of stearic acid, although it can be omitted in some embodiments. In some embodiments, the composition (e.g., the base or vehicle) can include an amount of Disodium Benzophenone-4 (e.g., CAS No: 4065-45-6), although it can be omitted in some embodiments (Benzophenone-4 can be a skin toxicant or allergen in some cases). In some embodiments, the composition (e.g., the base or vehicle) can include an amount of Iodopropynyl Butylcarbamate (IPBC) (e.g., CAS No: 55406-53-6), which can be a fungicide. In some cases, Iodopropynyl Butylcarbamate can be an allergen, and can be omitted in some implementations. In some embodiments, the composition (e.g., the base or vehicle) can include an amount of vitamin E, although it can be omitted in some embodiments. Various types of gelators can be used for the composition (e.g., the base or vehicle), such as sterol, sorbitan monostearate, lecithin, and cholesteryl anthraquinone derivatives, organogels, or any combinations thereof.

In some embodiments, the composition (e.g., the base or vehicle) can include an amount of DMDM Hydantoin (e.g., CAS No: 6440-58-0), which can be a preservative (e.g., an antimicrobial formaldehyde releaser preservative). DMDM Hydantoin can cause formaldehyde allergy in some cases, and can be omitted. Other preservatives can be used, such as naticide, radish root, Tetrasodium EDTA, Tetrahydrate, Methyl Paraben, Optiphen, Phenoxyethanol, Potassium Sorbate, Propyl Paraben, Tetrasodium Glutamate Diacetate, phenoserve, or any combinations thereof.

In some embodiments, the composition can include an amount of a humectant that has low comedogenicity. Glycerin (e.g., 0/5 comedogenicity), Sodium PCA (e.g., 0/5 comedogenicity), dimethicone (e.g., 2%), c30-45 alkyl methicone, c30-45 alkyl olefin, microcrystaline wax, polyethylene, silica dimethyl silylate, or combinations thereof can be used in the composition, for example as a humectant.

In some embodiments, the composition can include a PEG-free water-in-silicone or a water-in-oil silicone emulsifier (e.g., Cetyl diglyceryl tris(trimethylsiloxy)silylethyl dimethicone). In some cases, a penetration enhancer can be used that has dual effects, such as an analgesic or other therapeutic effect as well as enhancing penetration. The penetration enhancer can have low activation of the TRPV receptor. The penetration enhancer can have no aversive smell and/or no burning sensation. For a penetration enhancer, the composition can include one or more of Terpenes, Terpenoids, menthol (e.g., about 0.25% to about 20%, or any values or ranges therebetween), Camphor (e.g., about 0.25% to about 20%, or any values or ranges therebetween); Limonene (e.g., d-limonene) (e.g., about 0.25% to about 20%, or any values or ranges therebetween), Myrcene, Pinene (e.g., a-pinene, B-Pinene), p-Cymene, Carvone, Borneol (e.g., about 0.25% to about 20%, or any values or ranges therebetween), Labrasol, Transcutol, a derivative from Cannabis plant, a-humulene, Linalool, Myrcene, Terpinolene, a-Bisabolol, B-Caryophyllene, Nerolidol, B-Caryophylle, Eugenol, CBD, Cannabis derivatives, non-terpene penetration enhancers, dimethyl sulphoxide, acetone, propylene glycol, and tetradihydrofuryl alcohol, or any combination thereof. For penetration enhancers that also have a therapeutic effect (e.g., analgesic), the sugar (e.g., dextrose) and/or sugar alcohol (e.g., mannitol) can enhance the therapeutic effect (e.g., as discussed herein), and the penetration enhancer can also enhance the effect of the sugar (e.g., dextrose) and/or sugar alcohol (e.g., mannitol), with a reciprocal functional enhancement.

In some embodiments, the composition (e.g., the base or vehicle) can include a viscosity modifier, which can include an amount of hyaluronic acid (e.g., CAS No: 9004-61-9). Hyaluronic acid can be included with a humectant to liberate perineural space by disassociating fluid in connective tissue space, which surrounds nerves, thereby reducing mechanical irritation of the nerve. In some cases, the composition can include a therapeutic agent and an agent that changes a mechanical or physical property of the tissue to have simultaneous chemical and mechanical effects. In some cases, an amount of hyaluronic acid can be used as a humectant for the composition, as discussed herein.

The composition (e.g., the base or vehicle) can include an emollients, which can be used to soften and/or smooth the skin. The emollient can include an occlusive agent or effect, such as to provide a layer of protection that helps prevent moisture loss from the skin. Some example emollients that can be used for the composition include silicone (e.g., dimethicone, cyclomethicone), vegetable oils (grape seed, sesame seed, jojoba, etc.), butters (cocoa butter, shea butter), alcohols (stearyl alcohol, cetyl alcohol), and petrolatum derivatives (petroleum jelly, mineral oil), or any combinations thereof.

A humectant is a substance that actually bonds with water molecules to increase the water content in the skin itself. As discussed herein, the composition can include an amount of one or more humectants. Some humectants also have emollient properties, while not all emollients are humectants. Various ingredients can be used for the composition to provide moisturizing effects including proteins, acids, polysaccharides, and various small molecules (e.g. glycerine, sorbitol, urea, aloe vera etc.). The TRPV-4 receptor can be triggered by dehydration and higher osmotic concentrations. Increasing the moisture in the tissue can aid in calming the nerve.

In some embodiments, the composition can include multiple therapeutic ingredients. In some cases, one or more therapeutic ingredients can complement or enhance the therapeutic effect(s) of one or more of the other therapeutic ingredients. The resulting therapeutic effect can be synergistic, having a combined therapeutic effect that is greater than the combination of the therapeutic effect of the individual components. For example, the sugar, sugar alcohol, or alkalizing agent, or any combination thereof, can enhance the analgesic effect of certain analgesic substances, when also included in the composition. The alkalizing agent can enhance the analgesic effect, in addition to or without the sugar or sugar alcohol. For example, the efficacy or potency of the analgesic can be improved by the sugar, sugar alcohol, or alkalizing agent, or any combination thereof. The analgesic effect can be extended or prolonged by the sugar, sugar alcohol, or alkalizing agent, or any combination thereof. Without being limited by theory, it is believed that the sugar (e.g., glucose) or sugar alcohol and/or the alkalizing agent can facilitate mitochondrial function and can increase uptake of the therapeutic agent. Elevated sugar (e.g., glucose) concentration can increase endothelial permeability, which can improve update of topical analgesics and other therapeutics. The improved therapeutic effects can enable the composition to have lower concentrations of the active ingredients, can enable effective therapy with smaller amounts of the composition, can result in lower rates or degrees of side effects, such as less skin irritation and less load on the systemic metabolism.

In some embodiments, the composition can include an amount of menthol as well as one or more of: i) an amount sugar; ii) an amount of sugar alcohol, and iii) an amount of an alkalizing agent. The composition can include various other components, as discussed herein, such as a vehicle or base and other therapeutic agents. Menthol can be an analgesic, and the sugar, sugar alcohol, and/or alkalizing agent can enhance the analgesic effect of the menthol, as discussed herein. The amount of menthol can be between about 0.1% and about 25% by weight of the composition, between about 0.1% and about 5%, or between about 0.1% and about 1.5% by weight of the composition. The amount of menthol can be about 0.1%, about 0.2%, about 0.3%, about 0.5%, about 0.7%, about 1%, about 1.25%, about 1.5%, about 1.75%, about 2%, about 2.5%, about 3%, about 3.5%, about 4%, about 5%, about 7% about 10%, about 15%, about 20%, or about 25% by weight of the composition, or any ranges or value therebetween, although other amounts could be used. Menthol can also be a used as a penetration enhancer, which can enhance the effectiveness of the sugar, sugar alcohol, and/or alkalizing agent (e.g., by reciprocal functional enhancement).

In some embodiments, the composition can include an amount of camphor as well as one or more of; i) an amount sugar; ii) an amount of sugar alcohol, and iii) an amount of an alkalizing agent. The composition can include various other components, as discussed herein, such as a vehicle or base and other therapeutic agents. Camphor can be an analgesic, and the sugar, sugar alcohol, and/or alkalizing agent can enhance the analgesic effect of the camphor, as discussed herein. The amount of camphor can be between about 0.1% and about 25% by weight of the composition, between about 0.1% and about 5%, or between about 0.1% and about 1% by weight of the composition. The amount of camphor can be about 0.1%, about 0.2%, about 0.3%, about 0.5%, about 0.7%, about 1%, about 1.25%, about 1.5%, about 1.75%, about 2%, about 2.5%, about 3%, about 3.5%, about 4%, about 5%, about 7% about 10%, about 15%, about 20%, or about 25% by weight of the composition, or any ranges or value therebetween, although other amounts could be used. Camphor can also be used as a penetration enhancer, which can enhance the effectiveness of the sugar, sugar alcohol, and/or alkalizing agent (e.g., by reciprocal functional enhancement).

In some embodiments, the composition can include an amount of a TOR- or m-TOR-modulator, for example a phenol (e.g., CAS No: 108-95-2), a polyphenol, or a phenolic compound as well as one or more of: i) an amount sugar; ii) an amount of sugar alcohol, and iii) an amount of an alkalizing agent. The composition can include various other components, as discussed herein, such as a vehicle or base and other therapeutic agents. Phenol, polyphenols, and phenolic compounds can be an analgesic, can promote sirtuin production in cells, and can be particularly beneficial for neuropathy therapy. The sugar, sugar alcohol, and/or alkalizing agent can enhance the therapeutic effect of the TOR- or m-TOR-modulating compound, phenol, polyphenol, and/or phenolic compounds, as discussed herein. The combination of the phenol, polyphenol, and/or phenolic compounds, including fruit or plant extracts with high-phenol content, with the sugar, sugar alcohol, and/or alkalizing agent can produce synergy because the phenol, polyphenol, and/or phenolic compounds work with the mitochondria, and it is believed that the sugar, sugar alcohol, and/or alkalizing agent can improve mitochondrial function. The phenol, polyphenol, and/or phenolic compounds, or TOR- or m-TOR-modulator can be included with a concentration of about 0.1% to about 25% be total weight or volume of the composition, or about 20-2,500 mg/ml.

In some embodiments, the composition can include an amount of a salicylate as well as one or more of: i) an amount sugar; ii) an amount of sugar alcohol, and iii) an amount of an alkalizing agent. The salicylate can include wintergreen, trolamine salicylate (e.g., CAS No: 2174-16-5), etc. The composition can include various other components, as discussed herein, such as a vehicle or base and other therapeutic agents. The salicylate can be an analgesic, and the sugar, sugar alcohol, and/or alkalizing agent can enhance the analgesic effect, as discussed herein. The amount of the salicylate can be between about 0.1% and about 25% by weight of the composition, between about 0.1% and about 5%, or between about 0.1% and about 1.5% by weight of the composition. The amount of the salicylate can be about 0.1%, about 0.2%, about 0.3%, about 0.5%, about 0.7%, about 1%, about 1.25%, about 1.5%, about 1.75%, about 2%, about 2.5%, about 3%, about 3.5%, about 4%, about 5%, about 7% about 10%, about 15%, about 20%, or about 25% by weight of the composition, or any ranges or value therebetween, although other amounts could be used.

In some embodiments, the composition can include an amount of one or more of: a) allantoin, b) aluminium acetate, c) sodium bicarbonate, d) trolamine, e) zinc oxide, f) zinc acetate, g) juniper tar, h) butambem picrate, or any combination thereof, which can have therapeutic (e.g., analgesic) effects. The composition can also include one or more of: i) an amount sugar; ii) an amount of sugar alcohol, and iii) an amount of an alkalizing agent. The composition can include various other components, as discussed herein, such as a vehicle or base and other therapeutic agents. The sugar, sugar alcohol, and/or alkalizing agent can enhance the therapeutic (e.g., analgesic) effects, as discussed herein. By way of example, the composition can include about 5-40% sugar or sugar alcohol, about 0.5-15% alkalizing agent, and about 0.5-30% of other therapeutic agents, with the remainder as a base or vehicle.

In some embodiments, the composition can include an amount of a -caine (e.g., a synthetic alkaloid anesthetic such as lidocaine or benzocaine) as well as one or more of: i) an amount sugar; ii) an amount of sugar alcohol, and iii) an amount of an alkalizing agent. The composition can include various other components, as discussed herein, such as a vehicle or base and other therapeutic agents. The lidocaine (or other -caine) can be an analgesic, and the sugar, sugar alcohol, and/or alkalizing agent can enhance the analgesic effect, as discussed herein. The amount of the lidocaine (or other -caine) can be between about 0.5% and about 10% by weight of the composition, for example. The amount of the lidocaine (or other -caine) can be about 0.5%, about 0.7%, about 1%, about 1.25%, about 1.5%, about 1.75%, about 2%, about 2.5%, about 3%, about 3.5%, about 4%, about 5%, about 7%, or about 10% by weight of the composition, or any ranges or value therebetween, although other amounts could be used. These same concentrations can also apply to benzocaine in the composition.

In some embodiments, the composition can include an amount of a hydrocortisone or hydrocortisone acetate as well as one or more of; i) an amount sugar; ii) an amount of sugar alcohol, and iii) an amount of an alkalizing agent. The composition can include various other components, as discussed herein, such as a vehicle or base and other therapeutic agents. The hydrocortisone or hydrocortisone acetate can be an analgesic, and the sugar, sugar alcohol, and/or alkalizing agent can enhance the analgesic effect, as discussed herein. The amount of the hydrocortisone or hydrocortisone acetate can be between about 0.2% and about 10% by weight of the composition, for example. The amount of the hydrocortisone or hydrocortisone acetate can be about 0.2%, about 0.3%, about 0.5%, about 0.7%, about 1%, about 1.25%, about 1.5%, about 1.75%, about 2%, about 2.5%, about 3%, about 3.5%, about 4%, about 5%, about 7%, or about 10% by weight of the composition, or any ranges or value therebetween, although other amounts could be used.

In some embodiments, the composition can include an amount of an opioid as well as one or more of: i) an amount sugar; ii) an amount of sugar alcohol, and iii) an amount of an alkalizing agent. The composition can include various other components, as discussed herein, such as a vehicle or base and other therapeutic agents. The opioid can be an analgesic, and the sugar, sugar alcohol, and/or alkalizing agent can enhance the analgesic effect, as discussed herein, which can result is less of the opioid being used, which can be beneficial. By way of example, the composition can include about 5-400% sugar or sugar alcohol, about 0.5-15% alkalizing agent, about 0.5-30% of other therapeutic agent. One embodiment can include about 10 mg/ml of morphine.

The therapeutic composition can include, and can enhance the effectiveness of, an analgesic such as ibuprofen or other nonsteroidal anti-inflammatory drugs (NSAIDs), anti-inflammatory drugs (e.g., including steroids), fragments of naturally occurring hormones and peptides, CGRP-inhibitors or agents that specifically counteracts CGRP (calcitonin gene-related peptide), stem cells, amniotic or embryonic derivative, a hormonal (e.g. pro-progesterone or anti-estrogen activity) or immune system modulator (e.g. anti-bodies), or combinations thereof.

In some embodiments, the composition can include an amount of platelet-rich plasma (PRP), which can also be known as autologous conditioned plasma (ACP), as well as one or more of: i) an amount sugar; ii) an amount of sugar alcohol, and iii) an amount of an alkalizing agent. The composition can include various other components, as discussed herein, such as a vehicle or base and other therapeutic agents. PRP can be applied topically to facilitate tissue turnover and tissue repair, and PRP can also have analgesic effects. The sugar, sugar alcohol, and/or alkalizing agent can enhance the therapeutic effects of PRP, as discussed herein, and the composition can provide a substrate for regional mitochondria to facilitate the tissue turnover and/or repair. By way of example, the composition can include about 5-40% sugar or sugar alcohol, about 0.5-15% alkalizing agent, about 10-50% vehicle, and about 0.5-30% other therapeutic agent(s) (e.g., PRP). The PRP can be formed by obtaining whole blood (e.g., drawn from the patient or subject receiving the treatment) and centrifuging the whole blood to remove red blood cells or other blood contents, or otherwise increasing the concentration of platelets in the blood. The PRP can be applied topically, e.g., to the face or other skin. In some cases the PRP can be applied topically using micro-needles or nano-needles to facilitate penetration. The PRP can be mixed or otherwise combined with the other components of the composition as discussed herein. In some cases, the PRP can be used for an initial treatment (e.g., without the full composition), and the excess or residue PRP can be collected and combined with a composition base to form the full composition that includes PRP. A subject can receive an initial PRP treatment (e.g., at a provider's facility or office) and can take the composition containing PRP home for further treatment (e.g., which can be self-administered). In some cases, the PRP can be combined with the other components of the composition without an initially PRP treatment. The PRP can be combined with sugar, sugar alcohol, or alkalizing agent before being used on the subject.

In some embodiments, the composition can include an amount of one or more peptides, as well as one or more of: i) an amount sugar; ii) an amount of sugar alcohol, and iii) an amount of an alkalizing agent. The composition can include various other components, as discussed herein, such as a vehicle or base and other therapeutic agents. The amount of peptide can be therapeutic as an analgesic and/or can have healing effects. The sugar, sugar alcohol, and/or alkalizing agent can enhance the therapeutic effects of the amount of peptide(s), as discussed herein. Also, the adjusted pH (e.g., alkalinity) can help preserve the peptides. The amount of peptide(s) can include AOD9604 (e.g., CAS No: 221231-10-3). In some embodiments, the composition can include AOD9604 and hyaluronic acid (HA). The composition can include peptide fragments, such as amino acids 176-191 of the human growth hormone polypeptide. The composition can include one or more fragments of growth hormone molecule (e.g., amino acids), which can optionally be in combination with hyaluronic acid. The composition can include pentadecapeptide BPC 157 (e.g., CAS No: 137525-51-0), which can be a partial sequence of body protection compound (BPC), and which can help with healing of tendons, wounds, tendon-to-bone tissue, and can decrease pain such as in sprains, tears etc. The composition can include about 1.5 mg, about 1.75 mg, about 2 mg, about 2.25 mg, or about 2.5 mg of pentadecapeptide BPC 157 per ml of the composition, or any values or ranges therebetween, for example. The composition can include cerebrolysin (e.g., CAS No: 12656-61-0), or fragments of cerebrolysin. The composition can include follistatin 344, such as about 0.5 mg, about 0.75 mg, about 1 mg, about 1.25 mg, or about 1.5 mg per ml of the composition, or any ranges or values therebetween. The composition can include pentosan polysulfate (e.g., CAS No: 37300-21-3) or oversulphated glycosaminoglycan. The composition can include an amount of one or more melanocortins (e.g., about 0.25 mg, about 0.5 mg, or about 0.75 mg per ml of the composition, or any values or ranges therebetween). The composition can include an amount of one or more thymosins (e.g., about 3 mg per ml of the composition). The composition can include vasoactive intestinal polypeptide (VIP) (e.g., CAS No: 69698-54-0), such as about 0.25 mg, about 0.5 mg, or about 0.75 mg per ml of the composition. The composition can include thymulin (e.g., CAS No: 63958-90-7). The composition can include about 0.1 mg, about 0.2 mg, about 0.3 mg, about 0.5 mg, about 0.75 mg, about 1 mg, about 1.25 mg, about 1.5 mg, about 1.75 mg, about 2 mg, about 2.5 mg, about 3 mg, about 3.5 mg, about 4 mg, about 4.5 mg, or about 5 mg of the peptide(s) per ml of the composition, or any values or ranges therebetween, although other amounts can be used in some cases.

In some embodiments, the composition can include a TOR- or m-TOR-modulating compound, for example a phenol (e.g., CAS No: 108-95-2) or a polyphenol as well as one or more of: i) an amount sugar; ii) an amount of sugar alcohol, and iii) an amount of an alkalizing agent. The composition can include various other components, as discussed herein, such as a vehicle or base and other therapeutic agents. The TOR- or m-TOR modulating compound, phenol or polyphenol can have an analgesic effect, and the sugar, sugar alcohol, and/or alkalizing agent can enhance the analgesic effect, as discussed herein (e.g., by facilitating mitochondrial metabolism). The composition can include resveratrol (e.g., CAS No: 501-36-0) or its analogs, quercetin (e.g., CAS No: 117-39-5), pterostilbene (e.g., CAS No: 53742-8), or eugenol (e.g., CAS No: 97-53-0), or any combinations thereof. Alternatively, the composition might include a fruit or plant extract as a means of supplying these elements in a more stable manner—a preferred embodiment. By way of example, about 5-500 mg of the analogs and/or around 20-2,500 mg/ml of the fruit extracts can be used.

In some embodiments, the composition can include one or more flavonoids, as well as one or more of; i) an amount sugar; ii) an amount of sugar alcohol, and iii) an amount of an alkalizing agent. The composition can include various other components, as discussed herein, such as a vehicle or base and other therapeutic agents. The sugar, sugar alcohol, and/or alkalizing agent can enhance the therapeutic effects of the flavonoids, as discussed herein. For example, the composition can include one or more flavones (e.g., Ginkgo biloba, luteolin, tangeritin, and/or sinensetint), one or more isoflavonoids (e.g., genistein and/or daidzein), one or more flavanones (e.g., hesperitin, naringenin, and/or eriodictyol), one or more flavonols (e.g., quercetin, myricetin, and/or fisetin), one or more neoflavonoids, one or more flavanonols (e.g., catechins), or any combination thereof. By way of example, the composition can include about 5-40% sugar or sugar alcohol, about 0.5-15% alkalizing agent, about 0.5-30% of other therapeutic agent, be weight or volume.

In some embodiments, the composition can include one or more olligostillbenoids, stilbenes, flavonoids, sirtuin promoters, NAD+ promoters (e.g. nicotinamide riboside chloride), or any combinations thereof, together with one or more of: i) an amount sugar; ii) an amount of sugar alcohol, and iii) an amount of an alkalizing agent. The composition can include various other components, as discussed herein, such as a vehicle or base and other therapeutic agents. The sugar, sugar alcohol, and/or alkalizing agent can enhance the therapeutic effects (e.g., analgesic) of the above-identified components, as discussed herein. By way of example, the composition can include about 5-40% sugar or sugar alcohol, about 0.5-15% alkalizing agent, about 0.5-30% of other therapeutic agent, be weight or volume.

In some embodiments, the composition can include one or more Cannabis derivatives, such as Cannabis-derived terpenes, Cannabis-derived chemvars, cannabinoids (which can be a class of mono- to tetracyclic C21 (or C22) meroterpenoids, Cannabitriol (CBT), Cannabinol (CBN), Cannabigerol (CBG)), cannabidiol (CBD), cannabinol, cannabinoid acids, cannabigerol, cannabivarins, the monoterpenoids myrcene, limonene, pinene, the sesquiterpenoid β-caryophyllene, or phytocannabinoids, or any combination thereof together with one or more of; i) an amount sugar; ii) an amount of sugar alcohol, and iii) an amount of an alkalizing agent. The composition can include various other components, as discussed herein, such as a vehicle or base and other therapeutic agents. The sugar, sugar alcohol, and/or alkalizing agent can enhance the therapeutic effects (e.g., analgesic) of the above-identified components, as discussed herein.

Applicator

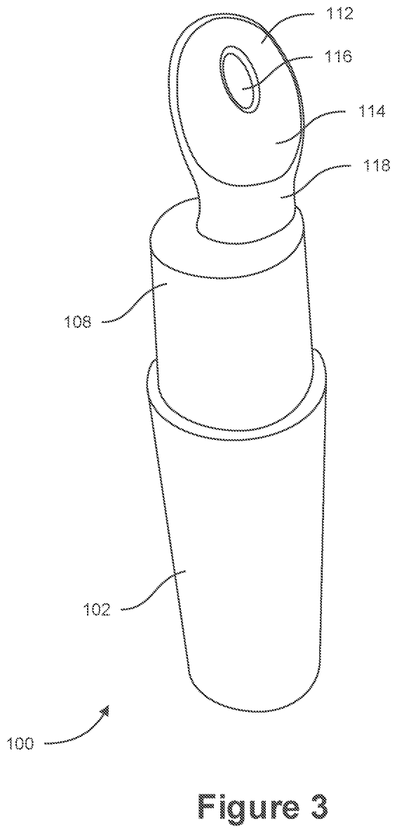

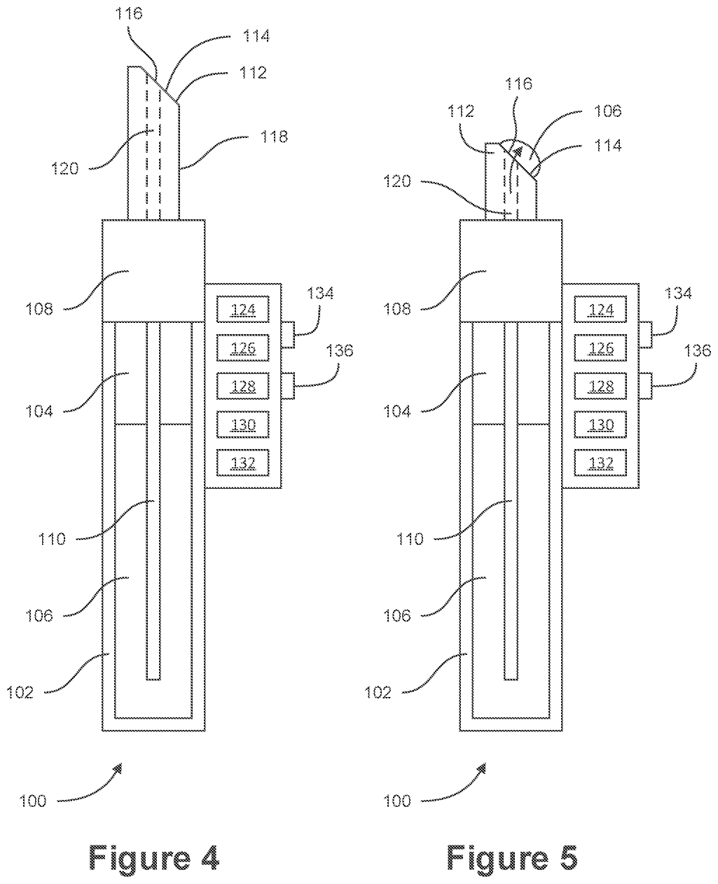

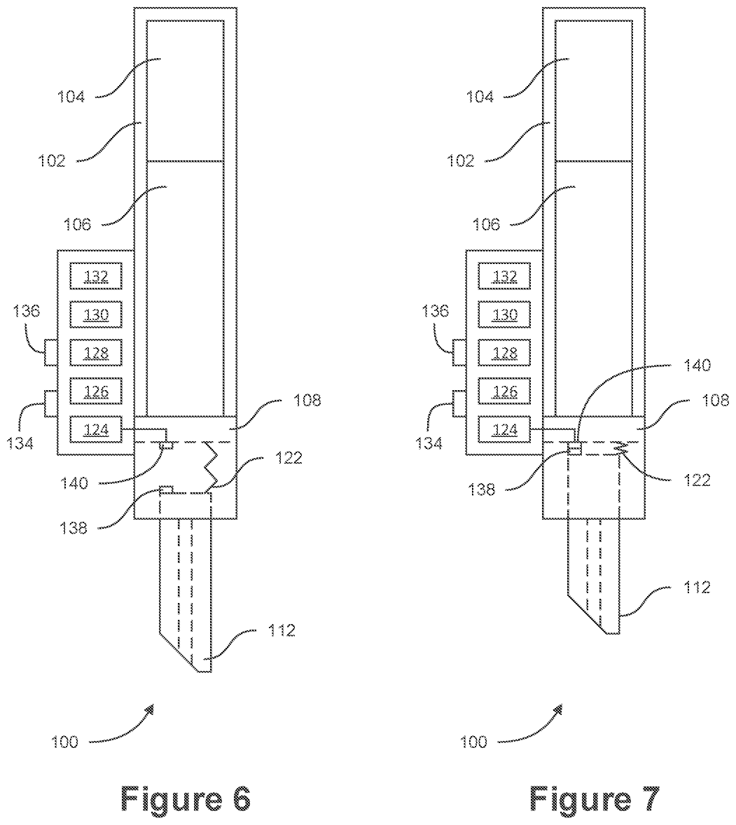

With reference to FIG. 3, an applicator 100 can be used to administer a therapeutic composition, such as those disclosed herein. The applicator 100 can include a housing 102 or shell that defines an internal cavity 104, which can hold an amount of the composition 106. The applicator 100 can include a dispenser 108, which can be configured to dispense amounts of the composition 106. FIGS. 4 and 5 are schematic, side views of an example embodiment of an applicator 100. In some cases, the dispenser 108 can be a pump, although other types of dispensers can be used, as discussed herein. The applicator 100 can include a draw tube 110, which can extend from the dispenser 108 into the internal cavity 104. When the dispenser 108 (e.g., pump) is actuated, an amount of the composition 106 can be drawn into the draw tube 110, which can drive an amount of the composition 106 out through the dispenser 108, as shown for example in FIG. 5. The housing 102 can be firm. In some embodiments, the draw tube 110 can be omitted. For example, the applicator 100 can be designed to be operated with the dispenser 108 below the housing 102, as shown in FIG. 6, for example, so that gravity can pull the composition 106 towards the dispenser 108.

The applicator 100 can include a contact tip 112. The applicator 100 can be configured so that the dispenser 108 can dispense an amount of the composition 106 onto the contact tip 112, as shown for example in FIG. 5. The contact tip 112 can be configured to enable a user to manipulate tissue to liberate nerves. By way of example, when a nerve becomes inflamed or irritated, it can become sticky and can stick to the surrounding tissue. Manipulating the nerve and/or surrounding tissue, such as by pressing, rubbing, massaging, kneading, and the like, can separate the nerve from the surrounding tissue, thereby liberating the nerve. The contact tip 112 can be configured to facilitate the tissue manipulation to liberate nerves. For example, the user can pressing the contact tip against the skin and can move the contact tip 112 while applying pressure to manipulate the tissue and liberate the nerve. The contact tip 112 can be firm or rigid. In some cases, the contact tip 112 can be flat, for example, as shown in FIGS. 3 to 6. In some cases, the contact tip 112 can have a contact surface 114 (e.g., substantially flat) that surrounds an opening 116. The contact surface 114 can be configured to press or rub against the skin or other tissue, such as to apply the composition and/or to manipulate the tissue for nerve liberation. The applicator 100 can dispense the composition through the opening 116 and onto the contact tip 112, such as onto the contact surface 114. A shaft 118 extend from the contact surface 112 toward the housing 102. The shaft 118 can elevate or separate the contact surface 112 away from the housing 102. A conduit 120 can extend from the opening 116 towards the housing 102 so that the composition 106 can be dispensed from the internal cavity 104, through the conduit 120, through the opening 116, and onto the exterior of the contact tip 112 (e.g., onto the surface 114). The contact surface 114 can be wider than the shaft 118. The wide and flat contact surface 114 coupled to a thinner shaft can form a spatula tip configuration.

In some embodiments, the contact tip 112 can move relative to the housing 102 to dispense the composition 106. For example, FIG. 4 shows the contact tip 112 in a neutral position, and FIG. 5 shows the contact tip 112 in a depressed position. When the contact tip 112 moves from the neutral position to the depressed position, at least a portion of the shaft 118 can be driven into the dispenser 108 (e.g., pump) or housing 102. Depressing the contact tip 112 can actuate the pump or other dispenser 108 to drive an amount of the composition 106 out of the applicator 100, such as onto the contact tip 112 as shown in FIG. 5. The contact tip 112 can be biased towards the neutral position, such as by a spring 122 or other biasing member.

The user can dispense an amount of the composition 106 onto the contact tip 112 and can press and/or rub the contact tip 112 against the skin, which can facilitate penetration of the composition 106 into or through the skin and can also liberate the underlying nerve(s). The applicator 100 can be used to combine the manual liberation of the nerve with the therapeutic topical treatment of the composition, which can include, for example, sugar, sugar alcohol, one or more alkalizing agents, and/or the various other therapeutic components and combinations disclosed herein. The combination of the chemical component of the treatment (e.g., from the therapeutic composition) and the mechanical component of the treatment (e.g., the mechanical nerve liberation) can be synergistic. The composition can be significantly more effective on a liberated nerve.

By using the applicator 100 to apply the composition 106, the user can avoid touching the composition 106. The skin of the hand, especially the palm, can be very absorptive. So using the applicator 100 instead of a hand to apply the composition 106 can result in more of the composition being absorbed into the target area, as compared to applying the composition using a hand that can absorb the composition. Also, for some embodiments of the composition 106, it can be advantageous to avoid contacting the composition to skin that is not the target for the treatment, such as if the composition has a undesirable smell, can stain, has numbing effects, or has side effects that a user may want to avoid. For example, if the composition has numbing effects, that can be beneficial for treating the painful target area, but numbing of the hands may be undesirable.

The contact surface 114 can be smooth or otherwise configured to enable the contact tip 112 to glide over the skin. This can be beneficial for certain types of tissue manipulation that can be useful for nerve liberation, such as sliding the tip 112 along a nerve pathway while applying pressure. For example, a low-friction material can be used, and in some cases, the composition 106 can be serve as a lubricant to facilitate sliding of the contact tip 112 across the skin. In some cases, the contact surface 114 can be configured to grip the skin, so as to prevent the contact tip 112 from sliding across the skin. For example, the contact surface 114 can include rubber or another high-friction material. The contact surface 114 can include texture or gripping features, such as an array or raised bumps, to impede the contact tip from sliding across the skin. The texture or gripping features can facilitate gripping against the skin when the composition 106 is present, since the composition 106 can lubricate the skin in some instances. The gripping contact tip 112 can be beneficial for certain types of tissue manipulation that can be useful for liberating nerves. For example, a user can press the contact tip 112 against the skin to anchor the contact tip 112 into the tissue. The user can then move the contact tip 112, such as in small circular motions, to pull the surface of the skin. In some embodiments, the contact tip 112 can be configured to slide over the skin, while in other embodiments the contact tip 112 can be configured to grip against the skin. In some cases, the contact tip 112 can have a first portion (e.g., a first area of the contact surface 114) that is configured to slide across the skin, and a second portion (e.g., a second area of the contact surface 114) that is configured to grip against the skin. The two areas can be upper and lower areas, or left and right areas, for example. The user can tilt the applicator so that that more pressure is applied to the first portion of the contact tip 112 in order to slide the contact tip 112 across the tissue, and the user can tilt the applicator so that more pressure is applied to the second portion of the contact tip 112 in order to anchor the contact tip 112 into the tissue.

In some embodiments, the contact tip 112 can be configured to apply stimulation to the nerve. For example, the contact tip 112 can be electrified so as to provide electrostimulation to the nerve. The contact tip 112 can be coupled to a vibrator so as to provide vibratory stimulation to the nerve. The contact tip 112 can be coupled to a heating device so that the contact tip 112 can be heated to provide caloric stimulation to the nerve. The contact tip 112 can be coupled to a cooling device, which can cool the contact tip 112, which can stimulate nerves. In some cases, cooling the tissue can calm the nerve and produce an analgesic effect. A combination of multiple stimulus can be used, in some cases.

The applicator 100 can include a power source 126, such as a battery (e.g., replaceable or rechargeable), which can provide electrical energy to electrical components of the applicator 100, such as a controller 124, a vibrator 128, a heating device 130, a cooling device 132, one or more user input elements 134, one or more notification or output element 136, or any combinations thereof. The power source 126 can provide electricity to the contact tip 112 so that the contact tip 112 can deliver the electricity to the subject to provide electrostimulation to the nerve. The controller 124 can control the electricity delivered to the contact tip 112. The electricity can be delivered in pulses, waves, or as a steady state amplitude over a time, or any combinations thereof. The contact tip 112 can be electrically conductive. For example, a metal contact tip 112 can be used, although other electrically conductive materials can also be used. The composition 106 can be electrically conductive. Electrical current can be delivered from the power source 126, through the contact tip 112, through the composition 106 in some instances, and to the subject for electrostimulation. The electrical stimulus can be of a frequency that assists in driving therapeutic chemical compound deeper into the tissue. One example uses brief, local, high voltage pulses (e.g., 50-500 volts) for short times of up to one second to increase transport across the skin through electroporation, which is a technique that temporarily and reversibly creates channels across the epidermal layer allowing for deeper passage of therapeutic agents.

The applicator 100 can include a vibrator 128, which can be mechanically coupled to the contact tip 112 so that the vibrator 128 can vibrate the contact tip. The controller 124 can control the vibrator 128, such as to set or vary the frequency and/or amplitude of the vibration. The frequency of the vibration can be steady or varied, such as in waves or steps. The amplitude of the vibration can be steady or varied, such as in waves or pulses. The vibration can be delivered to the subject via the contact tip 112 to perform vibratory nerve stimulation. In some embodiments, the vibrator can be used to facilitate mechanical nerve liberation. For example, the vibrator 128 can vibrate the contact tip 112 as the user manipulates the tissue with the vibrating contact tip 112. The vibrations can further facilitate liberation of the nerve. The contact tip can move laterally, such as to perform an abrasive action (e.g., like sand paper), and the lateral motion can be linear, circular, or any other suitable pattern. The contact tip can have a longitudinal, stamping motion, which can puncture into the skin. The user interface, can enable the user to select between different types of motion for the vibration. The user interface can enable the user to adjust the frequency and/or intensity of the vibration.

The applicator 100 can include a heating element 130, which can be thermally coupled to the contact tip 112 so that the heating element can heat the contact tip 112. The controller 124 can control the temperature or heating of the contact tip 112. In some cases, the applicator 100 can include a temperature sensor, which can be used to measure or determine a temperature, such as of the contact tip 112. The controller 124 can use the temperature for feedback control, for example, to operate the heating element 130 to produce target temperature(s). The temperature or heating can be controlled by the controller 124 to be steady or varied, such as in waves or pulses. The heating can be applied through the contact tip 112 to the subject, for caloric stimulation of the nerve. Heat can also facilitate liberation of the nerve.

The applicator 100 can include a cooling element 132, which can be thermally coupled to the contact tip 112 so that the cooling element 132 can cool the contact tip 112. The controller 124 can control the temperature or cooling of the contact tip 112. As mentioned above, the applicator 100 can include a temperature sensor, which can be used to measure or determine a temperature, such as of the contact tip 112. The controller 124 can use the temperature for feedback control, for example, to operate the cooling element 132 to produce target temperature(s). The temperature or cooling can be controlled by the controller 124 to be steady or varied, such as in waves or pulses. The cooling can be applied through the contact tip 112 to the subject, for cooling stimulation of the nerve. In some cases, cooling of the tissue can calm the nerve and provide an analgesic effect. In some cases, a combined heating and cooling device can be used, or separate heating and cooling devices can be used, or the applicator 100 can include only one of a heating element 130 or a cooling element 132. A Peltier device can be used for heating and/or cooling, in some embodiments. A thermoelectric cooler and/or heater can be used. The contact tip can be thermally conductive.

Any combination of electrical current, vibration, heating, and cooling can be applied, either in series or in parallel. For example, the electrical current and vibration can be applied together simultaneously. The applicator can apply electrical current during a first time and vibration during a second time, and in some cases that pattern can repeat. Heating or cooling can be applied before or after or simultaneously with the vibration and/or electrical current.

The applicator 100 can have one or more user input elements 134, which can receive input from a user. The user input element(s) 134 can include one or more buttons, switches, dials, touchscreens, and the like. The controller 124 can control the applicator 100 based on input received by the one or more user input elements 134. For example, the user can push a button to turn the electrical current, vibration, heating, and/or cooling on or off. The user can provide input to specify parameters (e.g., frequency and/or amplitude) of the electrical current, vibration, heating, and/or cooling or to select between available treatment profiles, which can be stored in computer readable memory. The different treatment profiles can include instructions that the controller can execute to implement different combinations or parameters of the electrical current, vibration, heating, and/or cooling.

The applicator 100 can include one or more user notification or output elements 136, which can output information or notifications to the user. For example, the one or more user notification or output elements 136 can include one or more lights, audio speakers, a vibrator, a display, and the like. The controller 124 can operate the one or more user notification or output elements 136 to convey information to the user. For example, the controller 124 can implement a timer, which can start when the user begins the treatment for a location. The timer can be started based on input from the user (e.g., pressing a button), from depressing of the contact tip 112, from dispensing of the composition 106, etc. When a predetermined period of time has passed, the controller 124 can output a notification to the user (e.g., by flashing a light or vibrating the applicator 100 in a notable manner). The notification can be a signal to the user to end the treatment or to move the treatment to a next location. The timer can last for 10 seconds, 15 seconds, 20 seconds, 25 seconds, 30 seconds, 35 seconds, 40 seconds, 45 seconds, 50 seconds, 55 seconds, 60 seconds, 70 seconds, 80 seconds, 90 seconds, 100 seconds, 110 seconds, 120 seconds, or more, or any values or ranges therebetween, although other times could be used for certain therapies. The one or more user output elements 136 can be used to convey other information, such as a low battery, a malfunction, a selected treatment profile or parameter, etc.

In some cases, the same vibrator 128 can be used for vibrating the contact tip 112, as discussed herein, and for providing information to the user, or separate vibrators can be used. The vibrator 128 can be mechanically coupled to the housing 102 or other portion of the applicator 100 that is configured to be gripped by the user, so that that the vibrator 128 can provide a vibration felt the user (e.g., through the hand that holds the applicator 100). The notification or informational vibration can be different from the treatment vibration, so that the user can distinguish between vibrations that are part of the treatment and vibrations that convey information. For example, the vibration for treatment can be at a steady state frequency and amplitude, whereas the notification vibration can be one or more pulses of vibration at a different frequency or amplitude. The notification vibrations can have a higher amplitude than the treatment vibrations. In some cases, the vibration can stop to convey information. For example, when the timer reaches the threshold time, the vibration can stop, which can instruct the user to stop the therapy or move the therapy to a new location. The vibration can stimulate nerves and provide therapeutic afferent signals to the CNS, can facilitate liberation of provocative tissue surround nerves, and can also drive therapeutic agent into deeper tissues (e.g., into perineural space to affect symptomatic nerves).

With reference to FIGS. 6 and 7, the applicator 100 can have a contact tip depression sensor that can determine whether the contact tip 112 is at the neutral position (e.g., FIG. 6) or the depressed position (e.g., FIG. 7). A first electrical contact 138 (e.g., a pin or pad) can be coupled to the contact tip 112, and a second electrical contact 140 can be coupled to the housing 102 or dispenser 108 so that the first electrical contact 138 move towards the second electrical contact 140 as the contact tip 112 is moved from the neutral position to the depressed position. When the contact tip 112 is at the depressed position, as shown in FIG. 7, the first electrical contact 138 can touch the second electrical contact 140, which can generate a signal, which can be delivered to the controller 124. The signal can be an indication that the contact tip 112 is fully depressed. Touching the first electrical contact 138 to the second electrical contact 140 can complete an electrical connection that generates the signal, for example. Any suitable approach can be used to generate a signal when the contact tip 112 is moved to the depressed position.

The controller 124 can take action in response to the signal indicating that the contact tip 112 has moved to the depressed position. For example, the controller 124 can start a timer, as discussed herein, in response to the signal. The controller 124 can initiate electrostimulation, a vibrator 128, a heating element 130, and/or a cooling element 132, as discussed herein, in response to the signal. The controller 124 can electrify the contact tip 112, vibrate the contact tip 112, heat the contact tip 112, and/or cool the contact tip 112 in response to the signal. The controller 124 can output information to the user in response to the signal, such as via the user output or notification element 136, such as by a sound, a light, or a vibration, etc. The notification be used to facilitate proper dispensing of the amount of the composition 106 and/or to facilitate proper application of force for nerve liberation. For example, a user can be instructed to depress the contact tip 112 until the notification is issued by the applicator 100. This can impede the user from only partially depressing the contact tip, which could result in an inadequate amount of dispensed composition 106 and/or inadequate force for nerve liberation.

A spring 122 or other biasing member can bias the contact tip 112 towards the neutral position. As the contact tip 112 moves closer to the fully depressed position, the amount of force that the spring 122 or other biasing member imparts onto the contact tip 112 can increase. The applicator 100 can be configured so that the signal is generated when a threshold amount of force is applied to the contact tip 112. The spring 122 or other biasing member can be selected so that the threshold amount of force is sufficient to facilitate nerve liberation. The signal can be used to trigger a notification to the user to indicate that the user has applied sufficient force to satisfy the threshold, or to provide an instruction to the user to press harder. Generally, enough force to visibly deform the superficial tissue (e.g., skin) is sufficient. If the user can see the skin being depressed, pinched, lifted, then that can be beneficial for liberating nerves. Depending on the particular nerve, in some cases more force may be needed.

In some embodiments, the applicator 100 can be configured to generate signals based on various amounts of force applied to the contact tip and/or based on various position of the contact tip 112. For example, the embodiments illustrated in FIGS. 6 and 7 can include multiple electrical contacts that are positioned at multiple depths along the path of the contact tip 112, so that the first electrical contact 138 can contact the multiple electrical contacts in series as the contact tip 112 moves between the neutral position and the depressed position. Signals can be generated based on which of the multiple electrical contacts are in contact, and those signals can be indicative of the position of the contact tip 112 and/or the amount of force applied to the contact tip 112 (e.g., how hard the user is pressing). Many variations are possible. In some embodiments, a proximity sensor can be used, which based on capacitance sensing, inductive sensing, magnetism, an optical sensor, or any other suitable proximity sensing technique. The proximity sensor can generate signals based on the position of the contact tip 112 relative to another location on the applicator (e.g., at the location where the second electrical contact 140 is shown). In some embodiments, a pressure sensor can be coupled to the contact tip 112 (e.g., at the location where the first electrical contact 138 is shown), and/or a pressure sensor can be coupled to the housing or dispenser or other location (e.g., at the location where the second electrical contact 140 is shown). The pressure sensor can provide signals based on whether the contact tip 112 is depressed sufficiently to contact the pressure sensor to a corresponding structure (e.g., at the location where the other electrical contact 138 or 140 is shown). The pressure sensor can provide signals based on how much pressure is applied to the pressure sensor by the corresponding structure, which can be an indication of how hard the contact tip is being pressed. The signals generated can be used by the controller 124 to perform actions, as discussed herein, such as to start a timer, to start a therapy feature (e.g., electrification, vibration, heating, or cooling), and/or to provide information to a user. The controller 124 can provide a notification (e.g., via the notification element 136) when the force applied to the contact tip 112 is above a first threshold amount of force (which can be an indication that sufficient force is applied to facilitate nerve liberation). The controller 124 can provide a notification when the force applied to the contact tip 112 is above a second threshold (e.g., which can be higher than the first threshold). That notification can be an indication that the user is pressing too hard. In some embodiments, the applicator 100 does not have a notification feature for too much pressure.

With reference again to FIGS. 6 and 7, in some embodiments, the contact between the first electrical contact 138 and the second electrical contact 140 can pass through a signal, (e.g., rather than to generate a signal that is delivered to the controller 124 in some cases). For example, the second electrical contact 140 can be coupled to the power source 126. When the first electrical contact 138 and the second electrical contact 140 are separated (e.g., FIG. 6), the electrical power is not passed through to the contact tip 112. When the first electrical contact 138 and the second electrical contact 140 are in contact (e.g., FIG. 7), the electricity from the power source 126 is passed through to the contact tip 112. That electricity can electrify the contact tip 112 (e.g., for electrostimulation), can activate a vibrator coupled to the contact tip 112 (e.g., for vibratory stimulation), can activate a heating and/or cooling element coupled to the contact tip 112 (e.g., for caloric or cooling stimulation). The initiation of the electrostimulation, vibration, heating, or cooling can serve as a notification to the user that sufficient pressure is applied and/or that the amount of the composition 106 is fully dispensed. In some cases, the controller 124 can be omitted.

The applicator 100 can be used to combine the manual liberation of the nerve, the therapeutic topical treatment of the composition, and/or one or more of the additional therapy aspects discussed herein (e.g., electrostimulation, vibratory stimulation, caloric and/or cooling therapy), which can be synergistic.

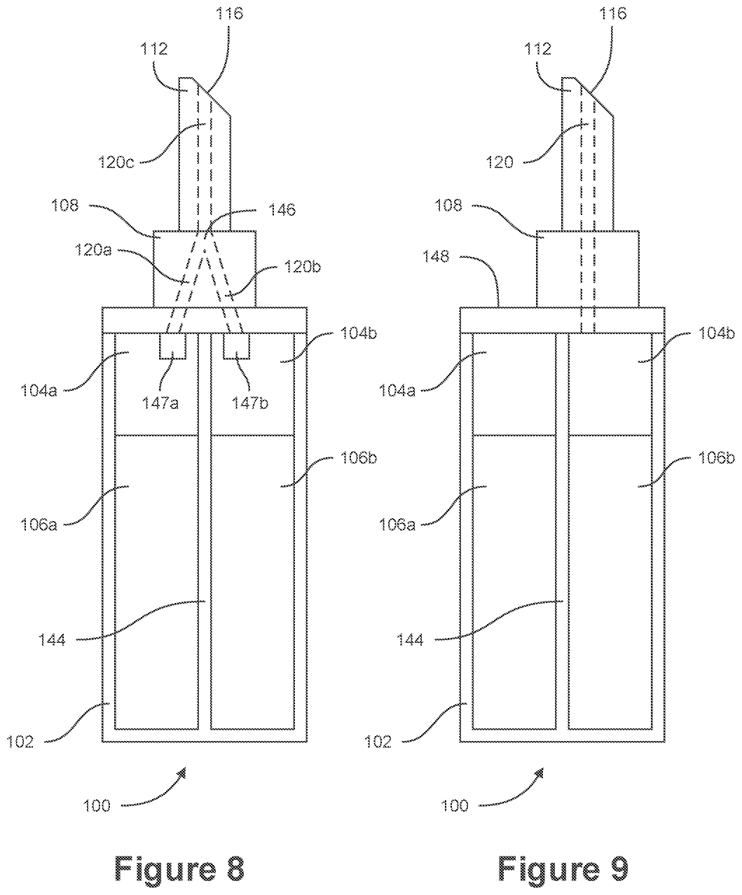

With reference to FIGS. 8 and 9, the applicator 100 can include multiple compartments, which can hold multiple isolated compositions. The applicator 100 can mix the compositions when dispensing, or the applicator can be used to select between dispensing the compositions individually. This configuration can be used to keep components of the therapeutic composition isolated from each other until close to the therapy time, such as until dispensing of the therapeutic composition. In some cases, ingredients of the composition can be reactive with each other, and/or the composition can be more stable when components are isolated. In some embodiments, one compartment can include a therapeutic composition, and another compartment can include a penetration enhancer, such as those discussed herein.

With reference to FIG. 8, the applicator 100 can have a housing 102 that defines an internal cavity that is divided into multiple compartments, such as a first compartment 104a and a second compartment 104b. Although two compartments 104a and 104b are shown, any suitable number of isolated compartments can be used (e.g., 2, 3, 4, 5, or more compartments). The contact tip 112 can have an opening 116, which can be in fluid communication with a plurality of the compartments 104a and 104b (e.g., with each of the multiple compartments) simultaneously, so that the corresponding contents 106a and 106b of the compartments 104a and 104b can mix upon when dispensed through the opening 116. In some embodiments, the therapeutic composition is not fully formulated until the corresponding components of the multiple compartments 104a and 104b are mixed. In some cases, the mixing of the components can be facilitated by the user rubbing the components against the skin (e.g., using the contact tip 112). In some cases, the first compartment 104a can include a first subset of one or more of the ingredients for the therapeutic composition, and the second compartment 104b can include a second subset of one or more of the ingredients for the therapeutic composition. In some embodiments, the first compartment 104a can include the therapeutic composition, and the second compartment 104b can include additional material, such as an additional therapeutic composition. A primary therapeutic composition (e.g., containing sugar, sugar alcohol, an/or an alkalizing agent) can be contained in the first compartment 104a, and a secondary therapeutic composition (e.g., menthol, camphor, phenols, polyphenols, salicylates, lidocaine, thymosin, pterostilbene and/or other analgesics disclosed herein) can be contained in the second compartment 104b. An internal wall 144 can separate the first compartment 104a and the second compartment 104b. Additional walls can be included (e.g., to isolate additional compartments).

A first conduit 120a can connect to the first compartment 104a, and a second conduit 120b can connect to the second compartment 104b. The first conduit 120a and the second conduit 120b can connect at a junction 146. In some embodiments, a third conduit 120c can extend from the junction 146 to the opening 116. In some embodiments, the junction 146 and/or the third conduit 120c can have mixing features, such as protrusion, bends, barriers, and/or constructions, that can encourage mixing of the contents 106a and 106b from the multiple compartments 104a and 104b. In some embodiments, the junction 146 can be at the opening 116, so that the contents 106a and 106b of the compartments 104a and 104b do not mix substantially until they are driven out of the opening 116. In some embodiments, the applicator 100 can include multiple openings, similar to the opening 116, so that the different contents 106a and 106b can be dispensed separately (e.g., but simultaneously). In some cases, the conduits 120a, 120b, and 120c can have different diameters or sizes, such as to control the amounts or proportions of the contents 106a and 106b that are dispensed from the compartments 104a and 104b. The compartments 104a and 104b can have different sizes, depending on the proportions or amounts of the contents 106a and 106b that are dispensed from the compartments 104a and 104b. For example, the first compartment 104a can have a larger volume than the second compartment 104b. The first conduit 120a can have a larger diameter or otherwise larger size than the second conduit 120b. In some embodiments, the applicator 100 can have one or more one-way valves 147a and 147b to impede mixing of the contents of the different compartments 104a and 104b. The one-way valves 147a can permit fluid to exit the compartment 104a, and can impede material from entering compartment 104a. The one-way valves 147b can permit fluid to exit the compartment 104b, and can impede material from entering compartment 104b. The one-way valves can be in the corresponding compartment 104a or 104b or in the corresponding conduit 102a or 102b.

With reference to FIG. 9, in some embodiments, the applicator 100 can be configured to change between separate configurations for dispensing the separate contents 106a and 106b of the separate compartments 104a and 104b. Although two compartments are shown in FIG. 9, any suitable number of compartments can be used. The applicator 100 can selectively dispense multiple compositions or ingredients. The contact tip 112 can be mounted onto a moveable structure 148, such as a rotating disc or portion of the housing 102. In FIG. 9, the contact tip 112 is shown at a position such that the conduit 120 is in fluid communication with the second compartment 104b (e.g., a second position). Movement of the moveable structure 148 can position the contact tip 112 so that the conduit 120 is in fluid communication with the first compartment 104a (e.g., a first position). The user can move the movable structure 128, such as by twisting an upper portion of the housing 102 relative to a lower portion of the housing 102, or by twisting a rotating disk relative to the housing 102, to move the contact tip 112 between the first position and the second position. In some cases, the contact tip can remain in one position while the applicator changes between the first configuration for dispensing the contents 106a of the first compartment 104a and the second configuration for dispensing the contents 106b of the second compartment 104b. For example, the conduit 120 can be moved between the first compartment 104a and the second compartment 104b, such as using the movable structure 148 (e.g., by rotating a disc or housing portion, or by toggling switch, or moving dial or other user input element). In some embodiments, the dispenser 108 (e.g., pump) can move relative to the housing 102 to change the position of the conduit 120 and/or contact tip 112. Additional compartments, and contents thereof, and corresponding applicator configurations can be used. Although not shown in FIGS. 8 and 9, the applicator 100 of those embodiments can also include the features relating to electrostimulation, vibratory stimulation, caloric stimulation, etc.

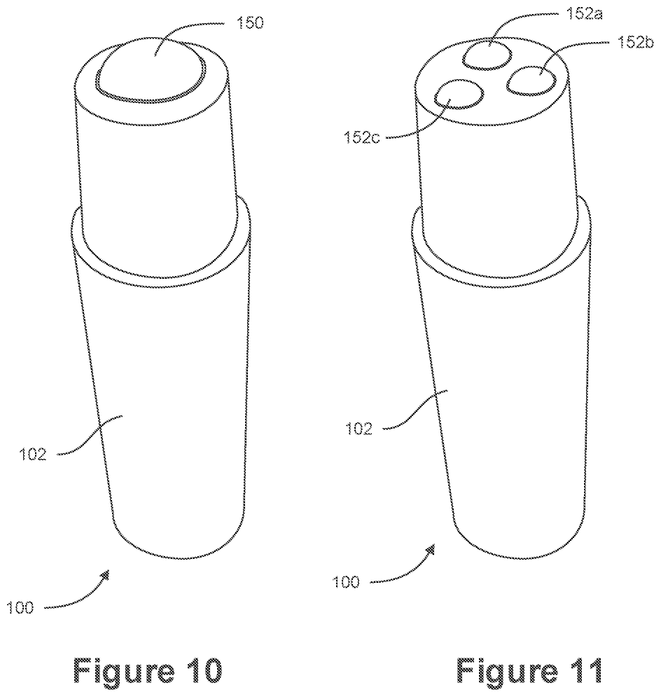

In some embodiments, the applicator 100 can include a roller ball dispenser 150 (e.g., as shown in FIG. 10), or a dispenser with multiple roller balls 152a-c (e.g., as shown in FIG. 11). In some cases, different roller ball dispensers 152a-c can be coupled to different compartments of the applicator 100, so that the different roller ball dispensers 152a-c can dispense different substances. Or multiple roller ball dispensers 152a-c can dispense the same substance, such as the therapeutic compositions disclosed herein. A roller ball dispenser can combine substances from multiple compartments (e.g., similar to FIG. 8) or can selectively dispense a selected substance (e.g., similar to FIG. 9). The therapeutic composition can include electrical transmission-enhancing agents (e.g. zinc or zinc oxide), which can also have analgesic benefits.

The applicator may include a mobile roller ball housing, that can move relative to a formed base. When the roller ball housing is depressed relative to the base, the additional therapy energy (e.g., electrical, vibratory, heating, cooling, etc.) can be activated, similar to FIGS. 6 and 7.

In some cases, one or more textured roller balls can be used, which can facilitate grip on the tissue. The one or more roller balls can include micro-needles or nano-needles, or texture that can create channels in the skin for the therapeutic agent, or otherwise facilitate transdermal delivery. In other embodiments, the one or more roller balls can be smooth or un-textured. The spatula-tip, or other contact tips, can also be textured or un-textured, and can include micro-needles or nano-needles to facilitate delivery. The texture can include ribs, ridges, studs, points, etc. or combinations thereof. The texture can anchor the tissue under the contact tip. The texture, micro-needles, or nano-needles can also transfer electrical current, heat, cooling, or vibration.

The applicator 100 can include a split septum dispenser or squeeze tip, in some embodiments. The housing 102 can have a deformable body, which the user can squeeze to dispense the contents. In some cases, the applicator 100 can be openable and reclosed, such as using a screw cap, so that the applicator can be filled or refilled. In some cases, the applicator can be sealed, such as for a single-use device that is not designed to be refilled. In some embodiments, the contact tip can include a suction cup. In some embodiments, the contact tip can include a suction cup, with a roller ball inside the suction cup (e.g., at the center), so that the user can press down to apply positive pressure via the roller ball, and can pull away to apply negative pressure via the suction cup.

In some embodiments, the applicator outputting an electrical current can be used to identify superficial sensory nerves, such as to identify locations to focus treatment. The user can move the applicator around the tissue while it outputs a mild electrical current, and when the mild current results in a comparatively high level of tenderness or pain, that can be an indication of a superficial sensory nerve. The user can then focus treatment, as described herein, on that identified superficial sensory nerve. In some cases, a different electrical signal (e.g., more current) can be applied during the treatment phase, as compared to the nerve-seeking phase.

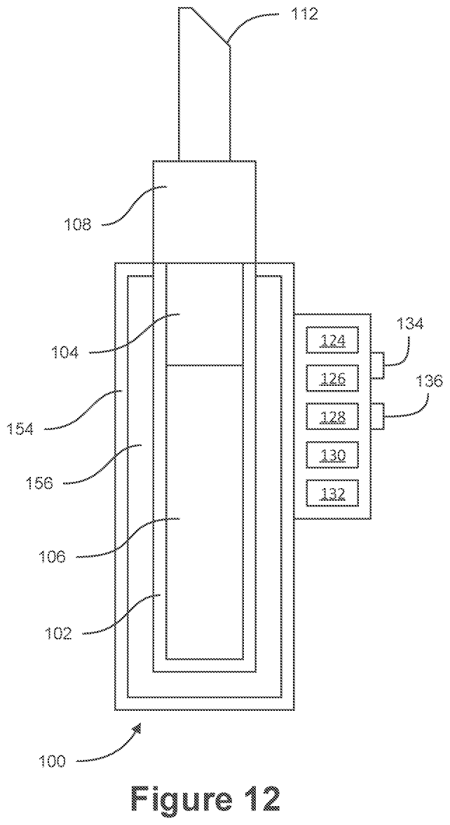

With reference to FIG. 12, the applicator can have an outer shell 154 that defines an outer compartment 156 that at least partially surrounds one or more inner compartments 104. The inner compartment 104 can have the therapeutic composition or other contents for dispensing. The outer compartment 156 can be isolated from the inner compartment 104. The outer compartment 156 can contain a fluid, gel, or other material for cooling or warming the inner compartment 104, such as freezer ice packs or warming pads. This embodiment can be beneficial to maintain or adjust temperature of contents, such as for travel or storage. This embodiment can be particularly useful for shipping live agents or agents particularly prone to temperature damage (e.g., including PRP, peptides and anti-CGRP (calcitonin gene-related peptide) agents). In some embodiments, the outer compartment 156 can include a thermally insulating material, or a vacuum.

In some embodiments, the applicator 100 can have a fluid accelerator mechanism. For high-pressure injection (e.g., without needles) of the therapeutic composition into the target tissue. This can be a single pulse or multiple, pulsatile pulses. The injection can be automated or actuated by user (e.g., via a button or other user input element). The high pressure injection can be effected by gas compression or a spring loading mechanism wherein a spring or deformable material is compressed (e.g., in a loaded position) and decompressed when a piston is freed from the loaded position. It can also take the form of a simple, assisted mechanism wherein a spring or similar mechanism is adding velocity to the human-actuated pump to accelerate the release of fluid medium (e.g., to overcome resistance of tissue).

The applicator embodiments can be used to treat various ailments described herein, such as relating to neurogenic pain. In some embodiments, a method of treatment can include identifying the target nerves, such as by correlation with symptomatic pattern and (where applicable) mechanism of injury and/or symptom provocation. Optionally, the skin can be prepared by cleaning and/or soaking in water or other preparatory fluid. The applicator can be positioned over a target nerve site, and physical force can be applied, such as positive pressure or negative (e.g., suction) pressure over nerve area. The user can make small linear or circular movements with the applicator while maintaining pressure. One can note a sharp spike in pain, which can resolve (e.g., 20-120 secs after application) as the nerve is liberated. Therapeutic energy cab be delivered via the applicator, which can be electrical, thermal, mechanical (e.g. friction, percussion, vibration, oscillation, excitation of underlying tissue by other force (e.g. sound and light, including laser light). The applicator can be used to release a therapeutic agent into target area. The treatment steps can be repeated (e.g., until the nerve is no longer sensitive at site or until a prescribed time limit has expired.) The user can then search for any other sore spots in the known distribution of the nerve, which can represent inflamed branches of target nerve. Those identified sites can be treated using the applicator, as described. In some cases, the user can treat the nerve path. In instances where the target nerve(s) is known to course generally parallel to the plane of the skin, the user can deliver therapeutic agent and therapeutic mechanical forces and/or energy (e.g., in any combination) along the known nerve pathway (e.g., even in the absence of confirmatory tenderness or sensory response. Palpatory pain, nerve percussion sign, numbness, burning, tingling, and the like can optionally be used to identify treatment sites. At times, manual percussion, palpation or directed exploration (e.g., based on the examiners knowledge of local nerve anatomy), can be directed to locate the tip of a nerve growth cone (the advancing tip of a previously-damaged, growing neural fiber). These tips are typically very sensitive and easily provoked.

For post-injury or post-surgical scar sites (e.g., associated with post-C-section, or total knee arthroplasty), the user can scan treatment (e.g., vertically and/or horizontally) to cover peri-scar (area of body tissue surrounding a scar, which can be exquisitely tender in areas that are difficult for patient to describe). These areas can be confirmed by pressing the applicator, or other device, in probing manner around this area then delivering agent and technique to symptomatic points, which can be where nerve branches are being ensnared. The treatment can also be applied to intra-scar tissue (e.g., inside the visibly or tactically-confirmed scar area where nerves can become ensnared.)

A method for treating headache (e.g., applied in acute or prophylactic approaches) can use the applicator, as described, on superficial nerves of face, and can be beneficial for treating headache disorders (e.g., including tension headache, migraine, and cluster headache). The target nerves can include supraorbital, supratrochlear, infraorbital, superficial temporal, greater- and lesser-occipital nerves, Erb's point, or any combinations thereof. This approach can be beneficial over therapy that stimulates branches of the trigeminal nerve (e.g., mesencephalic branches of the trigeminal nerve) only. The applicator treatment can be applied to provide stimulation of trigeminal, vagal, facial and other somatic nerves outside the cranial centers (e.g., the greater- and lesser-occipital nerves, which are frequently symptomatic in headache syndromes.) The headache treatment can use the applicator to provide electrical stimulation, thermal stimulation, and chemical treatment, as well as mechanical liberation of nerves, in some embodiments.

A method for treating neck pain can use the applicator, as described herein, to treat the following nerves or sites: Erb's Point, transcervical, supraclavicular, cervical segmental branches, greater- and lesser occipital, or any combination thereof. A method for treating lower back pain can use the applicator, as described herein, to treat the following nerves or sites: superior-, middle-, and inferior cluneal nerves; iliohypogastric; subcostal nerves, or any combinations thereof. A method for treating foot pain conditions can use the applicator, as described herein, to treat the following nerves or sites: sural; posterior tibial; medial- and lateral plantar; digital branches, calcaneal branch, superficial peroneal, or any combinations thereof. A method for treating knee pain can use the applicator, as described herein, to treat the following nerves or sites: femoral, saphenous, lateral femoral cutaneous, peroneal, or any combinations thereof. For example, the applicator can be used to provide electrical stimulation, thermal stimulation, and chemical treatment, as well as mechanical liberation of nerves, in some embodiments. The applicator can be used to provide electrical stimulation, chemical treatment, and mechanical liberation of nerves, in some embodiments.

The therapeutic method can proceed as follows: 1) A target nerve or group of nerves is identified; 2) a topical agent is applied to the target nerve area OR a mechanical force (e.g., positive pressure friction or negative (vacuum) pressure mobilization) is applied to mechanically liberate the nerve; 3) (optionally) An electrical current (or other physical energy) is applied to the target nerve/s and body tissues: 4) (optionally) an amount of topical therapeutic agent is reapplied to the target nerve/s.

The applicator can be used to treat peripheral neuropathy and peripheral nerve compressive neuropathies (e.g., carpal tunnel syndrome and diabetic peripheral neuropathy), median neuropathy at the wrist, ulnar neuropathy at the wrist, ulnar neuropathy at the elbow, and superficial radial neuropathy at the wrist. Also, the applicator can be used to treat neuropathic pain.

In some embodiments, the electrical current in tip of applicator can assist in driving the therapeutic agent beyond the protective skin layer to deeper tissues, and ultimately into systemic circulation. Using the applicator to apply the therapeutic agent can be better than manual application because the therapeutic agent would not be absorbed into the hand, which can increase penetration of the agent. The contact tip can be non-absorptive. The applicator can be beneficial over fixed electrodes, because the applicator can be easily moved from treatment site to treatment site. The applicator can facilitate accurate distribution of the therapeutic agent (e.g., drug), such as over nerve pathways, as the applicator can be manipulated like a pen. The topical composition can be electrically conductive, as well at therapeutic, which can facilitate administration of the electrical therapy, such as be enabling the electrical therapy to be achieved using less current and improved patient comfort.

In some embodiments, nerves can be located by changes in impedance at the skin.

Pain Treatments

Modern understanding of pain and persistent disability is evolving to appreciate a broader range of factors in response to which, the field of physical medicine and rehabilitation is compelled to evolve and deliver a new range of therapies and treatment approaches. There is a growing appreciation that traumatic injury to the body can manifest in multiple dimensions. For example, a knee injury has the potential to affect structures of the joint (e.g. bone, cartilage and ligaments), nerves in the area (e.g. superficial sensory nerves, motor nerves to muscle and joint receptors), muscle (incl. contractile elements and tendon), the central nervous system (e.g. spinal cord, brainstem and brain), and even produce cognitive, behavioral and emotional sequelae. While in many cases, injuries can resolve through the natural course of healing, in some cases, a certain dimension of the injury complex can remain unhealed, which can impair complete rehabilitation. Thus, modern pain rehabilitation approaches are well advised to follow an integrative, multi-faceted approach that addresses all domains of the injury. One of the most underappreciated sources of persistent pain and disability is neurogenic inflammation, especially inflammation of superficial nerves.

Initial muscle- and body tissue injury may set off a cascade of events wherein the persistence of pain is not myogenic but rather neurogenic (e.g., not caused by injury to muscle but rather by irritation to adjacent nerves). For example, acidic interstitial pH, associated with tonic contractions, spasm, or the early inflammatory process itself, may sensitize peri-muscular neural structures (incl. free nerve endings). Furthermore, local low-threshold mechanically depolarized nociceptors may be mechanically provoked by increased pressure associated with regional elevations in edema and increased concentration of ATP (resulting from injury of nearby muscle). Findings such as these would suggest that for optimal pain management and rehabilitation of muscle injury, a larger range of ‘chemical’ factors affecting nerves should be considered and addressed beyond currently available anti-inflammatory therapies That said, it appears that injury to muscle is not its only means of provoking nociceptive processes in nerves.

The biochemical, metabolic by-products of exertion, overuse, fatigue and damage of muscles can create an adverse physiologic perineural environment that pro-nociceptive, inflammatory and potentially (via reflex loops) inhibitory of muscle activation and strength. One of the various chemical changes associated with this process is the accumulation of protons (hydrogen ions) Muscles associated with stabilization of bipedal posture are particularly prone to this issue, including but not limited to the gluteal complex, paraspnals, deep cervical muscles, plantar and dorsiflexors of the ankle, and rotators of the shoulder. Thus, a solution that would simultaneously address the durability of muscle whilst simultaneously counteracting local processes that promote neural sensitization and inflammation would be of significant clinical value in a wide range of common musculoskeletal complaints, including lower back pain, Achilles tendinopathy, etc.

Surgery itself is an underappreciated source of persistent pain and disability. Pain associated with dermal scars is a highly underappreciated cause of decreased quality of life. Dysfunctional scars can lead to movement restriction, loss of sensation, itching, and persistent pain. Patients with painful scars might visit dermatologists or plastic surgeons, however, these highly-trained specialists often have great difficulty managing this type of pain. If a mature scar is still painful, one possible cause could be from regenerating nerves trapped in fibrotic dermal scar tissue. Unfortunately, for many sufferers, the classic signs of nerve entrapment (like positive nerve percussion) are not present, making diagnosis and treatment very difficult, often leading to suspicion of some sort of technical issue with the surgical procedure.

The skin houses abundant nerve fibers distributed over all the skin layers. They can communicate with different cell populations in the different layer of the skin by releasing various types of neuropeptides. Certain neuropeptides, include substance P and CGRP are highly inflammatory and pain-promoting. This means that it is possible for a positive bidirectional feedback loop able to occur that augments the inflammatory response surrounding a scar wherein a compromised dermal layer can (chemically and/or mechanically) provoke nerves, which in turn release inflammatory peptides, which then deepen the inflammatory process.

Some recent perspectives on the nature of joint pain further support the potential role of neurogenic inflammation (nerve inflammation) in presentations previously regarded as exclusively orthopedic in nature. Several tissues of the joint appear to be richly innervated, including synovium, ligament and tendon insertions, subchondral bone, periosteum and even the meniscus. Without being bound by theory, this could explain how inflammation and sensitization of superficial nerves overlying the joint may converge on fibers serving deeper structures as they create the perception of“deep joint pain”. Over time in osteoarthritis, sensory fibers may be depleted (apparently damaged and “retracted”) from the inflammatory environment of the joint, however, terminal endings associated with pain-related peptides may actually increase in density. This might lead to a joint that “senses less” (proprioceptively) but “feels more” (painfully), which is prone to further damage through poor motor control with an augmented response to injury, associated with disability and persistent symptoms.

Over-the-counter topicals are a particularly beneficial choice for pain and symptomatic relief with a number of advantages over injections and orally administered analgesics the ability to direct the therapeutic agent directly onto the region of perceived symptoms with the benefit of lowering the burden and risk of toxicity on systemic metabolism. Another limitation of medications that are administered by injection or orally, which is significantly mitigated by topically administration, is that many therapeutic compounds are broken down (e.g. by enzymatic breakdown) rapidly when delivered into systemic circulation (e.g. certain Flavonoids). Delivery through the skin allows for therapeutic agents to arrive directly at the area of complaint before they have been broken down by systemic metabolism. Despite these benefits, a number of challenges remain in the field of topical therapies.

Topically-administered therapeutics can include analgesics and anti-inflammatories such as counter-irritants (incl. camphor, menthol, peppermint), Capsaicin, Salicylates (incl. Wintergreen, Willow bark, Trolamine Salicylate), local anesthetics (e.g. Lidocaine, Articaine, Bupivacaine, Dibucaine, Etidocaine, Levobupivacaine, Mepivacaine. Prilocaine), Ketamine, and anti-inflammatories (incl. Corticosteroids, Methylsulfonylmethane (MSM), and non-steroidal anti-inflammatories (NSAIDs)). While these agents each hold therapeutic potential as combinations and monotherapies, in the context of topical applications, they all share a number of challenges to their optimal efficacy. These include: 1) the challenge of attrition of the therapeutic agent as the application passes through the protective outer layers of the skin, which can significantly dissipate the amount of therapeutic agent reaching the target tissues; 2) the challenge of delivering the maximal therapeutic dose while keeping the overall volume of the therapeutic agent delivered to the skin to a minimum, as some agents may pose increased risk for undesired side effects on the skin and superficial tissues when delivered in relatively larger amounts; and 3) the challenge of delivering the therapeutic agent in a vehicle that is appealing in texture and performance as it is applied by the user.

While many approaches in physical rehabilitation and medicine are aimed at damaged tissues, very few are aimed at altering biomechanics to rapidly offload the damaged tissue, thereby giving it an opportunity to heal. It is believed that many patients who undergo orthopedic surgeries, such as spinal fusion, knee or hip replacement or interventional pain procedures for musculoskeletal damage or degeneration, still retain the same biomechanical abnormalities that drove the structural damage over time. In such cases, the retained abnormalities in movement can promote damage in other joints, irritate local and remote tissues (incl. nerves) and even cause wear and tear to surgical sites and hardware. It would therefore be of benefit to introduce a physical modality or therapeutic approach that simultaneously mitigates pain whilst positively altering biomechanics to offload injured tissues and mitigate persistent pain and disability.

Some embodiments described herein relate to systems and methods for treating pain and symptoms of neurogenic origin and other conditions involving the human nervous system. Some implementations of the treatment system, can include an interface with the skin that is configured to cover one or more nerve territories. This interface, in combination with certain topical compounds, can maximize the treatment effect of the topical compound. When one or more application sites are involved, the combination of these might create an effect on the central nervous system in addition to the effects at the site of application, which might modify sensory, motor, cognitive or behavioral function of the nervous system, especially brain, brainstem or spinal cord.

System and Method for Using Topical Agents for Therapeutically Stimulating Symptoms and Disorders of the Central Nervous System

Some embodiments described herein relate to systems and methods for the application of a therapeutic compound that can elicit stimulation of target nerves or alteration of neural membrane threshold in order to increase the probably of summation of a target nerve or group of nerves, thereby increasing overall frequency of firing over a time period. Examples can include certain elements or ions that alter osmotic pressure and/or electrical gradient across the neural membrane, for example, including but not limited to sodium, calcium, potassium, and magnesium.

In some instances, the therapeutic compound can produce an overall inhibitory effect on the nerves to decrease afferent signals and consequently therapeutically reduce activity of target centers of the central nervous system. An example of an agent that can achieve such an end would be Lidocame or Bupivicaine. Another example of an agent that could be utilized as an inhibitory agent can include an agent that produces cooling or reduces sensation on skin or activity level of mechanoreceptors. Some examples of agents that can achieve an opposing, excitatory effect would be a dextrose- (or other sugar), mannitol- (or other sugar alcohol), or TOR-modulating compound or polyphenol-based compound. Another form of excitatory agent can be an agent producing a chemical or physical reaction that produces warmth or a tactile sensation on the skin, which stimulates mechanoreceptors. That said, any agent known to affect receptor- or neural membrane excitability toward a state of increased probability of summation is considered appropriate as a excitatory agents for this purpose.

In certain therapeutic applications of this method, one interface or a series of formed interfaces carrying and delivering specific but intentionally varied compounds, can be arranged in a particular therapeutic montage so as to impart different effects to each target site but intended to work in combination so as to impart a particular effect on the nervous system, thereby producing an intended clinical effect. In one embodiment, one or more interface site with the skin, overlaying a target body or nerve area, could be carrying a therapeutic compound, which might be imparting an inhibitory effect on the target tissue, while another single site or series of multiple interface sites might be delivering compounds with an excitatory effect on target tissues A number of therapeutic approaches have been developed to stimulate the autonomic nervous system and as a rule, these therapies are directed at one part of the autonomic nervous system (e.g., either the sympathetic or parasympathetic nervous system). In consideration of the benefits and risks associated with this general approach, an approach is suggested whereby relatively less therapeutic agent might be required if an approach is adopted to dampen one, while ramping the opposing aspect of the system. Some of the benefits to this approach are a faster response from the nervous system as well as a relatively reduced amount of therapeutic agent required to exert an overall effect (which can be termed as ‘see-saw effect’).

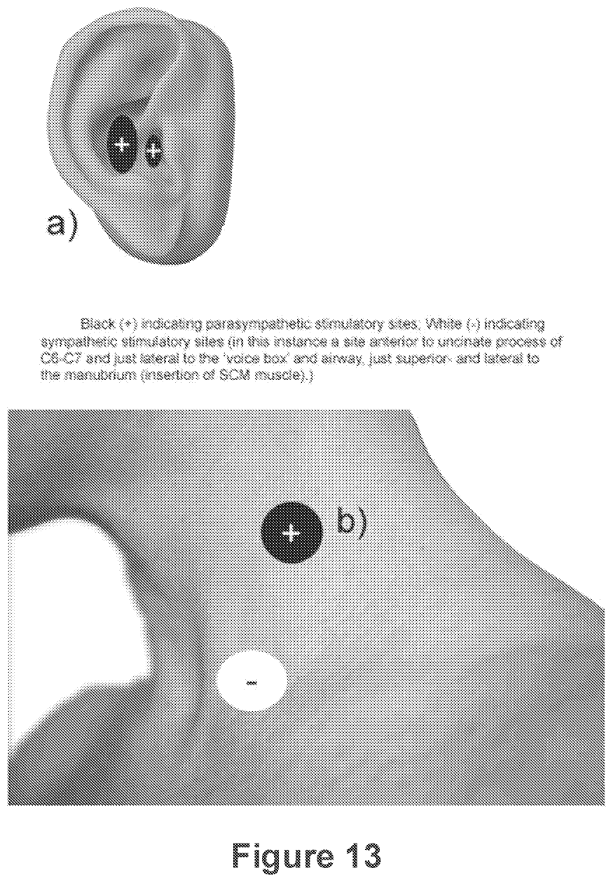

In one potential embodiment of the abovementioned strategy, it may be desired to affect the autonomic nervous system in such a way so as to relatively reduce sympathetic activity and relatively elevate parasympathetic activity. Toward this end, an excitatory compound can be delivered to the vagal- and/or glossopharyngeal nerves (appealing to the parasympathetic system), such as at the lateral surface of the neck (see FIG. 13b), whilst an inhibitory compound can be directed just above the stellate ganglion (appealing to the sympathetic nervous system), lower down on the neck (see FIG. 13b). Such a configuration can have an overall stimulatory effect on the parasympathetic nervous system with simultaneous inhibitory effect on the sympathetic nervous system. This can produce a variety of potential positive health outcomes, associated with improved status of the autonomic nervous system and its associated brain and body regions, such as improved sense of wellbeing, diminished anxiety or depression, reduced pain, improved digestive or eliminatory function, positive cardiovascular change, and improved sleep, reduced sweating of the hands, armpits and body, Raynaud's syndrome symptoms, mitigation of complex regional pain syndrome symptoms, among others. A formed interface (e.g., patch or bandage) can be shaped to fit the neck and cover sites for the vagal and/or glossopharyngeal nerves as well as the stellate ganglion. The area of the interface configured to cover the vagal and/or glossopharyngeal nerves can include the excitatory composition, and the area of the interface configured to cover the stellate ganglion can include the inhibitory composition. In another embodiment, directed to achieve a similar overall effect, which may be combined with the aforementioned application, a stimulating agent or compound can be applied to the concha of the ear (to access the vagus nerve for a parasympathetic effect) (see FIG. 13a), while an inhibitory agent or compound can be applied to another sympathetic nerve pathway or ganglion (see FIG. 13b). The benefit to this montage is the relatively more superficial availability of vagus nerve branches at the surface of the ear and ear canal.

While delivering the aforementioned agents on an interface can be advantageous as this can promote transmission across the skin into the target tissues and in the above described method, which can avoid risks associated with injection (primarily to vascular structures) by use of topical agents, it may be necessary (in some cases) or more practical at certain times to simply deliver these agents by another form of transmission through the skin for example by injection, iontophoresis, or topical application. It may also be indicated to enhance the intended function (e.g., excitation or inhibition of target structures) to deliver these agents in combination with another physical modality with an additive effect. For example, an excitatory- or inhibitory agent may be delivered in conjunction with a therapeutic laser, light source, mechano-stimulatory device, thermal source, sound source, electrical current source, or magnetic source, wherein this source of energy itself (or in combination with the therapeutic chemical compound) would have an inhibitory or excitatory effect on target tissues.

In yet another embodiment of the method, chemical stimulation (e.g. via application of a topical- or injectable stimulatory or inhibitory agent) at one or more sites, can be synergized with physical stimulation (e.g. by light (incl. laser light), mechanical stimulation, thermal stimulation (e.g., heating or cooling), sound stimulation, electrical- or magnetic stimulation, at another site or other sites.

In another embodiment of the method, therapeutic inhibition of the sympathetic nervous system can be enhanced by the simultaneous stimulation of superficial nerves and other nerves associated with the vagal, glossopharyngeal, facial, or other parasympathetic-associated branches. Some examples can include targeting the concha of the ear or canal of the ear (vagal); the carotid-sinus and/or -body at the lateral aspect of the neck, and intranasal fibers. In this application, while an inhibitory or suppressive agent and/or therapy is applied to the stellate ganglion region (SG) (or other sympathetic-associated nerve), an excitatory or stimulatory agent and/or therapy is applied to the parasympathetic-associated nerve or fiber (see FIG. 13). In an example embodiment of this method, the inhibitory/suppressive and excitatory/stimulatory agents and/or therapies would occur on the same side of the head, but these applications can also be executed on ipsilateral-, contralateral- or bilateral sides of the head or body. The abovementioned autonomic-associated montages might be supplemented by the simultaneous delivery of physical forces, such as vibration, oscillation, percussion, electrical-, magnetic-, light-, thermal-, pressure-, light- or sound energy, as described herein in a device system and method. Furthermore, with specific respect to electrical- and magnetic-energy, these physical forces might be delivered in a manner that results in inhibition or suppression of neural impulses in the sympathetic tissue (e.g. SG) and facilitation or excitation in the parasympathetic tissue. In some instances, it may be of value to reverse the orientation of excitatory vs inhibitory stimuli such that the sympathetic component is being relatively inhibited while the parasympathetic component is being relatively excited. In another example of a certain embodiment of this system and method, the peripheral nerve stimulatory agents and/or therapy applications may be supplemented with mental (e.g., cognitive or behavioral) tasks intended to either stimulate or inhibit the limbic system. For example, for a particular therapeutic application, the limbic system can be inhibited while at the same time the sympathetic inputs to the system are reduced and inhibited. Many variations are possible, as the limbic system can be either stimulated or inhibited while the sympathetic system is either inhibited or excited, and/or while the parasympathetic system is either inhibited or excited, as discussed herein.

Thus method and system can be utilized for any condition that is associated with elevated sympathetic activity, and/or imbalanced or dysregulated autonomic activity. Such conditions might include POTS (postural orthostatic tachycardia syndrome), behavioral or affective conditions (e.g. anxiety, depression, post-traumatic stress disorder, mood disorders, addiction, eating disorders, personality disorders, and other cognitive- or behavioral disorders), seizure, dysautonomia, and chronic pain.

Therapeutic Skin Interface System and Therapy Method for Treatment of Pain, Neurogenic Inflammation and Other Symptom-Producing Conditions

Another device and method pertains to the therapeutic delivery of synergistic energy to target symptomatic nerves of the body. Embodiments of this device can be applied in the same manner as the abovementioned method (wherein a single- or multiple nerves can be subject to a certain excitatory stimulus, while another one or more nerves might be subject to a relatively inhibitory stimulus). As previously mentioned, this approach can be particularly effective when addressing the autonomic nervous system What follows is a description of a device and therapeutic method wherein, as a general description, one or more superficial nerves can be therapeutically modulated by an intentional combination of physical- and chemical forces, such as to affect both peripheral- and central nervous system.

In one embodiment, a first component of the device system, a therapeutic skin or body tissue interface, can take the form of a malleable, body-curve-conforming adhesive tape. This interface can be constructed of rigid-, flexible- or elastic tape (e.g., similar to available athletic tape). While the resistance to stretch of any embodiment of this device might vary, the commonality between potential materials is that interface that this material forms with the skin should be suitable to maintain contact with the skin whilst the user moves in a fashion consistent with generally common activities of daily living for a typical person. The interface with the skin might be anchored, by a form of adhesive (e.g. sticky hydrogel) or surface that resists glide across the skin or accidental removal, or a more advanced anchoring system (e.g. microneedles, suction cups, or textured surface). The various interface embodiments disclosed herein can include various features disclosed in U.S. Patent Application Publication No. 2017/0360867, which is incorporated herein by reference.

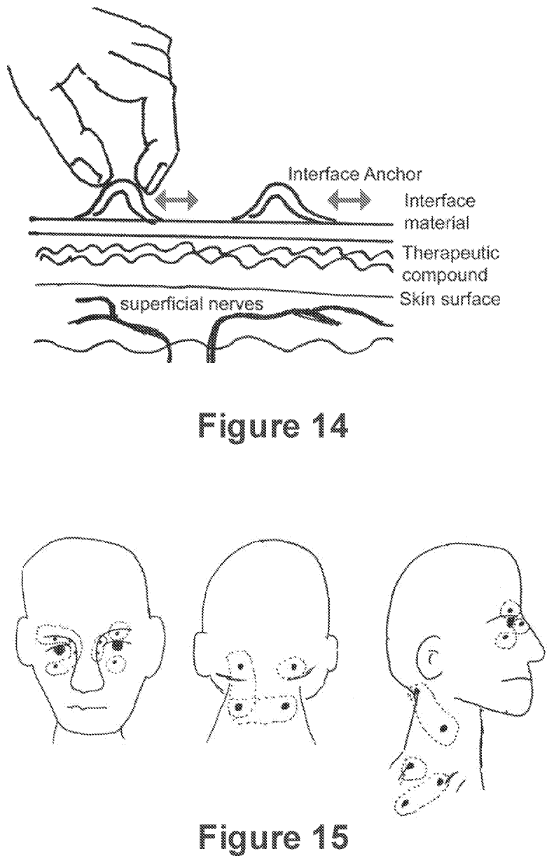

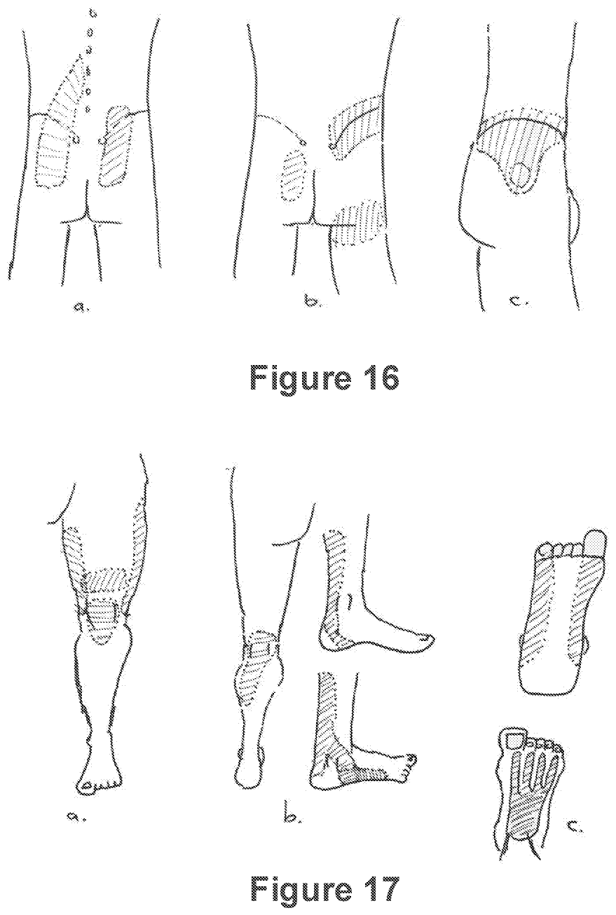

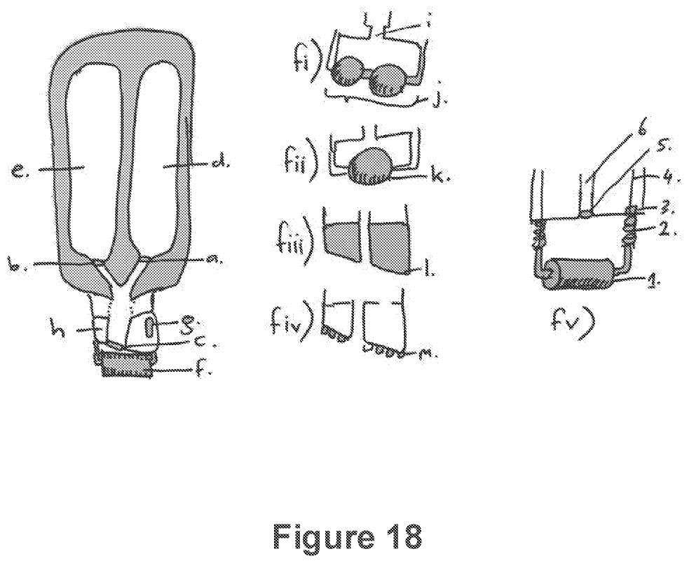

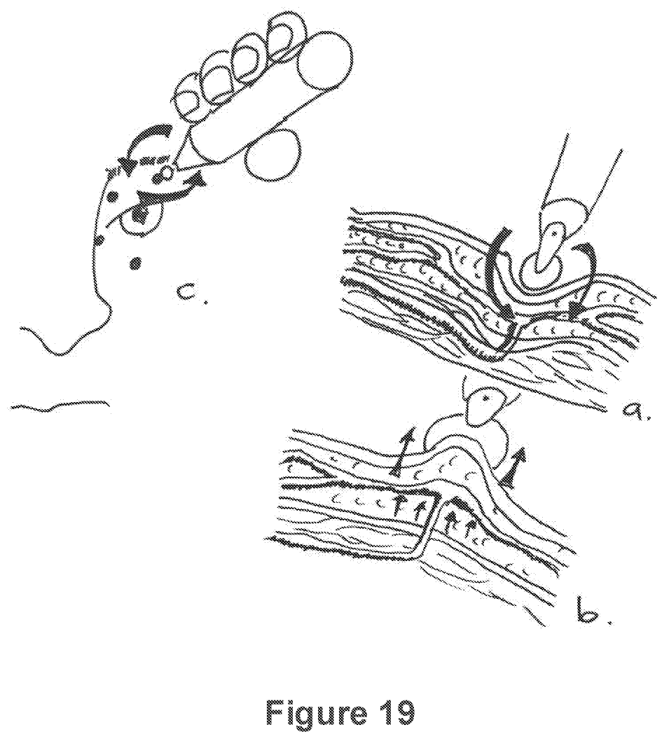

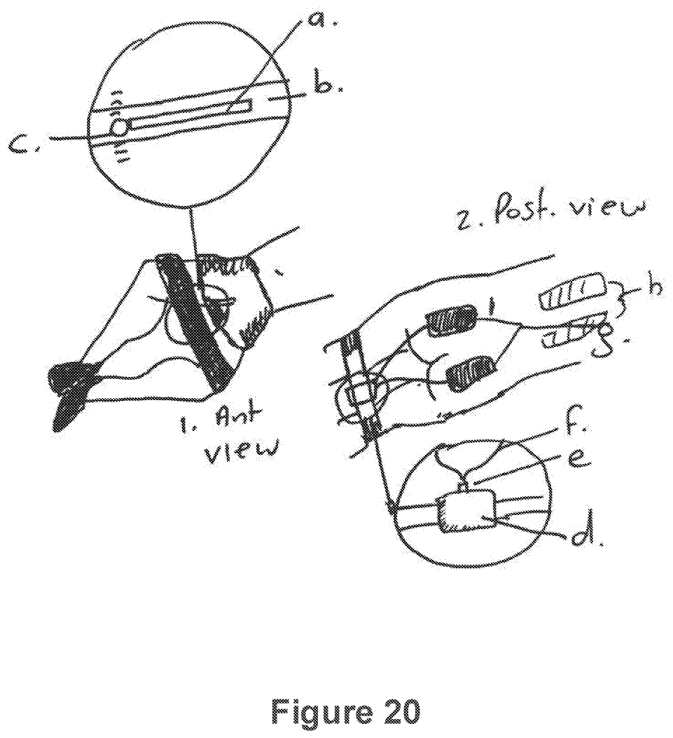

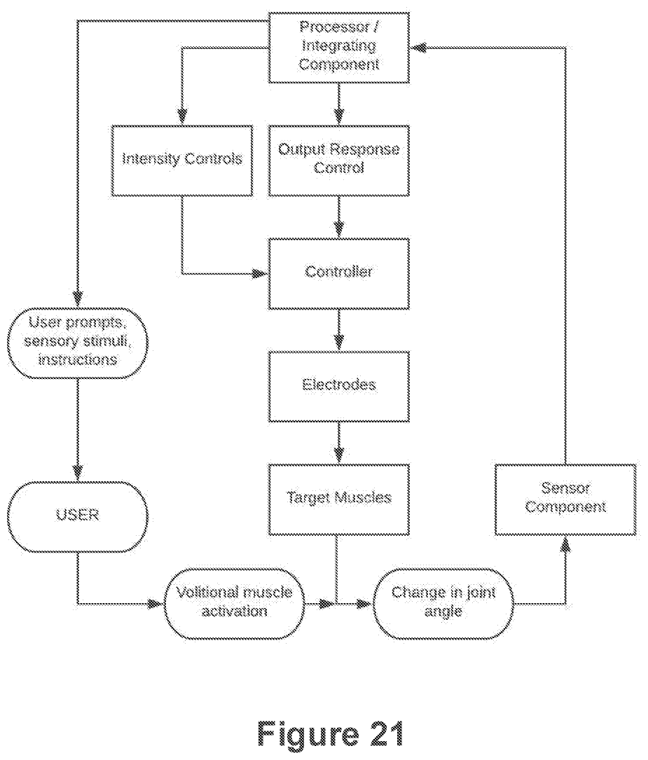

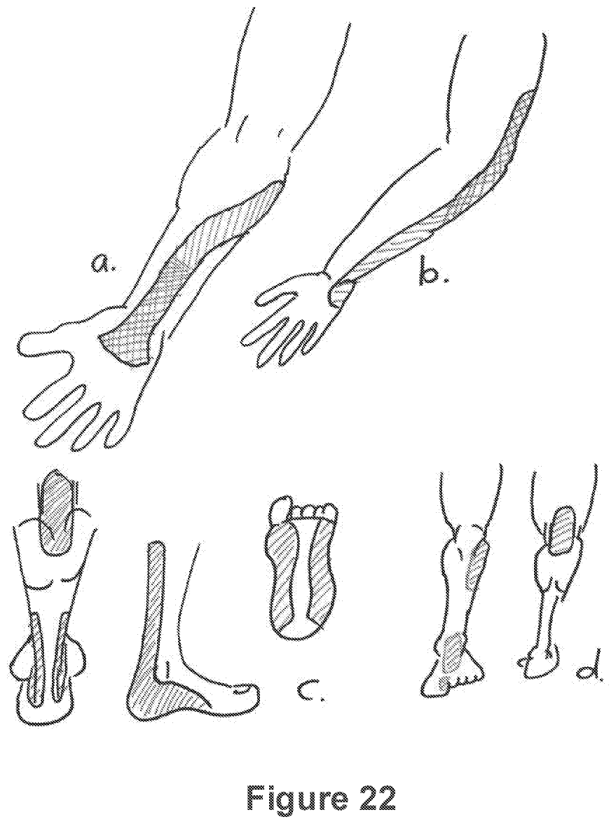

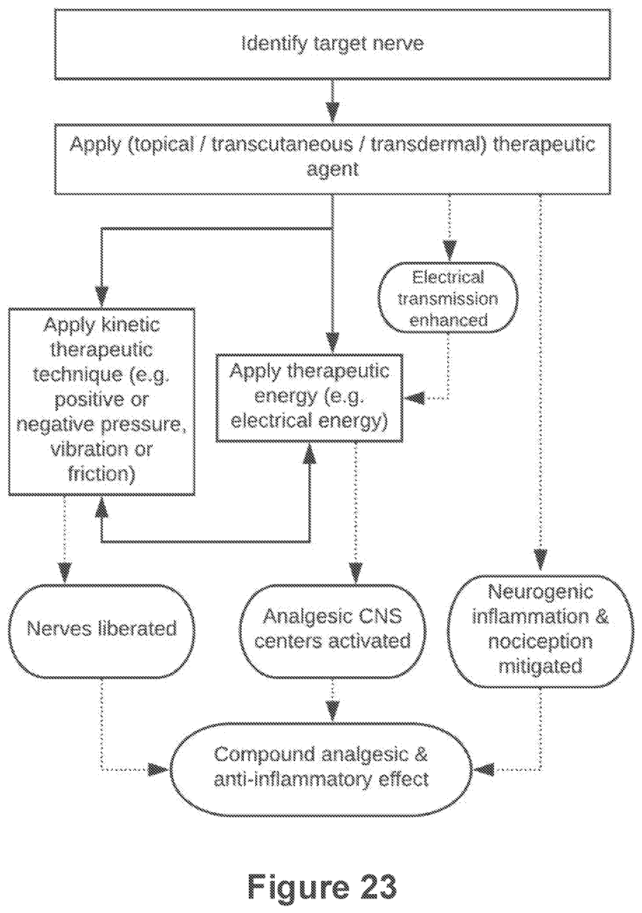

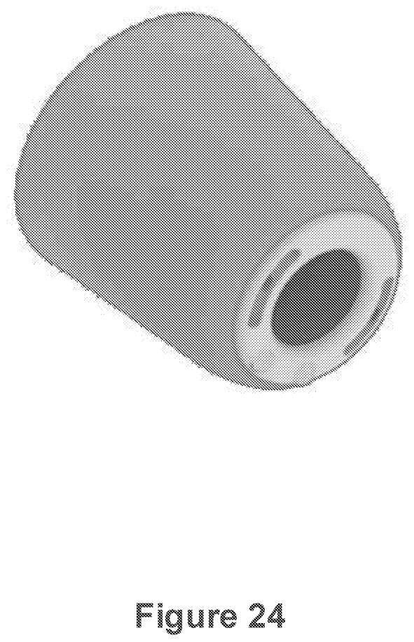

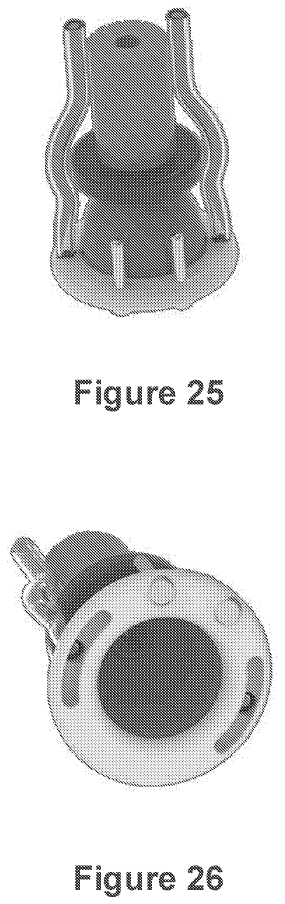

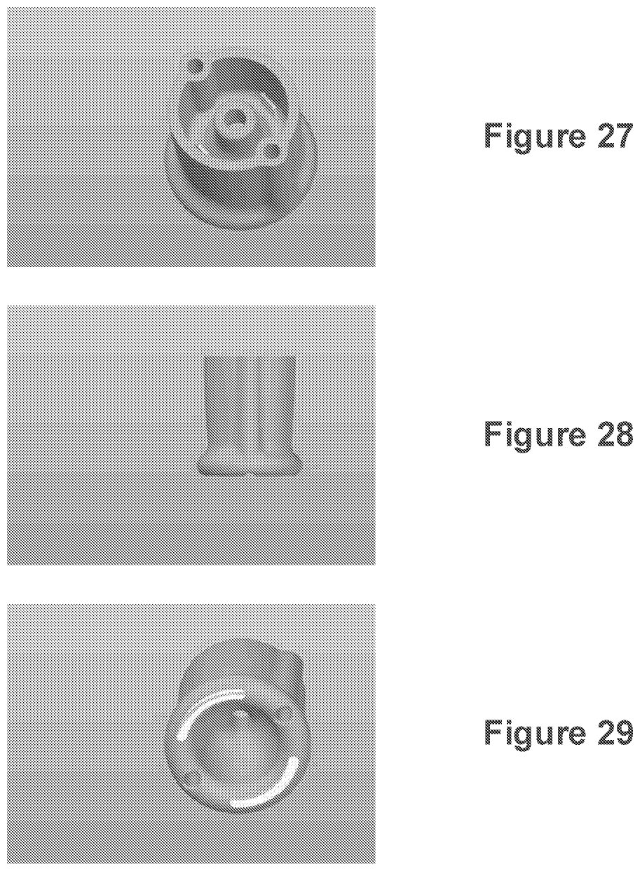

In an extension of this particular embodiment, following application to the skin surface, the interface can be physically or mechanically manipulated by a variety of sustained or variable forces to impart pressure, traction, oscillation, vibration or change in integrity of superficial and deeper tissues. This manipulation of the adhered interface might be enabled or facilitated by anchor points embedded or continuous with the interface material. For example, these might resemble handles, loops, or grips, which facilitate the hand or device to grab the interface (see FIG. 14). This is a particularly valuable intervention in situations where nerve structures or adjacent tissue might be imparting dysfunctional or pathologic restriction or mechanical irritation to one or more nerves or adjacent tissue, which could be producing undesirable symptoms. This method might also be enhanced chemically if the interface is prepared with therapeutic compounds, which are intended to impart certain physiologic effects on the target tissue to compliment the abovementioned, therapeutic mechanical forces. An example of this would be a compound containing any combination of: an amount of sugar (e.g. dextrose) and/or sugar alcohol (e.g. mannitol); a permeation enhancer (e.g. lecithin): a polyphenol (e.g. pterostilbene); a cannabinoid (e.g. Cannabitriol (CBT), Cannabinol (CBN), Cannabigerol (CBG)): an analgesic (e.g. menthol or camphor); an anesthetic (e.g. lidocaine). This said, the compound might be comprised of any topical compound with properties known to modulate neural function.

In some instances, a certain therapeutic approach, as a sequential step in a therapeutic process, could call for the intentional removal of the interface from the skin surface as in order to intentionally remove the outer protective skin layer in order to facilitate the subsequent transmission of therapeutic agent through the skin In the case of addressing pain and neurogenic inflammation, the initial strip of interface material may be placed over a known nerve pathway or region of multiple superficial nerves, such that with the removal of the adhering interface, the skin is rendered more porous and permeable over the pathway of the nerve, increasing the probability of successful delivery of a topical therapeutic agent to the target tissue and nerve.

In extension of the discussion regarding the immediately preceding concept, in a certain embodiment, a particular component (resembling a grip or handle) could be anchored to the interface material to facilitate subsequent manual or mechanical manipulation or movement of the interface (see FIG. 14). In this manner, the superficial tissues will passively follow any direction or movement of the adhered interface. The user could potentially apply a variety of physical forces, including traction (as the user pulls on the grip component), oscillation (as the user introducing fluctuating physical forces to the grip and/or interface material), compression (as two distant points are approximated), and/or stretching (as two points are moved apart). This approach overcomes a pitfall of friction, massaging or percussing devices or techniques of the skin that are unable to anchor to the superficial layers of skin in order impart sufficient physical force to achieve a therapeutic objective such as breaking adhesions or scar tissues, liberating nerves or manipulating tissue for any given therapeutic or cosmetic purpose. The interface could be applied as a tool or component utilized in the physical therapeutic approach to scar tissue and neurogenic inflammation described below. A simplified process representation of this process can be described in the following manner: Identify Target Nerve or Tissues→(optionally) Apply adhesive preparatory strip along path of target tissue/s or nerve/s→rapidly remove it to remove one or more superficial layer(s) of skin→apply therapeutic compound on interface (optionally with or without an interface)→manually (or with device) anchor to the interface→in cyclical or sustained fashion apply physical manipulation to the interface for period of time→(optionally) check for relief of symptoms→(optionally) repeat some or all of the process.

It should be noted that in most cases, while the use of mechanical (incl. manually-actuated) forces imparted to the skin interface can be valuable as part of the therapeutic process, in certain cases, the simple application of an interface carrying a therapeutic compound, directed to superficial tissues and nerves would be adequate and appropriate to achieve a therapeutic effect and indeed represent a and valuable treatment approach for a variety of body conditions.

In certain instances, when a topical therapeutic compound is utilized, it may include an amount of a suitable exfoliant or enzymatic exfoliant (e.g bacilus ferment or alpha hydroxy fruit acids), which can facilitate transmission of the primary therapeutic compounds transdermally by gently counteracting the superficial, protective layer of skin with the synergistic effect of enhancing skin appearance in a cosmetic manner—thereby exerting a dual, therapeutic and cosmetic action, which would be particularly desirable for application in conditions that might benefit from addressing the superficial nerves of the face, although this may also be true of other areas of the body. This effect might by synergistically supplemented or alternatively achieved with the addition of an amount of Niacinamide (nicotinamide) (e.g., ranging from about 0.1-15% total volume), which synergistically enhances the anti-inflammatory effect of the therapeutic topical, whilst repairing the skin surface.

With respect to the aforementioned purpose of providing an inhibitory effect on specific superficial nerves, it may be desirable to deliver this inhibitory effect in using a synergistic combination of an electrical field and a chemical agent. These forms of therapeutic treatment can be used individually, sequentially or simultaneously. An example embodiment of this concept is an extension of the aforementioned skin interface concept wherein a particular interface is intentionally placed on the skin over a known nerve pathway or anywhere along the nerve pathway between (the primary symptom-generating site; the site of perceived pain; or the distal point of the nerve pathway) and the spinal cord, brainstem or brain. In this particular application, the interface might be imbedded with a current-generating circuit or electrical system configured to deliver a certain, inhibitory electric- or electromagnetic field to the target nerves. An example current delivered by this system would be in the form of a negatively charged (monophasic) direct current optionally ranging from about 0.5 to about 3 milliamperes. While this system is by design intended to function in the lower current ranges for typical applications wherein inhibition of nociceptive- and autonomic fibers are targeted, for other applications, current may reach as high as about 5 amperes, thus the example embodiment of this device system current range is about 0.5 milliampere to about 5 ampere. This might be delivered in any form but one example would be direct current (DC) galvanic stimulation. The current can be dictated by the location on the body and considerations of safety (e.g., a relatively lower current can be used on the neck, in vicinity to autonomic nerves and relatively higher on the lower limb and buttock where there is less danger of visceral issues and much more tissue to penetrate). The power source for this application can be derived from a chemical reaction at the skin or by reaction with the skin and the chemical agent embedded on the interface surface. An alternative power source, especially for achieving higher currents, is a battery or an external power source that can be attached to the interface circuit, such as at a specific point.

With respect to an example embodiment of this application, a tape, bandage, patch, or other interface, which houses a particular electrical circuit might be designed to include regular (e.g., periodic) positive and negative terminals along its length such that the tape can be cut regular (e.g., periodic) intervals, such as at these nodes before applying to the target area of the body. At this point, a power source can be electrically coupled to the positive and negative nodes, now at the either or both ends of the circuit. Other embodiments may take the form of pre-formed, pre-cut tape, bandages, patches, or other interface types, which in some cases can be offered wherein the electrical components (e.g., a printed circuit) is coupled to terminals (e.g., at either end) either pre-attached to a power source or awaiting connection to an external power source or to a power source tethered or otherwise coupled to or incorporated into the interface at the time of application to the body. In yet another embodiment, a reaction between interface constituents and body tissues may produce the desired current upon contact (e.g., without the need for an external power source.)

Particular benefits of the abovementioned embodiment include: 1) analgesia is produced rapidly by the electric current, whilst the embedded chemical takes relatively longer to ramp in effect (e.g., the user enjoys a combination of rapid symptom relief as well as longer lasting benefits of the chemical agent); 2) this combined analgesic effect allows for more comfortable or even painless physical manipulation (including the technique described in this document) of otherwise uncomfortable nerves; 3) the electrical current can be used to enhance permeation or activation of therapeutic agents; 4) the medium for the therapeutic topical can contain aqueous electrocoupling agents to aid in transmission of therapeutic electrical signal. These effects are particularly true in cases of neurogenic inflammation and for this reason, this device and method can be particularly suited and intended for that purpose (while other indications are also possible). For this reason, as an example, a specific compound combination for this embodiment would include a therapeutic compound, including any combination of one or more (in any combination) of an amount of dextrose and/or mannitol, a buffering (e.g., alkalizing) agent, a polyphenol, a cannabinoid (e.g. Cannabitriol (CBT), Cannabinol (CBN), Cannabigerol (CBG)), an analgesic, an anti-inflammatory, or an anesthetic agent, or current-enhancing agent (e.g., an agent intended to enhance the transmission of electricity to and through the external skin membrane for example, a silver/silver chloride colloidal matrix or hydrogel.)

Returning to the concept of formed interfaces with the skin and the intention to shape these to target one or more superficial nerves of the body (see FIG. 15). One embodiment could be a condition-specific form to intentionally address certain superficial nerves (one or more), known to contribute to particular conditions or symptoms: One example would be a form intended to target varying combinations of one or more of the supraorbital, supratrochlear, infraorbital, nasal, erbs point, lesser occipital, greater occipital, tranverse cervical, posterior-, lateral-, and/or medial-suprascapular nerves, such as in order to treat headache disorders (e.g., migraine, cluster headache, tension headache, exertional headache, trigeminal neuralgia and pseudotumour cerebri) or conditions associated peripheral or central nervous system irritation (e.g., sinusitis, seizure (epilepsy), tinnitus). This particular embodiment can serve as a valuable alternative to devices that have effects on the trigeminal system such as transcutaneous electrical stimulation devices, in the sense that such an option would be hands-free, devoid of any uncomfortable electrical signals, and likely much cheaper and of lower risk as a therapy. Furthermore, this option could also provide a prolonged stimulus or effect (outlasting forms of stimulation that would otherwise be limited by for example, battery life and limits of reason in practicality of hand-held, user- or clinician-actuated treatments.) Yet another advantage is the ability to simultaneously address multiple nerves at once, over a prolonged period of time, not possible by current single-nerve or single-site stimulating options.

In one embodiment of the above, a single therapy application might contain multiple separate treatment sites imparted by multiple body interfaces, while another embodiment might appeal to multiple nerves or groups of nerves via a single continuous interface form. In one particular embodiment, the single formed interface can take various potential forms, such as a single linear strip wherein the strip is a continuous treatment surface delivering a therapeutic compound and/or therapeutic energy along its entire length: a form that is reduced and expanded at various points along its length so as to conveniently avoid certain body structures whilst covering certain target nerves (e.g., covering the supratrochlear and supraorbital nerve but avoiding the eyebrow or orbit of the eye; or, covering the distal sensory branches of the femoral nerve but avoiding the upper thigh and patella): a series of nodes connected by narrower internodes (similar to the aforementioned concept wherein the narrower internodes might carry some form of printed circuitry or therapeutic compound). In appealing to multiple sites or nerves, a single strip or other interface structure can appeal to each body target or nerve with a different compound or form of energy such that a single therapeutic application might appeal to numerous sites with multiple different energies or compounds (e.g., a particular site might be addressed with an inhibitory topical compound, whereas another might be stimulated with a combination of electrical energy and an excitatory topical compound in a synergistic manner, whereas yet another site in the same application might receive and inhibitory topical compound.)

While an approach of appealing to multiple target sites within as few strips as possible can be an advantageous embodiment in some situations, the device and method concept can be embodied in a therapeutic montage that includes multiple independent therapeutic sites. For example, this approach can be characterized wherein a single target nerve is served in a stand-alone manner by a single interface, which may include any number or combination of physical or chemical energy/treatment forms. Two example embodiments include firstly, a form of sugar and/or sugar alcohol (e.g., dextrose and/or mannitol) in a form of delivery vehicle wherein the delivery vehicle could be any combination of emollient, permeation enhancer, antimicrobial, emulsifier and humectant; and secondly a form of mild electrical current, which can be externally powered or powered by an interaction between the body and interface components or between components of the device interface. An example embodiment of this concept will release a mild current, which is direct, negative, monophasic and/or continuous but also may be any form of frequency, waveform, amplitude, intensity, etc. to achieve a particular therapeutic purpose.

Now to a number of potential embodiment configurations, where each represents a method for addressing particular conditions and symptoms In one configuration of the abovementioned embodiment, a therapeutic method for forms of headache and symptom disorders of the head (e.g. migraine, cluster headache, tension headache, sinus headache, trigeminal neuralgia) might be constructed where therapeutic interface is directed at multiple superficial nerves of the head and neck. This (or any aforementioned approaches to interface or montage configuration) might include simultaneous or sequential therapeutic treatment of any combination of the supratrochlear-, supraorbital-, lacrimal-, infraorbital-, infratrochlear-, nasal-, ophthalmic-, maxillary-, mandibular-, temporal-, mental-, buccal, auriculo-temporal, transverse cervical, lesser occipital-, greater occipital-, cervical ramus- (at one or more cervical levels), and greater auricular-nerves. Furthermore, this montage can be supplemented with all or a portion of the autonomic configuration illustrated in FIG. 13 wherein the sympathetic nervous system can consequently be inhibited, the benefit of which is to add a unique autonomic modulation opportunity to an approach that would otherwise only appeal to somatic fibers and principle sensory nuclei of the brainstem and spinal cord. The therapeutic interface in each nerve instance (or uniformly across each interface in a particular therapeutic application) can include a therapeutic topical compound, such as gel, cream, foam, liquid, clay, colloid, semi-solid, or malleable compound of any form that would incite a therapeutic response from target or underlying peripheral nerves. An example embodiment of a topical compound would include dextrose and/or mannitol (e.g., optionally in the range of 2-50% total compound volume or weight) with or without a buffering agent (e.g., that would shift pH to a relatively higher pH value) and a carrier vehicle. The topical compound could also include the aforementioned agents in conjunction with an amount of any one or combination of cannabinoid (e.g. Cannabitriol (CBT), Cannabinol (CBN), Cannabigerol (CBG)), analgesic (e.g. menthol), anti-inflammatory (e.g. corticosteroid), permeation enhancer (e.g. lecithin), polyphenol (e.g. pterostilbene), cannabinoid, anesthetic (e.g. lidocaine), regenerative agent (e.g. peptide, peptide fragment or PRP), and immune modulator (e.g. CGRP inhibitor). An example current emitted by an electrical interface would be direct, monophasic, negative and/or continuous in nature but can be of any form that would impart an effect on the target peripheral nerve or central nervous system target.

In one configuration of the abovementioned embodiment, a therapeutic montage for sinus and seasonal- or environmental allergy symptoms can be constructed. This (or any aforementioned approaches to interface or montage configuration) can include simultaneous or sequential therapeutic treatment of any combination of the supratrochlear-, supraorbital-, infraorbital-, infratrochlear-nerves. Furthermore, this montage can be supplemented with all or a portion of the autonomic configuration illustrated in FIG. 13 wherein the sympathetic nervous system can consequently be inhibited.

In another configuration of the abovementioned embodiment, a therapeutic montage for leg or knee pain (e.g. Osgood-schlatter's, chondromalacia patella, arthritis, fracture, or post-exertional pain) can be derived to address one or more in any combination of the saphenous-, femoral-, lateral femoral cutaneous-, infrapatellar-, femoral articular branches, (common, deep and/or superficial) peroneal-, cutaneous branch of obturator-, intermediate femoral cutaneous-, intermediate femoral cutaneous-, medial femoral cutaneous-, lateral cutaneous branch of the subcostal-, ilioinguinal-, genital-, infrapatellar branches, sural- and tibial-nerves. Furthermore, this montage can be supplemented with all or a portion of the autonomic configuration illustrated in FIG. 13 wherein the sympathetic nervous system can consequently be inhibited.

In one configuration of the abovementioned embodiment, a therapeutic montage for hip conditions (e.g. post-surgical hip pain, bursitis, fracture or post-exertional pain) can be constructed. This (or any aforementioned approaches to interface or montage configuration) can include subcostal-, iliohypogastric-, superior cluneal-, middle-cluneal-, inferior cluneal-, posterior femoral cutaneous-, lateral femoral cutaneous-, femoral branches, and lateral femoral cutaneous-nerves. Furthermore, this montage can be supplemented with all or a portion of the autonomic configuration illustrated in FIG. 13 wherein the sympathetic nervous system can consequently be inhibited.

In one configuration of the abovementioned embodiment, a therapeutic montage for lower back conditions (e.g. pre- or post-surgical lower back pain, mechanical lower back pain, or post-exertional pain) can be constructed. This (or any aforementioned approaches to interface or montage configuration) can include subcostal-, iliohypogastric-, superior cluneal-, middle-cluneal-, inferior cluneal-, posterior femoral cutaneous-, lateral femoral cutaneous-, femoral branches, and lateral femoral cutaneous-nerves. Furthermore, this montage can be supplemented with all or a portion of the autonomic configuration illustrated in FIG. 13 wherein the sympathetic nervous system can consequently be inhibited.

In one configuration of the abovementioned embodiment, a therapeutic montage for foot and/or ankle conditions (e.g. pre- or post-surgical for or ankle pain, Achilles tendon pain, plantar fasciitis, ankle sprain- or strain, superficial peronail neuropathy, metatarsalgia, morton's neuroma, tarsal tunnel, sural neuropathy, post- or intra-exertional syndromes) can be constructed. This (or any aforementioned approaches to interface or montage configuration) can include plantar-nerves, posterior tibial nerve, interdigital saphenous nerve, peroneal nerve, interdigital branches. Furthermore, this montage can be supplemented with all or a portion of the autonomic configuration illustrated in FIG. 13 wherein the sympathetic nervous system can consequently be inhibited.

In one configuration of the abovementioned embodiment, a therapeutic montage representing a treatment approach to specific, nerve conditions (neuropathies) can be constructed. Such a configuration can enable a targeted or prolonged therapy over a single nerve or multiple nerves involved in producing symptoms or signs. These potential nerve syndromes can include classic neuropathies such as carpal tunnel syndrome, ulnar neuropathy at the wrist or elbow, radial tunnel syndrome, superficial radial neuropathy (cheiralgia paresthetica), suboccipital neuritis, common peroneal neuropathy at the knee, superficial peroneal neuropathy at the ankle, tarsal tunnel syndrome (posterior tibial neuropathy), plantar neuropathy, peripheral polyneuropathy (including diabetic-, toxic-, metabolic-, hereditary-, acquired- or autoimmune-neuropathy). While the aforementioned classic neuropathies are example applications for this embodiment, they do not represent a complete list as some nerves of the body can be involved in unique patterns or configurations, in which case, the embodiment would be directed to address such a nerve condition. Furthermore, this montage can be supplemented with all or a portion of the autonomic configuration illustrated in FIG. 13 wherein the sympathetic nervous system can consequently be inhibited.

In one embodiment of the abovementioned system, the interface can be couple to, or continuous with, a second member through which a second, physical force is imparted to the target area Examples of such forces can include electrical current, thermal change, vibrotactile stimulus, light or other form of physical energy. In such an embodiment, the form of the interface would direct said physical energy directly to the target tissue or nerves. The interface may contain a form of circuit, conductive material or element specifically designed and intended to impart the desired physical energy In the case of light, for example, the conductive material may be a form of fiber optic fiber or light amplifying or dispersing material. In the case of thermal energy, the interface might contain a Peltier device or similar mechanism, which in some cases can actively changes temperature and secondarily imparts a temperature change to the target tissue. In another embodiment, the conductive material may be of a nature so as to retain heat or cold such that the interface can be prepared by heat or cold and subsequently retain this target temperature to deliver it to the intended target tissue over an intended amount of time.

The therapeutic interface can also include a combination of components intended to produce a combined benefit or series of combined benefits to improve the effectiveness of the overall therapeutic effect. In one instance, the interface can include a humectant or some other active or passive agent specifically intended to attract water to the skin surface, over the treatment area, which can serve one or more of the following three potential benefits, among others. Firstly, by attracting fluid to the treatment surface, there can be a sustained dilution of the therapeutic agent or agents being delivered to or through the skin surface to underlying tissue. Secondly, the presence of water can facilitate the transmission the therapeutic agents through the various epidermal and dermal layers. Thirdly, upon transmission through the aforementioned layers, the presence of water at the target tissue level can help to maintain an optimal dilution of the therapeutic agents, including maintaining an osmotic gradient to facilitate intracellular passage. The latter benefit is of particular benefit in the topical administration of therapeutic agents that are most therapeutic at low concentrations but should be presented on the skin surface in higher concentrations to account for attrition during transdermal or transcutaneous passage, particularly the topical compounds disclosed herein comprising varying amounts of dextrose, mannitol and/or bicarbonate. The use of one or more humectant ingredients reduced tackiness of sugar- or sugar alcohol-containing compound on the skin (e.g., making for a much more comfortable and appealing application) and also appeared to improved the efficacy of these compounds, which is likely attributable to the abovementioned factors and positive effects of increased water on the transcutaneous transmission of therapeutic agents.

Examples of humectants, which can serve in the abovementioned capacity in the therapeutic system, can include any one or any combination of Glycerin, Sodium PCA (sodium L-pyroglutamate), dimethicone, c30-45 alkyl methicone, c30-45 alkyl olefin, microcrystaline wax, polyethylene, silica dimethyl silylate. The humectant or humectant complex could also include silicone waxes, Butylene Glycol, Glycoproteins, collagen protein, Hexanediol CG, elastic protein, ethoxydiglycol, honey extract, jojoba, ceramide, sucrose cocoate, Glycerin (Glycerol), Sorbitol, PEG (polymerization of ethylene glycol), Propylene Glycol, Aloe Vera. Urea, Algae Extract, Sodium Hyaluronate (polysacharride), sucrose, glucose and other sugars and their derivatives, and Lanolin.

In further elaboration of potential components of the interfacing compound or combination of compounds intended to improve overall therapeutic benefit of the interface, a delivery system is another component intended to facilitate the delivery of the primary therapeutic agent or combination of agents, which may be a drug or combination of drugs. One example of a delivery system is an Organogelator, such as one or any combination of sterol, sorbitan monostearate, lecithin, and cholesteryl anthraquinone derivatives. Another component of the interfacing compound can include a penetration or permeation enhancer, which may include an amount of one or more of terpene, glycerine, propanediol, vegetable glycerine, hyaluronidase, polysaccharide, lipid complex or tetrasodium EDTA. An emollient can be included in the therapeutic system, which can include an amount of one or more of cold pressed plant oils (e.g. castor oil, squalane, jojoba oil, cocoa butter, shea butter, argan oil, octyl palmitate, sodium phytate, caprylic capric triglyceride, dimethicone, octyldodecanol, and tetrasodium EDTA).

In certain applications, it can be beneficial to present the abovementioned interface compound, in its various potential embodiments and iterations, as a topical compound, independent of a structured interface, rather, simply as a topical compound to be administered to the skin by hand or some other form of applicator. Example methods and systems of utilizing such compounds are described herein.

Therapeutic Device System and Method for Treating Nerves and Therapeutically Stimulating Central Nervous System Centers for Symptoms and Conditions

In certain conditions and patterns of symptoms topical therapeutic agents can be of benefit to reduce inflammation of superficial nerves and other tissue. It has also proven of benefit to use mechanical or physical methods to create movement of nerves or tissues surrounding nerves in order to liberate them from states of stasis or limited motion. Additionally, in certain conditions it can be of benefit to stimulate certain centers within the central nervous system to create a therapeutic response. Typically, therapeutic approaches and devices address the peripheral- or central nervous system, but this leads to incomplete relief in many cases or to using multiple approaches. Furthermore, in certain conditions, such as pain and headache, it can be of great clinical value to simultaneously address both peripheral and central components of these conditions. While a number of devices and therapeutic approaches to pain might utilize peripheral nerves to target the central nervous system and brain, some embodiments disclosed herein actually intentionally target peripheral nerves with the intention of specifically changing peripheral nerve (including superficial/cutaneous peripheral nerve) physiology (e.g., where creating a therapeutic change in the peripheral nerve itself is the therapy). This is particularly true in the field of conditions of the head and brain, such as headache, seizure, migraine, sinusitis, seasonal or environmental allergy symptoms, and sleep disorders. Some systems and methods disclosed herein can channel all three of these general therapeutic approaches in a single application.

The device system can include a container cavity, which contains a certain therapeutic agent. An example embodiment of such a topical therapeutic agent comprises an amount of sugar and/or sugar alcohol (e.g. dextrose and/or mannitol, respectively) or any combination of amounts of dextrose, mannitol, analgesic, anesthetic, humectant, permeation enhancer, terpene, emulsifier, and pH buffer (e.g., with an alkalizing effect). In some embodiments, each of the aforementioned component agents is selected for low comedogenic properties, optionally with a relatively score of around 2 or less out of 5 on a five point rating scale by an reputable and authoritative source. The therapeutic agent could also be any substance known to modulate nerve activity.

The container can hold a single chamber, holding a single therapeutic compound, or more than one chamber, e.g., holding one or more differing therapeutic compounds. A multi-chambered configuration can also intentionally house components of a certain therapeutic compound that are intentionally stored in separated form to maintain an optimal desired state that could be compromised by mixing the separated ingredients. Multiple chambers are intended to maintain separation of the contained therapeutic agents during storage There can be a number of benefits to maintaining such separation One such reason can include the ability to separate ingredients that offer therapeutic benefit in combination but are volatile and unstable when stored for prolonged periods of time (e.g, they would be expected to react in when combined in a single container). Another advantage is the ability to deliver higher quantities of therapeutic agents that are not easily or not possibly combined in a single therapeutic compound. This approach would negate the need to involve other formula stabilizing agents, which can potentially hold disadvantages, potentially including cost, undesirable side effects, unwanted effects on formula characteristics, or undesired bulking of total formula volume.

At the time of dispensing the therapeutic agents from their storage containers within the system body, the separated agents can be dispensed from the chamber in sequence or in combination, potentially including in a sequenced application (e.g., one or more components delivered one-at-a-time), in a staged application (e.g., one or more at a time, separated by a period of time, which might be for example, morning and night), in a combined application (e.g., wherein one or more components separated by storage are combined at the time of dispensing to the user). One of the benefits of this configuration is that the therapeutic compound can use fewer components that would otherwise be used for a topical cream or gel, as in some cases the compound does not need to hold together like a cream. This device allows for the compound to be more fluid. This allows for a higher concentration of therapeutic agents as well as a less costly composition. This can result in a less harmful composition of certain topical therapeutics owing to the reduced need for ingredients with the purpose of maintaining texture of the topical therapeutic.

The mechanism that dispenses the contents of the storage compartments, can be actuated by a number of potential mechanisms. For example, this can include a simple increase in intra-compartment pressure by deformation (including by way of the hand) of readily deformable container walls. In such a mechanism the user would simple squeeze the container to dispense contents. Another potential dispensing mechanism might be a pumping mechanism, such as having an advancing member within the chamber that acts to reduce the intra-compartment volume and increase pressure in a manner resembling a plunger, wherein one or more walls of the compartment advance so as to reduce compartment volume in an advancing manner so as to increase pressure to drive the dispensing of contents. This plunging mechanism can be actuated or facilitated by a secondary component such as a spring, a magnet, or mechanical mechanism, which assists with the advancement of the chamber-volume-reducing mechanism. Such a mechanism can be used to increase the velocity of the fluid being dispensed so as to improve the delivery of the fluid contents through the outer layers of the skin. The velocity achieved by the mechanism can be a high velocity so as to facilitate delivery to deeper tissues, which might not have been accessible by way of a conventional topical application method (e.g. rubbing in cream). Yet another mechanism can be through the use of automation that utilizes a form of automated mechanism to increase pressure within the container space (e.g an electrical or electromagnetic linear actuator). The user might actuate this automated mechanism (e.g., electronically via a button or other user input element) at the time that dispensing of therapeutic contents is desired.

In a certain embodiments the device system could utilize a shared- or secondary-pneumatic generator that aids in transdermal delivery of therapeutic agents by generating pneumatic pressure pulses that serve to push a therapeutic topical through the skin surface, in some cases whilst simultaneously imparting a soothing and stimulatory sensation to the skin and underlying tissues. By “shared”, the intention is that the pneumatic dispensing mechanism can also serve as the therapeutic pneumatic pulsing mechanism at the time of application. “Secondary” is intended to mean that the dispending pneumatic component is separate from the therapeutic pulsing mechanism. The therapeutic pulsing mechanism may take the general form of an electromagnetically driven piston, electromagnetically controlled linear actuator or similar mechanism.

The housing of the device can also hold a power source to power this mechanism. These mechanisms might be actuated by the user in the form of one or more buttons, switches, dials, or other user input elements, situated on the container surface, which may be color-, texture-, or visually coded (including for example by some form of digital, LED or electronic screen, or touchscreen), which indicates to the user which substance is being delivered by activation of a particular button or may even impart some form of message or instruction to the user. In some embodiments, separate dispensing mechanisms (e.g., pumps) can be used for the different compartments, and can be used to dispense the contents of the compartments separately or together, such as based on user input or the desired application.

Now to further describing the therapeutic device system. The portion of the device contacting the user's body can include a variety of potential forms and mechanisms intended to dispense a single or multiple therapeutic compounds onto the skin There can be some form of single or multiple channels linking the interface component with the internal storage compartment. One potential interface or contact surface form is a flat shape wherein a flat surface is penetrated by one or more channels through which a fluid medium may pass. The contacting surface can be textured or comprise some form of irregular surface so as to increase friction with the skin so as to improve grip in order to allow the user to better manipulate the skin and underlying tissues, especially superficial nerves. The utility of this mechanism is that by contacting superficial tissues and allowing the user to move (e.g., cyclically maneuver) the contact surface whilst in contact with the skin, the therapeutic effect is the liberation and mobilization of body tissues in the region, especially the superficial nerves, which can reduce mechanical irritation of nerves by liberating them from surrounding tissues. Another benefit is that with this mechanical friction, the protective, tight junctions of the outer skin layers can be mechanically disrupted, markedly improving delivery of the therapeutic agent to the deeper layers of tissue. This effect is markedly enhanced efficiency, clinical effectiveness, and user comfort by the simultaneous use of therapeutic topical compounds that target nerves and nerve symptoms and penetration-enhancing agents. This can be especially true of compounds containing any combination of an amount of: dextrose (or other sugars), mannitol (or other sugar alcohols), pH buffer (e.g., alkalizing agent), polyphenol, cannabinoid (e.g. Cannabitriol (CBT), Cannabinol (CBN), Cannabigerol (CBG)), anesthetic, analgesic, anti-inflammatory, permeation enhancer, humectant, and emollient.

While the abovementioned embodiment contains a static surface, the intended therapeutic effect can be enhanced in certain instances by the use of a dynamic interface surface. Toward this end, one embodiment of the interface component can contain one or more roller balls, which dispense container contents as they roll across the skin surface. Yet another embodiment can include a mechanism that moves or deforms in order to arrest an amount of superficial tissue, for example a deformable suction cup, constricting-, skin-gathering- or pincer-mechanism.

In certain embodiments the interface- or skin-surface-contacting component may house a mechanism to impart a secondary physical energy (e.g., energy that is not directly imparted by the user's hand), which can include vibration, oscillation, percussion, electrical current, thermal change (e.g., heating or cooling), light, sound, ultrasound or magnetic energy. This mechanism can offer a number of important benefits and serve multiple purposes to contribute to the effectiveness of the device system. Firstly, it can stimulate receptors and peripheral nerves so as to create afferent signals to stimulate the central nervous system. Secondly, this energy can create a form of response in the target tissue and skin to facilitate transmission of therapeutic compound to target structures (e.g. increasing the permeability of the skin). Thirdly, with the application of various, simultaneous forms of energy an analgesic effect is produced, which allows for the application and delivery of a topical therapeutic agent, which might have otherwise been too painful or uncomfortable to deliver.

An embodiment of this mechanism is a contact surface platform that contains an oscillating- or vibrating component, which can deliver a single rate oscillation frequency, variable frequencies or selectable frequencies, which might be selected by the user or by electronic device processor.

In another embodiment of this mechanism the contact surface platform that houses an oscillating-, electrical- or vibrating component, is actuated by a mechanism wherein pressure with the target surface causes depression of the contact surface housing toward the body of the device, where such action activates the dispensing of any of the aforementioned physical energies such that while the depressed station is maintained, a pre-determined energy pattern is delivered via the contact surface. When the activating pressure is released, the dispensing of energy is ceased and the device returns to inactive state.

Another embodiment, consistent with the above-described characteristic of delivering electrical energy, can include a contact surface (interface) platform that acts as an electrode against the skin wherein it becomes a conduit. In one embodiment, this electric energy will be in the form of a direct current (like a direct monophasic current delivered by galvanic) in the range of about 0.3 microamperes to about 5 milliamperes, however, the electrical energy transferred can be of any form deemed therapeutic. The contact surface can be electrically conductive, and can be coupled to a power source so that the contact surface can deliver the electrical current A surprising finding produced by the introduction of this form of current was that users were able to tolerate a greater degree of pressure as well as vibration on symptomatic superficial nerves. Thus, mechanically liberating nerves required less time and could be achieved more thoroughly by the use of more pressure. Furthermore, with the use of an aforementioned topical compound, containing dextrose or mannitol for example, there was a unique and highly favorable relief experience on the part of the user. Onset of relief was faster than with topical (and oscillating mechanism) alone and typically much longer in duration than with electrical current alone. This effect was observed in multiple body parts including, head (in multiple areas and across multiple symptoms), lower back, knee, thigh (posterior), shoulder and foot.

In areas such as torso, lower back, hips and limbs, it may be beneficial to utilize a larger interface component (e.g., forming a larger electrode surface), while in areas such as the neck, head and face, a smaller interface component can be indicated. As an example embodiment, one larger interface component, formed like a “rolling pin” (cylinder) might be about 3 inches long and about 1 inch in diameter. In another example embodiment, intended to appeal to smaller body surfaces, this cylinder can be approximately 1 centimeter in length with a diameter of approximately 0.75 centimeters. As previously mentioned, this interface component may take any form including, sphere, multiple spheres, flat surface, textured surface, hoop or loop (wherein the center is hollow and the circumference is a solid component). A rolling pin interface can have a length of about 0.5 cm, about 1 cm, about 1.5 cm, about 2 cm, about 3 cm, about 4 cm, about 5 cm, about 6 cm, about 7 cm, about 8 cm, about 9 cm, about 10 cm, about 12 cm, about 15 cm, or more, or any values or ranges therebetween. The rolling pin interface or a roller ball interface can have a diameter of about 0.1 cm, about 0.25 cm, about 0.5 cm, about 0.75 cm, about 1 cm, about 1.5 cm, about 2 cm, about 2.5 cm, about 3 cm, about 4 cm, about 5 cm, or more, or any values or ranges therebetween. Other sizes could also be used. The flat interface can have a surface area of about 0.25 cm², about 0.5 cm², about 1 cm², about 1.5 cm², about 2 cm², about 2.5 cm², about 3 cm², about 3.5 cm², about 4 cm², about 5 cm², about 7 cm², about 10 cm², about 15 cm², about 20 cm², about 25 cm², about 30 cm², about 40 cm², about 50 cm², about 60 cm², about 70 cm², about 80 cm², about 90 cm², about 100 cm², or more, or any values or ranges therebetween, although other sizes could also be used.

In a certain embodiment, the interface component may house a mechanical component capable of imparting a tactile oscillating or vibrational energy. This energy may be at a fixed rate or variable rate, fixed amplitude or variable amplitude. The rate of vibration may vary from around about 0.5 Hz-about 200 Hz, such as at about 1 Hz, about 5 Hz, about 10 Hz, about 20 Hz, about 30 Hz, about 50 Hz, about 75 Hz, about 100 Hz, about 125 Hz, about 150 Hz, about 175 Hz, about 200 Hz, or more, or any values or ranges therebetween, although other values could be used. In one embodiment, the oscillation rate may be adjustable through a component (e.g., a user input element) in the controller portion of the device. In a certain embodiment the interface component may also house a thermal energy generator (e.g., for heating and/or cooling) such that the interface is capable of delivering thermal and kinetic energy simultaneously in variable configurations In an example embodiment, the interface component contains a channel or similar mechanism whereby a therapeutic compound can be delivered to the body surface and whereby the therapeutic energy delivered by the interface component assists in the delivery (e.g., transcutaneous delivery) of the therapeutic compound.

At times, it may be of benefit to deliver a positive pressure to target body tissue and nerve tissue so as to apply therapeutic friction, which may be imparted in association with or without therapeutically synergistic forces. Toward this therapeutic end, a general device and method concept embodiment is hereby offered, which for purposes of explanation herein will sometimes be referred to a “positive pressure friction system”.

Whilst the above concept is termed for purposes of explanation “positive pressure friction system”, primarily due to the fact that the term is offered as a relative term to other listed embodiments and the initial contact with the body surface might be characterized as positive in nature, it should be noted that the actual physical forces applied to the target tissue may be positive (e.g., pressing on the tissue), negative (e.g., pulling on the tissue), alternating (biphasic including positive and negative), cyclical, sustained, repetitive, variable, high-rate, low-rate or any other pattern of intensity, frequency, amplitude or duration deemed appropriate for the therapeutic objective. In an example embodiment, whilst in a particular therapeutic session a positive pressure is maintained with the target tissue, the interface component imparts a second energy type or a secondary kinetic energy In the case of kinetic energy, this may be directed in a plane or direction that is substantially parallel with the positive pressure vector, substantially perpendicular to the positive pressure vector, or in any other vector or combination of vectors, or other motion patterns such as circular motion. This secondary motion (e.g., vector) can mechanically liberate target, superficial nerves, and in some cases can also impart a stimulating stimulus to target nerves and receptors.

At times, there may be benefit derived by the application of a persistent negative pressure to the target tissue, which may be imparted with or without synergistic therapeutic forces. Toward this purpose, another embodiment device and method, is described, which for purposes of description hereafter will be termed “vacuum dispenser concept”. This approach can be designed so as to impart a traction effect on the skin, underlying nerve tissue and target tissue. This is achieved by effecting a negative pressure state in the environment just superficial to the skin so that the skin and underlying tissues, especially superficial nerves, passively move into or towards the negative pressure space, generally describable as a vacuum effect. This allows a firm contact with the skin and tissues such that the user can apply a form of mechanical energy or manual manipulation to the target tissue via the device, which can impart a therapeutic benefit, including but not limited to mechanical liberation of symptomatic and/or painful superficial nerves. This configuration generally includes a formed head, optionally resembling an inverted cup and tubular form. In order to describe this component, the example of an inverted cup will be used. On one end, the body-contacting surface (or rim of the cup) is open to the environment, in some cases with a reinforced rim (e.g., primarily to better impart a comfortable seal on the skin but also to serve as a platform for other components that can serve to impart other forms of energy (see below).) The other end (termed “device end” from here), is essentially sealed but for a channel wherein it is penetrated. This channel serves as a conduit for the fluid (optionally air but also potentially air and fluid combination) that changes the pressure state within the cup, once it is sealed on its open end by contact with the body.

The vacuum dispenser concept contact platform (or cup to continue with the description above) can house a valve on the device-end such that with actuation of a dispensing action on the part of the user or device, the valve could allow a certain amount of fluid to pass through the valve into the cup and skin surface. The fluid dispensed can include therapeutic agent or simply gas, air, or fluid that is intended to change the pneumatic state of the cup when in contact with the skin. In a certain embodiment this valve can be oriented or configured so as to lessen the amount of negative pressure, such as by permitting an amount of fluid (e.g., air) to enter the device (e.g., the cup). In some embodiments, the valve can be oriented or configured so as to increase the amount of negative pressure, such as by drawing an amount of fluid (e.g., air) out of the device (e.g, the cup), which can create and/or maintain and/or increase a vacuum. The valve can allow for increasing the negative pressure (e.g, to produce a stronger vacuum) in the cup environment with sequential cycles of a certain mechanism (e.g., a button, knob, or other user input element) so as to increase the tension on the skin surface. In some embodiments, the device can increase the negative pressure in response to a first user input (e.g., via a first valve drawing an amount of fluid out of the device), and the device can decrease the negative pressure in response to a second user input (e.g., via a second valve permitting an amount of fluid into the device). By providing inputs, the user can increase and/or decrease the negative pressure to a desired level. In another embodiment, the device-end of the cup can contain two channels or two valved conduits wherein one is dedicated to the transmission of topical agent, whereas the other is dedicated to altering the state of pressure (e.g., increasing and/or decreasing) within the cup. In some cases, multiple valves can be used for altering the state of pressure, and yet another valve can be used for administration of a topical agent or composition. These configurations can be designed to support the therapeutic method described below such that it allows for dispensing of a topical therapeutic agent, which can also serve to enhance the seal of the cup against the skin, and also allows for changing pressure of the internal environment of the cup, for example so as to impart incremental traction to the tissue sealing the open end of the cup. This mechanism could be effected through the previously mentioned, multi-chambered configuration of the device body wherein one cavity would be empty (e.g., not containing a topical composition, but potentially containing a fluid medium, gas, or environment air) configured to create a movement of air through the aforementioned valve as this cavity is diminished and expanded in volume. One embodiment of this concept can be actuated simply by a firm but deformable cavity wall that can be depressed to expel air (just prior to application with the skin) then allowed to reform when the cup is sealed by the skin, thereby imparting a vacuum effect within the cup as the cavity wall reforms. This effect can be repeated through several cycles as fluid or air is allowed to quickly escape (with depression of the cavity wall) through the sides of the contact surface of the cup, then as the cavity reforms again, more traction is imparted to the skin. This effect can be facilitated by a valve (e.g., in the cavity wall) that allows for expulsion of fluid or air A check valve or one-way valve can be used in another embodiment, a vacuum can be imparted to the cup via a bulb, or a manual- or automated pump attachment, specifically configured to alter the internal pressurized state of the cup as it is in contact with the skin. This pressurizing mechanism can in some embodiments be actuated through cyclical phases of positive and negative pressure so as to impart alternating phases of pressure and vacuum to the body tissue in a vibrating or oscillating fashion, thereby imparting a primary- or synergistic therapeutic effect to target tissue.

In a certain embodiment of the vacuum dispenser concept, traction or depressurizing contact platform, a certain portion of the contact surface, which may be an electrified out perimeter, can conduct an electrical current configured to impart a certain additional or synergistic energy. An example embodiment of this component can include an electrical current generator, which imparts a certain electrical waveform, which in a certain example, includes a mild, negative monophasic galvanic current. Furthermore, some embodiments, can include, as part of a topical therapeutic fluid, gel, paste, cream, hydrogel or medium of particular viscosity, a component configured to facilitate transfer of electrical current, for example silver or silver chloride. The presence of this gel or other medium can have a particular synergistic effect that lowers the required intensity of electrical energy transfer, making the stimulating electrical current more comfortable and effective to administer.

In consideration of the therapeutic application of the abovementioned vacuum dispenser or pressure adjustment concept, a therapeutic method is described as follows: 1) a certain target tissue is identified; 2) the device-skin contact surface (e.g. cup) is contacted around or on the target body tissue, 3) an amount of topical therapeutic is (optionally dispensed), 4) the device is actuated so as to impart a vacuum force on the target tissue; 5) (optionally) an amount of additional energy (e.g. electrical current, vibrational energy or therapeutic light) is dispensed to the target tissue; 6) whilst in contact with the skin and target tissue (via altered pressure within the device-skin contact mechanism) the device is maneuvered so as to manipulate the target tissues; 7) at conclusion of prescribed time period the application may be ceased or repeated as dictated by the particular case.

In a particular embodiment of this method, the target tissue is symptom-generating or painful superficial nerves and the intention for the use of the device is to therapeutically appeal to these nerves in one or more of three ways: firstly, with the (optional) use of electrical current, a modulatory effect is provided that decreases adverse symptomatic traffic through the peripheral nerve with a consequent positive effect on central (spinal cord, brainstem or brain) centers. Secondly, the mechanical forces delivered serve to liberate restricted or entrapped nerves as well as create movement in otherwise restricted surrounding tissue. Thirdly, with the administration of a topical therapeutic (optionally but not limited to a medium containing any combination of mannitol, dextrose, alkalizing agent, TOR- or m-TOR modulator, polyphenol, fruit- or plant extract (or combinations thereof), cannabinoid, humectant, permeation enhancer, analgesic agent, anti-inflammatory, tissue regenerative agent, electric current facilitator) serves to facilitate cup seal; provide enhanced healing and symptomatic reversal of target tissue, especially nerves; impart a synergistic therapeutic benefit to targeted nerves and surrounding tissues.

The abovementioned system and general treatment method can be utilized on the face and head to treat or affect conditions or states of the head but might also be used at any other location on the body where a combined peripheral- and central-therapeutic effect may be desired. An example can include post-surgical rehabilitation of a joint (e.g. hip, knee, shoulder, spine or wrist) where it is desired to alleviate peripheral nerve symptoms (sometimes caused by surgery yet undertreated) whilst activated endogenous analgesic systems of the spinal cord (e.g. pain gating), brainstem (e.g. conditioned pain modulation), or brain (e.g. basal ganglia or thalamic mechanisms.) Yet another example can include the treatment of bodily pain from tendon-, muscle- or joint conditions where guarding or limited motion due to pain or injury can lead to undesirable under-stimulated states of the central nervous system, which would augment pain and disability. In such cases, the delivery of simultaneous peripheral- and central-nervous system stimuli in concert with topical administration of topical therapeutic agents could markedly accelerate recovery of function in a variety of physiologic domains.

A Device System for Imparting Mechanical and Electrical Energy to Target Superficial Nerves

In a certain embodiment multiple nerve points can be addressed in an efficient manner wherein a particular device is utilized which is intentionally designed to deliver 4 components of the treatment method:

1) an interfacing surface intended to make contact with the overlaying skin, which can take the form of a flat interfacing surface but may also be of any other form intended to contact or arrest the tissue. These potential forms can include a “rolling pin” (cylindrical roller), roller-ball, a multi-roller-ball (more than one interfacing mobile spheres), a suction surface (intended to exert a negative pressure on the skin in order to traction the superficial tissues overlaying the target nerve), an active pincer- or similar gripping mechanism, or a textured surface (suited to increase friction and grip with the skin).

2) a channel connecting one or more therapeutic-agent-storage-compartments with the body-interfacing surface of the device, delivering a therapeutic, compound on the interfacing surface and/or skin overlaying the target nerve.

3) use of a topical compound intentionally targeting symptomatic nerves or tissues (which may include those with an anti-inflammatory, analgesic, anesthetic, metabolic, or other therapeutic properties) and/or compound described herein compromising an amount of sugar or sugar alcohol (e.g., one or both of dextrose or mannitol with total volume of around 3-50%) and a vehicle compound of some form, including a vehicle, excipient and/or humectant component. This compound can comprise a relatively alkaline medium and an amount of one or more anesthetic or analgesic agents. In a certain example embodiment, the therapeutic compound may also contain an amount of analgesic, anti-inflammatory, TOR or m-TOR modulator, polyphenol, combination of one or more plant or fruit extracts, peptide- or peptide fragment, cannabinoid, regenerative therapeutic agent, or any topical compound known to impart a therapeutic response in target tissue. The topical compound may include anti-inflammatory, analgesic, anesthetic, metabolic, or other therapeutic effect. In one embodiment, the therapeutic compound may be delivered in a relatively alkaline medium, including a vehicle, excipient and or humectant or in another embodiment, an amount of sugar or sugar alcohol, an amount of alkalizing agent, an amount of topical vehicle, including excipient, vehicle and/or humectant, and an amount of one or more anesthetic or analgesic agent.

4) a form of physical energy delivered to the target nerve through or in combination with the therapeutic compound described in #3. This physical energy can include one or more of vibration, oscillation, friction, electrical current, sound, ultrasound, thermal energy (heat or cold), light, laser light or magnetic energy. The energy form can be electrical (direct, continuous, negative, monophasic) or laser light but can take any form that is synergistic to the therapeutic agents and overall therapeutic approach. This therapeutic energy can be imparted by a trigger mechanism in the interface component of the device system. One example of this trigger mechanism can involve a spring-actuated reloading mechanism wherein the default position of the interface component is in the “off” position (therapeutic energy is not being dispensed via the interface component), however, when the interface component is depressed by the skin contact surface, the therapeutic energy component of the system is activated.

Consistent with the therapy method described above for the treatment of peripheral nerve symptoms and conditions, the device system can be directed at particular montages or symptomatic groups of one or more superficial nerves of the body.

A Device System for Imparting Chemical. Mechanical and Electrical Energy to Target Superficial Nerves of the Nasal Structures and Brain

In certain conditions, especially in pain, behavioral disorders and trauma (e.g. post-traumatic syndrome, depression, anxiety, seizure, traumatic brain injury, autonomic disorders, insomnia, multiple chemical sensitivities, headache disorders, alzheimer's disease), the brain's Limbic System is involved. The olfactory bulb is a direct extension of the telencephalon (e.g., including the temporal lobe and/or amygdala). Due to this relationship, stimulation of the olfactory bulb via certain stimuli, e.g., smell, can have a therapeutic effect on the brain. It has also been shown that nasal (not oral) inspiration (more so than expiration) has a particular therapeutic signature in the brain.

The superficial nerve projections of the external and internal nasal structures serve both somatic and autonomic systems of the brain. These can become locally irritated (e.g., inflammed or sensitized) as well as adversely or positively affect their central targets in the brain.

In certain embodiments, the abovementioned areas of brain and multiple superficial nerve points within the nasal structures can be therapeutically addressed via a single device platform. The device platform can deliver 4 components of the treatment method.

1) An interfacing surface intended to make contact with the superficial tissues of the nasal passages. An embodiment of this component is in the approximate form of an umbrella-electrode configuration wherein this platform applies an outwardly directed force against the walls of the nasal passages to maintain contact against a section of the nasal passages, intended to deliver electrical energy. This structure houses one, two or more electrodes, which are intended to deliver electrical energy to the walls of the nasal passages and underlying tissues, especially the superficial nerves. While this component is intended to deliver electrical energy, the system might also be modified to deliver vibration, oscillation, sound, thermal (e.g., warmth or cold), light (e.g. laser, infra-red, or visible light (e.g., including red and/or green)).

2) A channel connecting one or more therapeutic-agent-storage-compartments with an dispensing aperture or opening, which is intended to deliver an amount of a therapeutic compound, which may be gel, liquid, gas, powder, in the form of a spray upwardly and outwardly into the nasal passages. This channel may or may not involve one or more valves. Therapeutic agent is expelled through this channel when pressure builds in the container component of the device system, thereby driving opening of the valve and release of the therapeutic agent.

3) Use of a therapeutic compound, which may be of a nature described in this document, or of entirely different composition, intended for any therapeutic purpose. This therapeutic compound, may intentionally target symptomatic nerves or tissues (which may include those with an anti-inflammatory, analgesic, anesthetic, metabolic, or other therapeutic properties) and/or compound described herein compromising an amount of sugar or sugar alcohol (e.g. one or both of dextrose or mannitol with total volume of around 3-50%) and a vehicle compound of some form. This compound can comprise a relatively alkaline medium. It may contain an amount of one or more anesthetic or analgesic agents. In a certain example embodiment, the therapeutic compound may also contain an amount of analgesic, anti-inflammatory, TOR or m-TOR modulator, polyphenol, combination of one or more plant or fruit extracts, peptide- or peptide fragment, cannabinoid, regenerative therapeutic agent, or any topical compound known to impart a therapeutic response in target tissue.

The therapeutic compound can include an amount of an agent intended to enhance the transmission of electrical current to the target tissues, such as zinc or zinc oxide. An advantage of this therapeutic device system is that the release of the therapeutic medium with conductive elements affords the transmission of electrical current to regions of the nasal passage, which would otherwise be unreachable by direct contact. This also includes the olfactory bulb of the brain itself.

The therapeutic compound may also involve a psychotropic agent wherein the agent and the overall function of the device system would work synergistically to bring about desired changes in the networks in the brain associated with emotion, cognition, behavior and sensory (including pain) modification. This particular embodiment of the therapeutic method is expected to have particular utility in the field of addiction- and post-traumatic treatment.

4) In a particular embodiment, intentionally developed to stimulate the olfactory bulb and its associated brain structures, there may be an amount of an odorant, which may be a naturally-occurring substance (e.g. an amount of edible foodstuff, fruit, flower, essence) or a processed substance (e.g. terpene-derivative such a d-limonene, a-pinene, limonene, myrcene).

5) A form of physical energy delivered to the target nerve through or in combination with the therapeutic compound described in #3. This physical energy can include one or more of vibration, oscillation, friction, electrical current, sound, ultrasound, thermal energy (e.g., heat or cold), light, laser light or magnetic energy. The energy form can be electrical (e.g., direct, continuous, negative, monophasic) or laser light but can take any form that is synergistic to the therapeutic agents and overall therapeutic approach. This therapeutic energy can be imparted by a trigger mechanism in the interface component of the device system. One example of this trigger mechanism can involve a spring-actuated reloading mechanism wherein the default position of the interface component is in the “off” position (e.g., therapeutic energy is not being dispensed via the interface component), however, when the interface component is depressed by the skin contact surface, the therapeutic energy component of the system is activated.

The therapeutic effect of an application using this device embodiment might be enhanced by instructions to apply stimulus over prolonged inhalation through the nose wherein the user will be exposed to simultaneous, synergistic effects of odorant, electrical, therapeutic compound exposure, which is expected to positive affect peripheral nerves, and central nervous system. While the preferred method is synchronization with inhalation through the nose, the method might be applied in synchronization with any phase of respiration through mouth or nose.

As described herein, this therapeutic method is applicable to a range of conditions but has particular application as a method of graded exposure for individuals with sensitivity to odors or chemicals, wherein graded amounts of odorant might be delivered in conjunction with stimuli intended to syngerstically create plastic changes in regions of the brain typically provoked by these stimuli in these type of disorders That is, this method is expected to reduce chemical and odorant sensitivities.

In one embodiment of the system, the device platform can include an oral attachment to deliver the above synergistic combination of forces to the structures of the mouth.

In another embodiment, a component that fits in the mouth can be used to allow the user to swallow or inhale against some form of resistance in order to stimulate the autonomic nervous system to enhance the therapy. In one example of such an application, the user would inhale through the nose (e.g., and receive any combination of the abovementioned stimuli), followed by inhalation or swallowing against some form of resistance offered by the oral component of the system. This application might be repeated in multiple cycles.

Method for Treatment of Body Symptoms and Pain by Energetic Topical Compound Delivery System

The following details a treatment approach for pain and body conditions, for example involving the abovementioned device system.

Step 1. One or more therapeutic compounds, intended for topical delivery, can be prepared and stored in the device system in one or more storage containers of the device. These agents can be intended for application in a sequence of applications or in one more or applications wherein one or more of the stored ingredients are administered in a combination on the skin surface.

Step 2: One or more symptom-generating nerves or tissues is identified (localization of nerves or tissue can be achieved by one or more of palpation; negative or positive pressure on overlying tissue; visual scanning for diagnostic indicators; guidance by user history; movement of limbs so as to impart compression or traction; anatomical understanding on the part of the trained clinician who might recognize the symptomatic area as being associated with a particular nerve or combination of nerves). Isolation of these target tissues can occur before use in a prescriptive fashion and might not be a component of every application session. In a certain embodiment, the device may contain a sensor for sensing electrical impedance on the skin surface, allowing for detection of areas of high vs. low tissue impedance in order to more accurate isolate superficial nerves (as areas with nerves close to the surface can exhibit a different impedance to other areas of tissue).

Step 3: The device-body-interfacing component (typically the tip) is applied to the skin overlying the target nerve or tissue (the skin may be the target tissue). Having made contact with the skin, the topical therapeutic compound is dispensed from the container to the skin. In some instances the mechanical force and/or accompanying energy may precede or follow the administration of the topical therapeutic material from the container. In some instances, some topical agent may be dispensed to the device interface prior to contact.

Step 4: Therapeutic force or energy is imparted to the skin and body surface by the user or by a mechanism intrinsic to the device system. In an example embodiment of this method, the force imparted by the system is vibration or cyclical friction, as the user maintains a gentle superficial-to-deep vector of pressure on the superficial tissue surface. In another, different embodiment, the force imparted is pulsed pneumatic pressure, which may be modified environmental air, stored gas or any other form of fluid flow, as discussed herein.

One benefit of these particular embodiments is that they each provide a gentle, stimulatory effect to the target tissues, whilst driving therapeutic compounds into deeper tissues in less time compared to manual applications. Furthermore, the applied energy can be synergistic in terms of stimulating target structures of the central nervous system (CNS). Other forms of cyclical energy pulses can be administered to provide waves of “pushing” energy to drive agents from the skin surface to target tissues. These pulses of energy can also serve to activate some relatively inactive state of the target tissue or therapeutic compound being delivered by the device system. Other method embodiments can include for example, the administration of light (e.g. “cold laser”), percussion, thermal energy, ultrasound, magnetic pulses, radiofrequency, wave pulses (e.g. shock wave), electrical energy and/or pneumatic-, gas- or air pulses. This therapeutic method can be intentionally modified to suit particular regional or condition-based characteristics of the target body tissue or symptom condition being addressed by the therapeutic method. Specific examples are listed herein.

Therapeutic Topical Compound and Method for Treating Pain and Body Symptoms

The following described device system can deliver a particular therapeutic topical compound. What follows is a description of such an example compound, which can be delivered by the aforementioned device system or by any other method or device that can deliver a therapeutic compound to the human body, including by hand or interface with the skin. A need exists for a topical compound, aimed at mitigating neurogenic inflammation that is appropriately developed for application to the face In this regard, it is important that the therapeutic compound avoid or reduce comedogenic agents (pore clogging). Furthermore, there is a growing awareness among consumers as to toxicity of conventionally used ingredients in topical therapeutic compounds. In response, a compound is described, which offers a balance of therapeutic efficacy (especially in the treatment of pain of neurogenic inflammation) and limitation of adverse side effects, such as pore clogging.

One embodiment of the therapeutic compound would be comprised of a sugar or sugar alcohol (e.g., in a range of about 1-40% by weight) (for example, dextrose or mannitol); an alkalizing agent that is configured raise the pH of the compound to between pH 7.5-10 (potentially including one or more in any combination of L-Arginine, Sodium Bicarbonate, and sodium citrate), an emollient, which includes a humectant, including any combination of glycerine, caprylic triglyceride, c30-40 alkyl methicone, c30-45 alkyl olefin, microcrystalline wax, polyethylene (synthetic wax), silica dimethyl silylate, xanthan gum, sodium hyaluronate, ceresin wax; a penetration enhancer, which can include any combination of vegetable oil (from Argan oil, Jojoba oil, and shea butter), terpene (such a d-Limonene), aloe, Arnica, and tetrasodium EDTA, and a natural preservative or antimicrobial, including any combination of naticide, radish root, phenoserve and Optiphen. The compound can also include (in any combination) an amount of polyphenol (e.g. Pterostilbene), combination of one or more plant or fruit extracts, a cannabinoid (e.g. Cannabitriol (CBT), Cannabinol (CBN), Cannabigerol (CBG)), and Vitamin D.

The abovementioned therapeutic compound can also be presented with alternative penetration enhancers that meet the specified goal of avoidance of synthetic and harmful ingredients, whilst maintaining the intended purpose. Such ingredients can include any combination of Lecithin, Terpenes or Terpenoids; Menthol (e.g., 0.25%-20/6 by weight); Camphor (e.g., 0.25%-20% by weight); Limonene (incl d-limonene) (e.g., 0.25%-20% by weight), Myrcene, Pinene (a-pinene, B-Pinene), p-Cymene, Carvone, Borneol (e.g, 0.25%-20% c by weight), Labrasol, and Transcutol (including derivatives from Cannabis plant; a-humulene, Linalool. Myrcene, Terpinolene, a-Bisabolol, B-Caryophyllene, Nerolidol, B-Caryophylle), Eugenol, dimethyl sulphoxide, acetone, propylene glycol and tetradihydrofuryl alcohol.

In some cases, a cream can contain mannitol as the primary active ingredient, for example wherein the ingredients include 30% mannitol, propylene glycol, purified water, isopropyl palmitate, caprylic/capric triglyceride, ceteareth 20, cetearyl alcohol, glyceryl stearate, PEG-100 stearate, dimethicone, octyldodecanol, lecithin, ethylhexylglycerin, and phenoxyethanol. While this formula can be used for topical application in some applications, it was found to be problematic in two primary ways, especially when applied to the skin of the face Firstly, it contained comedogenic (blackhead, pimple and pore-clogging) properties, and secondly, it contained contentious ingredients not suitable for marketing this formula as universally safe. The comedogenic properties of this formula render it essentially unusable on the face for any practical amount of time (e.g., users will have to face the likelihood of undesirable cosmetic consequences with use, especially repeated use). Specifically, it appears that at least isopropyl palmitate and ceteareth 20 are likely the primary comedogenic culprits. For this reason, these ingredients were experimentally substituted in various iterations with vegetable oils (including, Shea butter, Argan oil, Aloe vera and/or Jojoba oil), in place of isopropyl palmitate, with positive benefits and Xanthan gum, in place of ceteareth 20, with positive effects. Furthermore, a number of ingredients were found to be undesirable (and consequently unmarketable to a health-conscious population) due to a growing body of concerns within the field regarding their toxic and auto-immunity provoking properties, namely PEG-100 stearate and phenoxyethanol. Phenoxyethanol was experimentally replaced, in various iterations with naticide, radish root and/or pterostilbene, each of which appeared to support normal function of the topical compound whilst offering a less contentious and more appealing ingredient for health-conscious consumers. PEG-100 stearate was eliminated from iterations of experimental formulae with no adverse consequence on functionality and characteristics.

Thus, in consideration of the abovementioned experimentation with a topical, intentionally developed or configured to treat pain and inflammation, especially pain and inflammation caused by neurogenic inflammation, with a low-risk profile for adverse side effects and low-risk for comedogenic provocation, a compound and embodiment is hereby described containing about 5-40% Mannitol (or about 5-40% Dextrose), in an excipient, vehicle or base containing an (optional) amount of analgesic or anti-inflammatory (including but not limited to menthol, camphor, cannabidiol (CBD) or other canabinoid, wintergreen, polyphenol, or salicylate), an amount of water (e.g, purified water), an amount of propylene glycol, an amount of relatively low-comedogenic potential oil (e.g., comprising one or more of aloe vera, shea butter, argan oil or jojoba oil), an amount of caprylic/capric triglyceride, an amount of xanthan gum, an amount of texture enhancer (e.g. cetearyl alcohol), an amount of glyceryl stearate, an amount of emollient (e.g., dimethicone), an (optional) amount of octyldodecanol, an amount of lecithin, an amount of ethylhexylglycerine, an amount of anti-microbial or preservative (incl. one or more of naticide, radish root, pterostilbene, polyphenol). With respect to anti-microbial agent, Pterostilbene (a polyphenol) was found to be particularly favorable and synergistic as a component of the therapeutic compound, which supported therapeutic action as well as anti-microbial properties to counteract microbial proliferation on a glucose substrate. Furthermore, it was found that using a fruit- or plant-extract was a stable and cost-effective manner to deliver TOR-modulators and polyphenols. The following are noted as particularly effective for this application: Rhodiola Rosea Extract (e.g., CAS No: 97404-52-9), Kakadu Plum Extract (e.g., CAS No: 56-81-5, 7732-18-5, 1176234-54-0, 122-99-6), Acat Fruit Extract (e.g., CAS No: 879496-95-4), Salvia miltiorrhiza Bunge (danshen) (e.g., CAS No: 90106-50-6), Polygonum cuspidatum (Japanese knotweed extract) (e.g., CAS No: 501-36-0), Filipendula vulgaris (dropwort) (e.g., CAS No: 90028-75-4), Fraxinus rhynchophylla extract (e.g., CAS No: 8001-73-8), Prunus persica extract (e.g., CAS No: 84012-34-0), Rosa rugosa (rose) (e.g., CAS No: 92347-25-6), Meadowsweet Extract (e.g., CAS No: 84775-57-5), Pyrus malus Flower- and bark extracts (e.g., CAS No: 85251-63-4), Triticum Vulgare seed extract (e.g., CAS No: 84012-44-2), Hordeum Vulgare (barley) seed extract, Panax ginseng root extract (e.g., CAS No: 84650-12-4), Licorice Root Extract (e.g., CAS No: 84775-66-6), Blueberry (Bilberry) Fruit Extract (e.g., CAS No: 84082-34-8), Pterocarpus marsupium Bark Extract (CAS no: 84604-08-0), or any combination thereof. Pterocarpus marsupium Bark Extract was found to be particularly suitable and as such is a preferred ingredient for this application.

In the development of topical therapeutic compound for the abovementioned purposes it was found that higher concentrations of sugar- or sugar analogs (e.g. mannitol, dextrose, sucrose, glycerine) would frequently result in crystallization of the sugar compounds, which would make the composition unsuited for its purpose in that these tiny crystals, which would increase in concentration as the composition was exposed to air or the passage of time, would form an abrasive and highly uncomfortable texture against the skin (similar in feeling to beach sand or sand paper). Through experimentation it was discovered that a combination of sugars (e.g. mannitol with dextrose) with or without the addition of corn syrup (also known as glucose-fructose) would impede or completely eliminate the crystallization process, allowing for a higher concentration of these ingredients in solution. Furthermore, it was noted that this combination of sugars also had a surprising benefit of actually enhancing the primary anti-inflammatory, analgesic and therapeutic effects.

Furthermore, it was also discovered that using this method, the consistent crystallization of Mannitol, which would be expected to occur in cold environments, for example if an amount of topical compound was exposed to winter temperatures or a refrigerator, was significantly mitigated or eliminated.

An additional finding was that with a combination of sugars, the undesirable burning sensation on the skin caused by menthol (one potential therapeutic agent, which could be combined in the therapeutic compound) was significantly mitigated. Furthermore, the undesirable, strong smell of menthol was also mitigated with the inclusion of a terpene (e.g. D-Limonene), which in combination with the abovementioned sugar combination, drastically reduced the duration (how long the smell was noticeable to the user after application) and intensity of the menthol smell.

Based on the abovementioned developments, examples of therapeutic compounds derived from these findings are as follows:

Example topical composition 1:

| Component | Percentage of Total Volume |
|---|---|
| Water | 20-60% |
| One or more Stabilizers | 0.5-10% |
| One or more Emulsifiers | 3-10% |
| One or more Humectants | 5-20% |
| One or more Skin protectants | 0.5-10% |
| One or more Viscosity' controllers | 0.5-5% |
| One or more Permeation enhancers | 2-10% |
| One or more Preservative/antimicrobials | 1-10% |
| One or more Masking fragrances (optional ) | 0.25-1% |
| One or more Buffering agents | 0.25-5% |
| Primary therapeutic compound | 0.2-20% |
| CBD, anti-inflammatory) | (e.g. menthol, lidocaine, |
| | wintergreen |
| Primary sugar or sugar alcohol | 5.0-30% |
| | (e.g. dextrose, mannitol, |
| | glycerol) |
| Secondary sugar or sugar alcohol | 1.0-15% |
| | (e.g. dextrose, mannitol, |
| | glycerol) |

Example therapeutic topical composition 2.

| | |
|---|---|
| 35-58% | Distilled Water |
| 0.15-3% | Xanthan Gum |
| 1.8-3.0% | Vegetable Glycerin |
| 2.0-3.5% | Glycerin Stearate |
| 5.0-8.0% | Sunflower Oil |
| 2.5-4.0% | Caprylic Capric Trigylceride |
| 7.5-12% | Shea butter |
| 0.6-1.0% | Cerecin Wax |
| 1.8-3.0% | Lecithin |
| 1.2-2.0% | Aloe Vera |
| 1.9-3.0% | Disodium EDTA |
| 0.48-0.8% | Radish root |
| | (or form of antimicrobial agent) |
| 1.2-2.0% | sodium hyaluronate |
| 1.0-2.5% | D-Limonene |
| 0.6-1.0% | Optiphen |
| 0.5-3% | Zinc Oxide |
| 10-20% | Dextrose |
| 0.5-1.5% | L- Arginine |
| 0.1-5% | Menthol |
| 5.0-20.0% | Mannitol |
| 5.0-15% | MSM |

Example therapeutic topical composition 2

| | |
|---|---|
| 45-60% | Distilled Water |
| 0.1-0.4% | Xanthan Gum |
| 1-3.00% | Vegetable Glycerin |
| 1-3.50% | Glycerin Stearate |
| 3-8.00% | Sunflower Oil |
| 2-4.00% | Caprylic Capric Trigylceride |
| 5-15% | Shea butter |
| 0.2-3.00% | Cerecin Wax |
| 1-8.00% | Lecithin (did we up this?) |
| 0.5-3.00% | Aloe Vera |
| 1-3.00% | Disodium EDTA |
| 0.2-1.5% | Radish root |
| 1-3.00% | Sodium hyaluronate |
| 0.5-5.00% | D-Limonene |
| 0.5-2.00% | Arnica |
| 0.5-2.00% | Optiphen |
| 5-20.00% | Dextrose |
| 1-10.00% | Glucose-Fructose (corn syrup) |
| 0.1-1% | L-Arginine |
| 5-15.00% | MSM |
| 10-20% | Mannitol |
| 0.25-5% | Menthol |
| 0.25-1% | Pterocarpus Marsupium Extract |
| 0.5-8.00% | Vitamin D3 |

Example therapeutic topical composition 3:

| | |
|---|---|
| 45-60% | Distilled Water |
| 0.1-0.4% | Xanthan Gum |
| 1-3.00% | Vegetable Glycerin |
| 1-3.50% | Glycerin Stearate |
| 3-8.00% | Sunflower Oil |
| 2-4.00% | Caprylic Capric Trigylceride |
| 5-15% | Shea butter |
| 0.2-3.00% | Cerecin Wax |
| 1-8.00% | Lecithin (did we up this?) |
| 0.5-3.00% | Aloe Vera |
| 1-3.00% | Di sodium EDTA |
| 0.2-1.5% | Radish root (or form of antimicrobial agent) |
| 1-3.00% | Sodium hyaluronate |
| 0.5-5.00% | D-Limonene |
| 0.5-2.00% | Arnica |
| 0.5-2.00% | Optiphen |
| 5-20.00% | Dextrose |
| 1-10.00% | Glucose-Fructose (corn syrup) |
| 0.1-1% | L-Arginine |
| 5-15.00% | MSM |
| 10-20% | Mannitol |
| 0.25-5% | Menthol |
| 0.25-1% | Pterocarpus Marsupium Extract |

Example therapeutic topical composition 3:

| | |
|---|---|
| 45-60% | Distilled Water |
| 0.1-0.4% | Xanthan Gum |
| 1-3.00% | Vegetable Glycerin |
| 1-3.50% | Glycerin Stearate |
| 3-8.00% | Sunflower Oil |
| 2-4.00% | Caprylic Capric Trigylceride |
| 5-15% | Shea butter |
| 0.2-3.00% | Cerecin Wax |
| 1-8.00% | Lecithin (did we up this?) |
| 0.5-3.00% | Aloe Vera |
| 1-3.00% | Disodium EDTA |
| 0.2-1.5% | Radish root (or form of antimicrobial agent) |
| 1-3.00% | Sodium hyaluronate |
| 0.5-5.00% | D-Limonene |
| 0.5-2.00% | Optiphen |
| 5-20.00% | Dextrose |
| 1-10.00% | Glacose-Fructose (corn syrup) |
| 0.1-1% | L-Arginine |
| 5-15.00% | MSM |
| 10-20% | Mannitol |
| 0.25-1% | Pterocarpus Marsupium Extract |
| 0.5-8.00% | Vitamin D3 |

Example therapeutic topical composition 4:

| | |
|---|---|
| 45-60% | Distilled Water |
| 0.1-0.4% | Xanthan Gum |
| 1-3.00% | Vegetable Glycerin |
| 1-3.50% | Glycerin Stearate |
| 3-8.00% | Sunflower Oil |
| 2-4.00% | Caprylic Capric Trigylceride |
| 5-15% | Shea butter |
| 0.2-3.00% | Cerecin Wax |
| 1-8.00% | Lecithin (did we up this?) |
| 0.5-3.00% | Aloe Vera |
| 1-3.00% | Disodium EDTA |
| 0.2-1.5% | Radish root (or form of antimicrobial agent) |
| 1-3.00% | Sodium hyaluronate |
| 0.5-5.00% | D-Limonene |
| 0.5-2.00% | Optiphen |
| 5-20.00% | Dextrose |
| 0.1-5% | L-Arginine (or Sodium Hydroxide) |
| 10-20% | Mannitol |
| 0.25-1% | Pterocarpus Marsupium Extract |
| 0.1-8% | Sodium Bicarbonate |

Example therapeutic topical composition 5

| | |
|---|---|
| 45-60% | Distilled Water |
| 0.1-0.4% | Xanthan Gum |
| 1-3.00% | Vegetable Glycerin |
| 1-3.50% | Glycerin Stearate |
| 3-8.00% | Sunflower Oil |
| 2-4.00% | Caprylic Capric Trigylceride |
| 5-15% | Shea butter |
| 0.2-3.00% | Cerecin Wax |
| 1-8.00% | Lecithin (did we up this?) |
| 0.5-3.00% | Aloe Vera |
| 1-3.00% | Disodium EDTA |
| 0.2-1.5% | Radish root (or form of antimicrobial agent) |
| 1-3.00% | Sodium hyaluronate |
| 0.5-5.00% | D-Limonene |
| 0.5-2.00% | Optiphen |
| 0.1-1% | L-Arginine |
| 10-30% | Mannitol |
| 3-10.0% | Glucose-Fructose (corn syrup) |
| 0.25-1% | Pterocarpus Marsupium Extract |

Example therapeutic topical composition 6:

| | |
|---|---|
| 45-60% | Distilled Water |
| 0.1-0.4% | Xanthan Gum |
| 1-3.00% | Vegetable Glycerin |
| 1-3.50% | Glycerin Stearate |
| 3-8.00% | Sunflower Oil |
| 2-4.00% | Caprylic Capric Trigylceride |
| 5-15% | Shea butter |
| 0.2-3.00% | Cerecin Wax |
| 1-8.00% | Lecithin (did we up this?) |
| 0.5-3.00% | Aloe Vera |
| 1-3.00% | Disodium EDTA |
| 0.2-1.5% | Radish root (or form of antimicrobial agent) |
| 1-3.00% | Sodium hyaluronate |
| 0.5-5.00% | D-Limonene |
| 0.5-2.00% | Optiphen |
| 0.1-1% | L-Arginine |
| 10-30% | Mannitol |
| 3-10.0% | Glucose-Fructose (corn syrup) |
| 0.25-1% | Pterocarpus Marsupium Extract |
| 0.1-2% | Sodium Bicarbonate |

Example therapeutic topical composition 7:

| | |
|---|---|
| 45-65% | Distilled Water |
| 0.1-0.2% | Xanthan Gum |
| 1-5% | Vegetable Glycerin |
| 2-5% | Glycerin Stearate |
| 6-10% | Sunflower Oil |
| 2-6% | Caprylic Capric Trigylceride |
| 8-15% | Shea butter |
| 0.5-2% | Cerecin Wax |
| 2-6% | Lecithin |
| 1.5-4% | Aloe Vera |
| 2-5% | Disodium EDTA |
| 0.5-1.0% | Radish root |
| 1.0-3.0% | Sodium hyaluronate |
| 1.0-10.0% | Terpene (e.g. D-Limonene) |
| 0.5-2.0% | Optiphen |
| 3-10.0% | Glucose-Fructose (corn syrup) |
| 10-30.0% | Dextrose |
| 0.2-0.5% | L-Arginine |
| 10-20.0% | Mannitol |
| 4.00% | Lidocaine |

Example therapeutic topical composition 8

| | |
|---|---|
| 45-65% | Distilled Water |
| 0.1-0.2% | Xanthan Gum |
| 1-5% | Vegetable Glycerin |
| 2-5% | Glycerin Stearate |
| 6-10% | Sunflower Oil |
| 2-6% | Caprylic Capric Trigylceride |
| 8-15% | Shea butter |
| 0.5-2% | Cerecin Wax |
| 2-6% | Lecithin |
| 1.5-4% | Aloe Vera |
| 2-5% | Disodium EDTA |
| 0.5-1.0% | Radish root |
| 1.0-3.0% | Sodium hyaluronate |
| 1.0-10.0% | Terpene (e.g. D-Limonene) |
| 0.5-2.0% | Optiphen |
| 3-10.0% | Glucose-Fructose (corn syrup) |
| 10-30.0% | Dextrose |
| 0.2-0.5% | L-Arginine |
| 10-20.0% | Mannitol |

Example therapeutic topical composition 9:

| | |
|---|---|
| 45-65% | Distilled Water |
| 0.1-0.2% | Xanthan or Guar Gum |
| 1-5% | Vegetable Glycerin |
| 2-5% | Glycerin Stearate |
| 6-10% | Sunflower Oil |
| 2-6% | Caprylic Capric Trigylceride |
| 8-15% | Shea butter |
| 2-6% | Lecithin |
| 1.5-4% | Aloe Vera |
| 2-5% | Disodium EDTA |
| 1.0-3.0% | Sodium hyaluronate |
| 1.0-10.0% | Terpene (e.g. D-Limonene) |
| 0.5-2.0% | Optiphen |
| 10-30.0% | Dextrose |
| 0.2-1.0% | Sodium Hydroxide |
| 0.1-8.0% | Sodium Bicarbonate |
| 10-30.0% | Mannitol |
| 4.00% | Lidocaine |

In each of the abovementioned embodiments, water might be replaced by Glycerin (Glycerol), in order to eliminate reactivity associated with water, sodium bicarbonate and dextrose. This is a solution that was found to be a highly effective stabilizing measure for stabilizing the compound and prolonging shelf-life.

Furthermore, it was found that combining two forms of sugar (e.g. dextrose and mannitol) served to further stabilize the formula, whilst synergistically improving the clinical effect of the therapeutic compound.

Now referring to compound embodiment #9, note that Lidocaine might be replaced by any agent known to have analgesic properties. Furthermore, Dextrose might be discarded should this be indicated in a particular therapeutic context.

Method for Treatment of Body Symptoms and Pain by Adhesive Therapeutic Interface

A systematic approach involving device systems disclosed herein can involve the following steps, which can be performed in sequence or individually depending on the particular case presentation:

Step 1: one or more symptom-generating nerves can be identified (e.g., localization of these nerves may be achieved by one or more of palpation; negative or positive pressure on overlying tissue; visual scanning for diagnostic evidence, movement of limbs so as to impart compression or traction; anatomical understanding on the part of the trained clinician who may recognize the symptomatic area as being associated with a particular nerve or combination of nerves). Isolation of these target tissues can occur before use in a prescriptive fashion and might not be a component of every application session.

Step 2: a portion of adhesive interface is applied to the skin overlying the target nerve or nerves to form a contact between the interface and the skin (the underlying skin may optionally be prepared by manual deformation such that the adhesive interface will maintain a form or tension to the tissues not otherwise maintainable without the support offered by the interface. Examples of materials that can form this interface include, bandage, plaster, firm- or elastic-forms of tape, and wax. The application of the interface may also create a smooth or irregular surface to produce anchor points along the outermost surface to allow for contact with the hand or some other form of device). Adhesion with the superficial surfaces can occur in a series of applications separated by periods of time in order that the user might experience movement at each of these periods of time with body tissue tensioned in different configurations.

Step 3: making contact with the outer surface of the interface component, the clinician or user can impart positive (pressure or pressing) or negative (traction or pulling) forces on the interface in multiple potential vectors or directions, which will secondarily therapeutically manipulate the underlying tissue (with the intention of mechanically liberating symptom-causing nerves or other target tissues). This step can be augmented by passive or active movement of one or more limbs (especially the limb underlying the interface) with the intention of further mobilizing the tissue underlying the interface.

Step 4: The adhering interface can be removed, which can debride or remove the superficial skin or tissue layer(s), which can serve multiple potential benefits (including subsequent facilitation of passage of therapeutic topical agents and removal of damaged, foreign or non-viable tissues from the surface).

Step 5: with the superficial and deeper tissues now prepared, a therapeutic compound is applied over the target area, which may be in the form of any one or combination of topical therapeutic types (e.g. lotion, gel or balm), such as a therapeutic compound on an interface (e.g. tape, dressing or patch), therapeutic vapor or gas, therapeutic light source (e.g. cold laser), therapeutic oscillatory therapy (e.g. vibration, percussion, shockwave) therapeutic thermal source (e.g. cryotherapy or heat). This step can be augmented with movement of tissue or limb.

Formulation Method for Improving Efficacy of Topical Therapeutic Agents

A number of topical analgesics can be used for those suffering from pain and symptoms of bodily injury Some of these agents include Menthol, Camphor, Phenolic compounds, Polyphenols, Salicylates, Cannabinoid, Wintergreen, Trolamine salicylate, Allantoin, Aluminium acetate, Trolamine, Zinc Oxide, Zinc Acetate, Juniper Tar, Butambem Picrate, Lidocaine, Benzocaine. Hydrocortisone. Hydrocortisone acetate, and topical opioids. While these agents have demonstrated some positive analgesic and in some cases, anti-inflammatory capacities, there is still a great need for improvement in overall efficacy of each of these, as well as other topical analgesic agents.

The current disclosure describes a system for improving the efficacy of topical analgesics by promoting metabolism of local tissues, including target tissues of the therapeutic agent. This compounding method promotes the cellular uptake of the therapeutic agent and consequently, the therapeutic response of the target tissue. Target tissue in this instance could mean superficial nerves, muscle, tendon, tissues of the skin, scalp, eye, nasal and oral structures, or internal structures of the body. This said, superficial nerves can derive particular benefit to this feature due to their capacity to generate pain and problems with tissue integrity. For this reason, a particular method is mentioned herein to address neurogenic involvement in various conditions.

In one embodiment of the method, one or more therapeutic- or analgesic agents is presented in a stable medium or compound comprising of an amount of sugar and/or sugar alcohol, such as dextrose and/or mannitol, and some form of buffering agent, the latter serving to raise the pH of the medium to a state of relatively alkalinity. An example alkalinity would be between pH 8-9. The medium or compound can include an excipient, which can include an amount of water, an amount of one or more emollients, an amount of one or more emulsifiers, an amount of one or more penetration enhancers (e.g. hyaluronidase, polysaccharide, lecithin or lipid complex), an amount of one or more humectants, and an amount of one or more preservatives or antimicrobial agents. The aforementioned combination of agents, or subsets thereof, will sometimes be referred to herein as ‘vehicle’.

In certain instances the vehicle might include or be supplemented by any one or combination of perforated or un-perforated occlusive dressing or patch (sometimes described herein as a ‘therapeutic interface’), lipophilic material (e.g. paraffin, oil, fat, wax, fatty acid, alcohol, esther or silicone), anhydrous lipid (e.g used as absorption or emulsifying base), water-oil emulsion, powder (e.g. clay, organic or inorganic), oil-water emulsion (e.g. aqueous cream).

The application of buffered sugar or sugar alcohol medium can exert an analgesic effect on superficial nerves. Through experimentation, it has been found that this medium also appeared to improve the efficacy of other therapeutic compounds, which in some cases were not directed or developed to affect neurogenic inflammation, especially topical analgesics. Without being bound by theory, this “buffered sugar” medium is believed to exert a positive benefit on mitochondrial function, which imparts a secondary benefit on general cellular metabolism, which can positively influence the uptake of the primary therapeutic agent. A base compound is thus reported with which to deliver and improve a variety of topical therapies. Some of the benefits observed are greater overall efficacy derived from the primary agent (primary therapeutic agents generally work better, including in most cases, prolonged therapeutic benefit and more rapid time to onset of therapeutic effect); less of the primary agent required to achieve a therapeutic outcome (this is particularly valuable for therapeutic agents that carry higher risk or higher frequency of adverse side effects); lower volume of topical compound required to achieve therapeutic effect (a great convenience to the user who is consequently required to rub less of a topical agent into the skin with each application, saving time and cost).

The sugar can be a natural or artificial compound, which can include glucose, dextrose, sucrose, fructose, etc. In some situations, dextrose has been found to be the preferred agent, but other sugars can also be used. An example range of concentration of sugar (e.g., dextrose) is about 5-45% by weight. The sugar alcohol can include mannitol, glycerol, sorbitol, xylitol, lactitol, isomalt, maltitol and/or hydrogenated starch hydrolysate, or combinations thereof. In some situations, mannitol and glycerol may be preferred, although other sugar alcohols can be used. An example range of concentration of sugar alcohol (e.g., mannitol or glycerol) is about 5-45% by weight. Buffer or alkalizing agents can include any one or combination of sodium bicarbonate, L-Arginine, sodium citrate, alpha hydroxyl acid, glycolic acid, triethanolamine, diethanolamine, ethanolamine, sodium phosphate, calcium carbonate, potassium carbonate, magnesium carbonate, and citric acid).

One embodiment of the compound can include an amount of menthol or camphor, ranging from about 0.5-20% by weight; an amount of sugar or sugar alcohol, ranging from about 5-40% by weight; and an amount of vehicle.

One embodiment of the compound can include an amount of Salicylate (e g. Wintergreen or Trolamine salicylate), ranging from about 0.1-25% by weight, an amount of sugar or sugar alcohol, ranging from about 5-40% by weight; and an amount of vehicle In a certain therapeutic application, this compound may be included in the aforementioned therapeutic interface as the interface therapeutic compound.

One embodiment of the compound can include an amount of hemp-, Cannabis-derivative or cannabinoid, ranging from about 0.1-60% of total volume or weight, or about 2 mg-2000 mg per application: an amount of sugar or sugar alcohol, ranging from about 5-40% by weight or volume, and an amount of vehicle In a certain therapeutic application, this compound (as well as the various other compound embodiments disclosed herein) may be included in the aforementioned therapeutic interface as the interface therapeutic compound.

One embodiment of the compound can include an amount of TOR- or mTOR-modulator or Polyphenol (e.g. Resveratrol, Quercetin, Pterostilbene, Eugenol, Flavone (including for example, Ginkgo biloba, luteolin, tangeritin, and sinensetin), Isoflavonoid (including for example, genistein, and daidzein), Flavanones (including for example, hesperitin, narngenin, eriodictyol), Flavonol (including for example, quercetin, myricetin, fisetin), Neoflavonoid, Flavanonol (e.g., Catechin), Olligostillbenoid, stilbene, or flavonoid), ranging from about 0.1-30% by weight or volume, an amount of sugar or sugar alcohol, ranging from about 5-40% by weight or volume; and an amount of vehicle. In a certain therapeutic application, this compound may be included in the aforementioned therapeutic interface as the interface therapeutic compound.

In an alternative embodiment to the immediately above, the TOR- or mTOR-modulator or Polyphenol can be supplemented or replaced with a fruit or seed extract known to include these molecules. The discovered advantage of this group of ingredients is that they are generally more stable in solution than isolated polyphenols, and in some instances, better tolerated by users. Examples include, Adaptogen or adaptogen extract (e.g. Rhodiola rosea Extract (e.g., CAS No: 97404-52-9), Kakadu Plum Extract (e.g., CAS No: 56-81-5, 7732-18-5, 1176234-54-0, 122-99-6), Acai Fruit Extract (e.g., CAS No: 879496-95-4), Salvia miltiorrhiza Bunge (danshen) (e.g., CAS No: 90106-50-6), Polygonum cuspidatum (Japanese knotweed extract) (e.g., CAS No: 501-36-0), Filipendula vulgaris (dropwort) (e.g., CAS No: 90028-75-4), Fraxinus rhynchophylla extract (e.g, CAS No: 8001-73-8), Prunus persica extract (e.g., CAS No: 84012-34-0), Rosa rugosa (rose) (e.g., CAS No: 92347-25-6), Meadowsweet Extract (e.g, CAS No: 84775-57-5), Pyrus malus Flower- and bark extracts (e.g., CAS No: 85251-63-4), Triticum vulgare seed extract (e.g., CAS No: 84012-44-2), Hordeum vulgare (barley) seed extract, Panax Ginseng root extract (e.g., CAS No: 84650-12-4), Licorice Root Extract (e.g., CAS No: 84775-66-6), Blueberry (Bilberry) Fruit Extract (e.g., CAS No: 84082-34-8).

An example embodiment can include an amount in the range of about 0.25-15% by weight or volume of any combination of the following: Blueberry Fruit Extract (e.g., CAS No: 84082-34-8), Acai Fruit Extract (e.g, CAS No: 879496-95-4). Plum Extract (e.g., CAS No: 56-81-5, 7732-18-5, 1176234-54-0, 122-99-6), Bilberry Extract (e.g., CAS No: 879496-95-4), Pterocarpus marsupium (kino bark) extract (e.g., CAS No: 84604-08-0).

A method is described herein for using fruit and plant extracts in a particular technique embodiment directed at symptomatic and/or painful nerves and nerve fibers.

One embodiment of the compound can include an amount of therapeutic peptide or peptide fragment, e.g., which can be categorized under “regenerative agents” in this application (e.g. any one of or combination of AOD9604 (AOD9604+hyaluronic acid (HA)), Amlexanox, Ammonium Tetrathiomolybdate, Aniracetam, AOD 9604 modified form (peptide fragments) of amino acids 176-191 of GH polypeptide, Pentadecapeptide BPC 157 (partial sequence of body protection compound (BPC), fragments of cerebrolysin, Follistatin, fragments of growth hormone molecule, Pentosan polysulfate (oversulphated glycosaminoglycan), Melanocortins, Thymosin, Vasoactive Intestinal Polypeptide (VIP), Thymulin), such as ranging from about 0.1-50% by weight or volume; an amount of sugar or sugar alcohol, ranging from about 5-40% by weight or volume; an amount of vehicle. In a certain therapeutic application, this compound may be included in the aforementioned therapeutic interface as the interface therapeutic compound.

One embodiment of the compound can include an amount of Platelet-rich plasma (PRP) or other autologous substance, such as obtained from the user of the embodiment (these agents can be classed under “regenerative agents” in this disclosure), such as ranging from about 0.1-70% by weight or volume: an amount of sugar or sugar alcohol, ranging from about 5-40% by weight or volume; and an amount of vehicle In a certain therapeutic application, this compound may be included in the aforementioned therapeutic interface as the interface therapeutic compound. This embodiment can be of particular value as a therapeutic application as PRP therapy is intended to create tissue turnover in the target area. This regenerative effect could be facilitated by enhanced mitochondrial and metabolic function.

The inventor has produced a series of embodiments of the compound with the intention of meeting the unmet need for a therapeutic compound, formulated for high efficiency through the use of ingredients with dual-functions (e.g, allowing for augmentation of therapeutic benefit with reduced overall cost and lower total volume of compound with fewer ingredients) in the context of the primary objective of delivering a topical compound with improved analgesic benefit without exposing the body to harmful or contentious compounds as well as avoiding the pitfall of using substantial comedogenic (pore-clogging, pimple- or blackhead-causing) ingredients.

One such embodiment includes one or more sugars or sugar alcohols or combination thereof (e.g., comprising between about 4-40% of total volume or weight); an amount of alkalizing agent or buffer (e.g., comprising between about 1-10% of total volume or weight), an amount of emollient (e.g., including a humectant), a amount of a penetration enhancer and an amount of a preservative/antimicrobial agent. The pH of this compound can be about 7.6-10. In a certain instance, the embodiment can be devoid of a buffer or alkalizing agent where this effect is not selected, e.g., based on certain factors associated with the user case or compounding approach. In this embodiment, the emollient can include one or more of glycerol, caprylic capric triglyceride, c30-45 alkyl methicone, c30-45 alkyl olefin, microcrystaline wax, polyethylene (or other synthetic wax), silica dimethyl silylate, sodium PCA, xanthan Gum, sodium hyaluronate, ceresin wax. Penetration enhancer can include one or more of vegetable oil (e.g. argan oil; jojoba oil; shea butter), terpene (e.g. d-Limonene, a-pinene, limene), tetrasodium EDTA, Cold-pressed Aloe, and Arnica. The preservative and/or antimicrobial agent can include any one or combination of Naticide, Radish Root, Phenoserve, Optiphen. This embodiment can be modified for the purpose of improving perceived muscular function, reducing post-exertional discomfort or improving physical performance, whereas such an application can use lower quantities of certain ingredients, such as sugar or sugar alcohol where such an agent might promote undesirable sensation of the skin (potentially including tackiness or stickiness), in which case the embodiment would comprise of an amount of sugar or sugar alcohol at about 1-10% of the total volume or weight. Furthermore, in the latter case, an additional measure might be implemented to improve the applicability of the compound for sports, such as increased humectant to provide a sustained release effect whereas the continual attraction of fluid to the skin surface will serve to promote gradual release of active ingredient residue on the skin, as well as reducing perception of stickiness or tackiness.

Yet another embodiment includes an analgesic or tissue regenerative agent at about 0.1-40% of volume or by weight (e.g., including one or more of Menthol, Camphor, Phenol or Phenolic compound, Polyphenol, Salicylate, Allantoin, Aluminium acetate. Trolamine, Zinc Oxide, Zinc Acetate, Juniper Tar, Butambem Picrate, Lidocaine, Benzocaine, Hydrocortisone, Hydrocortisone acetate, Opioid, PRP, Autologous compound, Peptide or Peptide fragment), one or more sugars or sugar alcohols or combination thereof (comprising between about 4-40% of total volume or weight); an amount of alkalizing agent or buffer (comprising between about 1-10% of total volume or weight), an amount of emollient (e.g., including a humectant), an amount of a penetration enhancer and an amount of a preservative/antimicrobial agent. The pH of this compound can be about 7.6-10. In a certain instance, the embodiment can be devoid of a buffer or alkalizing agent where this effect is not selected, based on certain factors associated with the user case or compounding approach. In this embodiment, the emollient can include one or more of glycerol, caprylic capric triglyceride, c30-45 alkyl methicone, c30-45 alkyl olefin, microcrystaline wax, polyethylene (or other type of synthetic wax), silica dimethyl silylate, sodium PCA, xanthan Gum, sodium hyaluronate, ceresin wax. Penetration enhancer can include one or more of vegetable oil (e.g. argan oil; jojoba oil; shea butter), terpene (e.g. d-Limonene, a-pinene, limene), tetrasodium EDTA, Cold-pressed Aloe, and Arnica. The preservative and/or antimicrobial agent can include any one or combination of Naticide, Radish Root, Phenoserve, Optiphen.

Therapeutic System for Simultaneously Stimulating Motor, Sensory and Analgesic Systems of the Nervous System and Method of Use

Various forms of electrical stimulation have been applied to the human body as a therapy. Examples of these include Transcutaneous electrical nerve stimulation (TENS): a form of transcutaneous electrical stimulation of sensory nerves and receptors for transient analgesic effect: Electrical muscle stimulation (also known as EMS, neuromuscular electrical stimulation, or electromyostimulation): a form of transcutaneous stimulation that results in a contraction of muscle, which is typically used to gain strength in muscle Other forms of electrical stimulation of the human body include Galvanic Stimulation: a form of transcutaneous stimulation that uses direct current to have a variety of potential effects, including reducing edema (when applied to injury sites), stimulating the vestibular system (when directed at the inner ear) and generally stimulating sensory nerves In some instances, it can be beneficial to avoid or to reduce what is termed “passive therapies”, that is therapies that do not require active engagement from the user (who is presumed to be suffering from symptoms or some form of adverse condition). The belief is that thus type of therapeutic approach promotes avoidance, passive coping, a trend toward relief-seeking without improvements in perceived self-efficacy and general promotion of disability. The alternative to this approach would be to employ therapies that promote active engagement on the part of the patient, such as physical exercises that bring about relief over time and promote a more active approach to self-care rather than a need to physically disengage and passively receive therapy. A further criticism of passive motor system therapies is that they are less efficient at promoting gains in strength, motor and muscle capacity than active exercise.

What follows is a description of a device and therapeutic method that utilizes transcutaneous energy to impart multiple simultaneous benefits and can be applied to treat a number of symptoms and disorders as well as improve human function, whilst promoting active engagement on the part of the user. Furthermore, whilst this system dominantly utilizes a form of muscle or motor stimulation, there is a surprising result in that it markedly inhibits pain and stimulates the sensory system. An additional unexpected benefit of this system is that it appears to rapidly and positively bring about gains in motor function and strength not typically seen in passive motor therapies.

Now to a description of the component parts of the system. In broad terms the system can include a controller component, a sensor component and a processor or integrating component. The system controller portion may be a voltage or current source designed to generate electrical signals that are bound for the body of the user. This system can alter pulse width and intensity, among other physical parameters of electrical output. The sensor system can receive signals from one or more sensors attached to—, actuated by— or placed adjacent to—the users body such that activities of the user trigger such a sensor, which will in turn result in electrical signals being delivered to the sensor portion.

This controller portion of the system can be accessible by one or more outputs. For example, one or more leads can be connected to these outputs by removable jacks. One or more of these leads are attached to one or more transcutaneous electrodes. There can be input ports to the device, which accept leads that are connected to a sensor component of the system, which can take various forms In one embodiment, the sensor could take the form of a mechanism that generates signal current when it is deformed or stretched. As such, it could act as an embedded or attached component to some form of resistance band or cable, such that when the resistance band or cable is elongated, stretched or deformed, an electrical signal current is generated that is transmitted via cable (or cable-free technology such as Bluetooth™) to the system inputs).

In one embodiment of this system, the output of the muscle stimulating system is around 20-80-volts with a frequency of around 40-120-Hz.

In some embodiments, there is first a component formed of deformable material fashioned into a continuous loop, which may be constructed out of rubber, latex, nylon, non-latex elastic or similar material that allows for reversible deformation (suitable examples of such a component include Theraband® resistance loops). The width of this band can be about 2-4 inches, while the length (e.g., circumference of the closed loop) can be about 22-27 inches, however, the width and length may be of any length or width deemed appropriate to impart the appropriate fit and length, tension relationship for the individual user. In an example embodiment, the loop is covered by a softer, compliant material, such as spandex, lycra, stretch satin, stretch velvet, polyester, nylon spandex or some form of rapid-drying stretch fabric. The purpose of the outer covering is for comfort and to prevent pinching of limb hair or skin. This band (and its outer covering) can be stretched by a user's effort. In this embodiment, the user can place the band around the thighs or lower legs but it could be placed elsewhere on the body. In this placement, the user might perform some sort of activity to stretch the band, such as but not limited to actual exercises or movements such as a clamshell (e.g., side lying- or supine-abduction with external rotation of the hips and thighs), monster-walk (e.g., walking with accentuated space between the lower limbs so as to accentuate activity of the hip abductors), squat or fire-hydrant (e.g., extension of the hip in the quadruped position) or similar activities intended to emphasize muscles whose primary activity is resisted by the band. Examples of such muscles would include one or more muscles of the gluteal complex (e.g. gluteus-maximus, -medius, -minimus), or one or more shoulder girdle muscle (e.g. serratus anterior).

A second component of this example embodiment is a displacement- or stretch sensor, which is attached to the aforementioned band such that as the band component is elongated, the stretch sensor is activated and the degree of stretch is transposed into a corresponding electrical signal In another embodiment, stretch may be replaced with proximity such that the system contains two or more proximity sensors or system components that create signal and/or transduce information as they are moved toward or away from either other, thereby acting as a sensory of proximity with an intended effect that is similar to that of the stretch sensor.

Returning to the stretch sensor embodiment, when the band is stretched, the sensor transmits signals to the sensor portion of the device system. In some embodiments, this is via a Bluetooth™ transmitter, or other wireless communication device, which transmits to a receiver within the sensor portion of the device. In another embodiment, this might be via simple wires that are coupled via input ports on the device housing to the sensor portion of the device. The processing portion of the system can receive this input and per pre-defined programmed parameters and logic, can dynamically respond (e.g., with moment-to-moment processing to direct varying responses) to the received information by varying the output from the controller component. The system output, in this example embodiment, would be increasing or decreasing certain parameters of electrical signals directed to transcutaneous electrodes placed on the user's body. These parameters might include but are not limited to pulse width and stimulation intensity, which can elicit a desired effect in target sensory nerves- or muscle tissue. In an embodiment wherein the deformable loop is placed on the thighs or lower limbs, the transcutaneous electrodes might be placed over the gluteal muscles (gluteus maximus, -minimus, or -medius). In such a configuration, as the user stretches the band they will be necessarily engaging the gluteals and as they do so, the stretch sensor will generate a barrage of continuous signals, which the processing portion will integrate and direct a response from the controller portion to increase electrical stimulation of the same gluteals so as to further facilitate muscle contraction. Thus, in this configuration, the system will work to supplement the user's effort in a synergistic manner.

When a relatively weaker or debilitated user first engages the system, they can utilize a band with relatively lower resistance and also depend on relatively more stimulus from the output portion of the device. As they become stronger, they can utilize a band with more resistance and be able to recruit more of their natural strength with a relatively diminished reliance on the electrical output. This said, there may be a desire, in some cases, to continually ramp up the electrical output to target muscles in order to continually provide maximal tolerable stimulus to the target muscle Once again, the deformable band could be replaced by two or more sensors that detect distance between said sensors such that as a particular joint or joint complex is opened, the sensors would move further apart and as such transduce this moment-to-moment increase in distance into a particular signal input into the system, which can elicit a corresponding output response. The system can be capable of transposing various stages of joint angulation into a particular electrical signal, which is integrated into the device system, which in turn outputs a particular response, which in this embodiment, is an electrical signal, which serves to stimulate target muscles.

In some embodiments, the device processor could invert its response to an incoming signal resulting from activation of the displacement or stretch sensor such that with no or relatively less displacement or stretch, the controller portion can emit signal to the user to activate the target muscle to a relatively greater extent, such that the user will have to overcome their own muscle contraction to displace or stretch the deformable material of the loop or strip, depending on the particular embodiment or application. Put another way, a greater amount of electrically induced muscle contraction will be experienced at the start of a movement, which will dissipate as the user moves away from the starting point, then again increase as the user re-approaches the starting point of the exercise cycle. This particular function can be a beneficial treatment method for movement disorders and disorders of tone (e.g., including Parkinson's disease, bradykinesia, dystonia, spinal cord injury, traumatic brain injury, aging and stroke), due to the fact that it emphasizes eccentric muscle function, which would emphasize brain systems typically affected by these disorders. The outcome in these cases would be improvements in gait, lowered risk for accidental fall, and less “freezing” events. This function, along with the primary function described above can be an excellent therapy for fall prevention in the elderly as age has been shown to disproportionately adversely affect motor systems of the brain associated with eccentric muscle function. This device and particular method is a very safe but rigorous form of exercise that can strengthen postural stabilizers of the hip and pelvis, whilst simultaneously driving positive plasticity in motor systems of the brain.

With respect to the abovementioned method for treating disorders and symptoms of the brain and central nervous system, this method and application, in one embodiment can be enhanced by certain audio-, visual-, tactile-, or other sensory stimuli, which can be directed by an interaction between the processor portion of the device system and a separate, mobile processing device such a custom device, mobile phone, tablet, laptop, computer or any other device capable of receiving, processing, and responding to information. In this embodiment, information from the processor component of the device is transmitted by any form of solid connection (e.g. wire or fiber optic) or remote transmission technology (e.g. radio, Bluetooth, infrared). Some of the potential audio-, visual- or tactile-stimuli delivered by the remote device could include functions or instructed tasks pertaining to timing (e.g. the user can be instructed to count without verbalization or to perform a task to a specific rate or rhythm to an external or internal cue); visualization (e.g. the user could be instructed, coached or guided through particular exercises involving visualization of certain concepts, which may or may not involve the body, memory, emotions, or autobiographical concepts or meditations or hypnosis); instructions on how to perform exercises or direction through a particular routine using the device system; visual exercises (e.g., potentially including ocular tracking, convergence, pursuit, saccade, antisaccade, virtual- or enhanced-reality, or any other therapeutic oculomotor or visual exercise that might enhance the directed therapy), therapeutic sounds (e.g. natural sounds, binaural beats, or any form of audio therapy); or any combinations thereof.

Now returning to the benefits of the some of the embodiments described herein. The abovementioned effect (e.g., the introduction of electrical stimulation as the user is exerting themselves against a specific external source of resistance) has multiple benefits, which are particularly valuable in rehabilitation, especially rehabilitation from pain and injury. Firstly, the system acts as a “spotter” or assistant by providing an assisting force to contract the active muscles. This is of particular benefit in deconditioned, bed-ridden or injured individuals who have difficulty recruiting the target muscle group. This has been found to be particularly true of hip, spinal and pelvic muscles, as well as deep muscles of the shoulder and scapulothoracic girdle Secondly, the electrical stimulus can be intentionally configured so as to be noxious to the user. For example, the user can be instructed to note when the stimulus becomes uncomfortable but not painful. The simplest parameter to modify to achieve a noxious stimulus is the stimulus intensity. The signal can be noxious (e.g., at least uncomfortable or even painful) in nature (although it need not be so to be effective), thereby imparting a simultaneous muscle-activating effect as well as an analgesic effect via a conditioned pain modulation (CPM) model Conditioned pain modulation is a method of recruiting endogenous analgesic systems of the central nervous system and brain by presenting a noxious sensory stimulus. Some embodiments disclosed herein provide both muscle recruitment as well as analgesia by noxious stimulus. This dual effect is particularly effective and useful in lower back pain.

In a certain embodiment of this system and method of treatment, stimulating electrodes can be placed on the plantar, lateral and/or medial surface of the feet, overlying the small instrinsic muscles of each foot. These electrodes can be directed to a single foot or both feet. In an example of therapeutic use of this system, an electrical discharge is released so as to forcibly contract the muscles of the feet, which may or may not be accompanied by an intentional or incidental noxious sensation in this target region. The user is then instructed to extend the toes and foot so as to activate muscle that would oppose the action induced by the activated electrodes, thereby overcoming the force of the contracted muscle to elongate the muscles targeted by the electrical stimulus. The user would then be instructed to relax the opposing muscles to allow the electrically-stimulated muscles to dominate again—the cycle can then be repeated in some cases. The system can then be directed to increase the electrical stimulus so as to more forceably contract the muscles as the user is directed to repeat one or more cycles of opposing muscle action to overcome the forceable contraction of the stimulated muscles. Again, this cycle can be repeated wherein the electrical stimulus intensity is incrementally increased as the user is able to overcome the resistence of the electrically-contracted muscle groups.

This activity can be coupled to the stimulation of the gluteal muscles such that in one embodiment of this treatment method, the user can be directed to simultaneously contract the gluteals whilst engaging the opposing muscles to the intrinsic flexors of the toes and foot In another embodiment, the stimulation of the feet might be an isolated activity, performed in absence of stimulation elsewhere in the body.

The benefit of this form of stimulation is that the user will benefit from three, simultaneous effects. 1) Activation and subsequent stabilization of muscles associated with normal posture and gait, thereby therapeutically off-loading multiple joints and tissues in the lower kinetic chain, including plantar muscles and plantar fascia, joints of feet, ankles, knees, hips and spine, cartilage along this chain and tendons (including achilles tendon) 2) Powerful stimulation of the central nervous system, especially the spinal cord and other sensory systems, including those associated with pain and sensory perception. This is expected to be particularly useful in improving sensory perception in peripheral polyneuropathy and other painful conditions of the body (especially lower limb and lower back conditions). The unique finding associated with this system is that contrary to popular belief in the field, this approach produces profound changes in sensory function as a consequence of activation of the be particularly useful in improving sensory perception in peripheral polyneuropathy and other painful conditions of the body (especially lower limb and lower back conditions). The unique finding associated with this system is that contrary to popular belief in the field, this approach produces profound changes in sensory function as a consequence of activation of the motor system. Typically, the sensory system is addressed with sensory stimuli. 3) Synergistically enhance the effect of therapeutic topicals described in this document by creating changes in the central nervous system to affect sensory thresholds centrally, while the topical therapeutics positively affect affected peripheral nerves.

A Treatment System and Treatment Method for Lower Back Pain, Conditions of the Proximal Joints, Lower Limbs and Balance and Locomotor Systems

As an extension and example embodiment of the abovementioned device system setup, an explanation is now directed to a treatment method for lower back pain and disorders of the spine. Throughout history and indeed to present day, treatments and therapies for lower back pain have uniformly been directed to the spine itself. This approach directs the therapy to the pelvis (which for purposes of explaining this device system and therapy method, might be thought of as the functional and structural foundation for the spine), in a manner as a highly effective and valuable treatment option for one of the most disabling and costly conditions in the world.

As a descriptive review of the abovementioned device system, utilized in this approach to the treatment of lower back pain, the system can include a form of resistance band or material that is intended to wrap around the lower limbs. The site for application can be at—, just above—or just below the knees in order to resist abduction, extension, external rotation and flexion of the femurs (e.g., actions initiated at the hip joint) but the band could be placed anywhere along the lower limbs so as to target desired muscles. As in the abovementioned description, this resistance band can, in certain embodiments, be supplemented or replaced by movement- or proximity sensors, which produce a response or electrical potential to transduce relative displacement, relative distance between sensors or movement of joints into a certain signal. This resistance and movement-sensing portion of the system is then connected to a controller portion, which could be by wire or wirelessly (e.g. Bluetooth or similar transmitting technology). The controller portion includes at least a processing component that receives, processes and produces responses to input received via the sensor associated with the resistance portion (e.g., portion tethered to the lower limbs). The controller portion may in a certain embodiment interact with an additional processing technology such as a mobile device (e.g. mobile phone, pad, or laptop) to translate the various parameters of the device into a graphical user interface, which may be supplemented with other audiovisual effects. The output of the controller portion can include electrical signals, which are transmitted from the controller housing to electrodes placed on target muscles. In an example embodiment of this system, these electrodes are placed on the gluteal muscles so as to induce- or supplement muscle contraction of these muscles.

An example embodiment of a typical usage session would proceed as follows. The lower back pain sufferer places the stimulation electrodes on prescribed targets on the gluteal muscles. They then secure the resistance band just above the knees. They then apply a force to stretch the resistance band, which will occur with any combination of hip-extension, -abduction and -external rotation. As the band stretches, a sensor is activated, which sends signals to the controller portion. The processing portion processes this signal and then sends a command to the output portion of the controller portion, which in turn produces a prescribed train of electrical impulses, which arrive at the electrodes on the target muscle (e.g., gluteals) such that the gluteals powerfully contract, in some cases with a perceived electrical signal in this region perceived by the user. The signal can be sustained over a prolonged period, ranging from about 2 seconds to up to about 20 minutes (e.g., so as to maintain a strong isometric contraction) or ceased or diminished at a certain point to allow the user to reset the movement cycle (e.g., to allow for concentric cycles of repetitions).

In another application of the same embodiment, the user may begin with the hips relatively internally rotated, flexed and/or adducted (so as to elongate or lengthen the gluteal muscles) as a starting point of the movement cycle. At this starting point, the electrical signal is initiated to produce a contraction of the muscle in the elongated state. The user is then encouraged to shorten the muscle by moving the hip through the opposing movement to the opposite side of the joint. The electrical signal may diminish through the arc of the joint so as to diminish as the user arrives at the opposite point of the joint. This relationship may be inverted or electrical signals may be increased or decreased during the joint range at various points to elicit certain compensatory responses on the part of the user. Tis action is particularly therapeutic for the basal ganglia in the brain, as the user has to control tension against an external source (e.g., which is not a stimulus normally encountered, and which is highly therapeutic in terms of promoting plasticity as well as exposing deconditioned or injured muscle to tension). This is particularly valuable as a way to rehabilitate brain- or spinal cord-injured patients as well as those having been very deconditioned or bed-ridden.

The device as described to this point can be highly effective with lower back pain patients as it simultaneously stimulates motor- and analgesic regions of the central nervous system (CNS). In some embodiments, the application dynamically strengthens pelvic muscles. When these muscle function in normal gait, the overall effect is a marked decompressive effect on the spinal joints. The system can be further supplemented to support the application of lower back pain mitigation (as well as expedited recovery of normal gait, which can have applications in a wide variety of lower limb conditions including post-surgical rehabilitation of hip, knee and ankle as well as pain and symptoms mitigation of a variety of injuries and painful conditions of the lower limbs.)

For the application of lower back pain rehabilitation, a second output system can be coupled to the system controller portion. This system interfaces with the body at the paraspinals via elongated (e.g., approximately rectangular or ovoid) transcutaneous electrodes (e.g., intentionally designed to cover the paraspinal muscles adjacent to the thoracolumbar muscles) This system is activated when the processor directs a therapeutic electrical signal or pattern to the paraspinals, once the gluteals are activated. This is a useful approach wherein higher activity (e.g., exertion on the part of the user) of the gluteals is “rewarded” with increased stimulus directed to the paraspinals. This output to the paraspinal area can be of a level and nature so as to resemble transcutaneous electrical nerve stimulation (TENS) (e.g., activating sensory threshold only) or of a level so as to similarly recruit the paraspinals (e.g., muscle stimulation resulting in contraction). In other embodiments, the output to the paraspinals may be thermal, vibrotactile, light-generating (e.g., including laser light), infrared, or dispense a form of therapeutic agent, which may be chemical, gas, fluid, or any other therapeutic agent. In an example embodiment, the output to the paraspinals is electrical in nature resulting in an oscillating, fluctuating output pattern that alternately contracts and relaxes the paraspinals (which can be relaxing and soothing in many instances of lower back pain, providing a combination of symptomatic relief with apparent strengthening of the paraspinal muscles). While this is the an example pattern, the output to the paraspinals might include any pattern of recruitment including but not limited to sinusoidal, square wave, triangle wave, irregular pattern and sustained contraction over numerous seconds or minutes. The rate of oscillation, in this embodiment ranges from about 0.1 Hz to about 1 Hz in a primary square wave pattern but the rate could be at any range deemed therapeutic or relieving by the user or the therapist directing use of the system.

In yet another embodiment, the second output system can be directed to a particular limb or region in the body. This output energy may take the form of an oscillating-, cyclical- or continuous stimulus. For example, in a treatment method for symptoms or pain in one limb, the output system would be directed to the contralateral limb in order to activate a conditioned pain modulation response. This can be of particular therapeutic value in nerve-based conditions such as complex regional pain syndrome and peripheral neuropathies. In other embodiments, a second or third output can be directed into a specific painful region of the body to exert a form of stimulatory or modulatory energy to the culprit body region.

One of the notable benefits of this embodiment is found in the overall relationship between the user's activity and the resulting response delivered to them by the system. In this system, the user is required to “earn their analgesia” rather than passively receiving it, allowing for a more constructive use of therapeutic electrical stimulation more in line with modern understanding of factors promoting pain and disability. Another surprising effect is a highly positive alteration of bipedal biomechanics (e.g., objectively making changes to the gait cycle and regulation of posture) so as to significantly offload the structures of the spine, thereby providing profound relief both in the short- and long-term. This is a significant benefit over other spine pain therapies, which are directed at the spine itself, rather than the pelvis, the spine's functional foundation One disadvantage of the former approach is while these approaches might temporarily mitigate symptoms from an irritated or damaged spine, they do not prevent recurrence of pain and damage that accumulates as a result of faulty biomechanics, especially of the pelvis and hip over the lower limb during gait.

The topical therapeutic compositions can be used with the stimulation techniques and devices disclosed herein, in some cases. The stretch band embodiments can serve to reduce the biomechanical and central-nervous system components of a pain- or injury syndrome, while the topical compositions can be primarily aimed at local nerves and symptom-producing tissue. When people suffer from lower back pain and other injury syndromes, there are central and peripheral sequelae. This two-pronged approach has been tremendously effective for lower back pain, for example.

The method of treating the human nervous system can be aimed at stimulating the human vestibular system, cerebellum and associated systems associated with postural and movement control and stability. In terms of background to the underlying mechanism of action, the human vestibular system includes semicircular canals in the inner ear, which are aligned to transduce movement around opposing axes, approximately corresponding to conventional x, y, z axes. Also, the otolothic organs (e.g., utricle and saccule) can be activated by sustained postures. There are certain conditions, such as benign paroxysmal positional vertigo, which manifest due to dysfunction in the semicircular canals. Maneuvers called the Epley maneuver, Semont and the barbeque roll have been developed to dislodge or remove particulate that might abnormally be found in these canals through a series of movements In some embodiments, there can be benefit in stimulating (e.g., maximally stimulating) the pelvic stabilizers, as described above, while positioning the head in a series of movements corresponding to each canal of the vestibular system in some cases even in the absence of foreign bodies (e.g., canaliths) within the canals.

The Epley maneuver can be performed as the patient is placed in the seated position In this position, the band would be placed around the lower limbs, just above or just below the knees In one embodiment, the user is instructed to maintain firm tension on the band by abducting and externally rotating the hips (tension is maintained throughout the following body head movements alternatively the user might execute a series of movements against resistance, for example setting and resetting the starting position described). The patient will then turn the head back and to the side, while rapidly lying backward into the supine position with the head fixed in that position. They will then rest in this position for a certain amount of time, dictated by symptoms or prescribed direction. They will then rotate the head into the mirror position on the opposite side and rest there again. They will then roll onto their side with the head remaining fixed. Upon rolling to the side, the user will then slowing rotate their head about 90 degrees toward the floor and then rest there for a short period of time (e.g., typically around 30-60 seconds). The patient will then sit up. At each station the patient will concentration of strongly abducting and externally rotating the hips, thereby activating the gluteal muscles.

The Barbeque roll maneuver can be performed in a similar manner. The patient is supine. They turn their head approximately 45 degrees to the side (e.g., typically the side afflicted by horizontal canalithiasis). The patient will rest here for about 30-60 seconds. They will then turn the head into the mirror image position on the opposite side, again resting at this point for about 30-60 seconds. They will then roll (e.g., in the direction that the head was last turned) to their stomach and rest the head in the midline, again for about 30-60 seconds. They will then turn their head back to the starting position by rolling onto the side of the body (e.g., the side that the head was first turned) then onto their back, after which they will sit up. In each position, the hip extensor and external rotators are strongly activated.

The application of this method has been demonstrated to improve human sensory perception; mitigate bodily sensations (including pain, disequilibrium, visceral symptoms, nausea, anxiety, sleep disturbance, among others); improve motor function (including balance, muscle recruitment, coordination, among others); and improve sense of wellbeing. Without being bound by theory, this effect is believed to be the result of stimulation of the Cerebellum, which has a wide range of functions in the human brain and nervous system. This method has particular applications in fall prevention and brain rehabilitation.

In an example embodiment of this therapy method, as a first (optional) step the user (or supervising therapist) would identify a culprit plane of movement. This is a plane of movement of the body or head that results in production of symptoms or a physiologic response from the body (e.g. elevation in heart rate, change in pupil diameter, onset of dizziness, aggravation of pain, onset of familiar symptoms, movement response in muscle or limb). The user would then associate this plane with a culprit canal and subsequently aim to target that canal with a prescribed maneuver (e.g, resembling Epley maneuver or barbeque roll) but rather than simply passively moving the head and body in sequence in a passive supine, side-lying and prone positions, the user can aimengage (e.g., maximally engage) the pelvic or postural muscles throughout the sequence of maneuvers, using the devices and techniques described herein, and in some cases also by positioning of the user. One particular useful position within this sequence is the prone extension position wherein the user is propped on their elbows, thereby extending their spine in a relaxed manner (e.g., similar in manner to the MacKenzie extension position, wherein the patient is prone, resting on their forearms are extended arms, thereby creating extension of the spine), whilst activating (e.g., maximally activating) the gluteal muscles. This particular position is not only useful as part of the therapeutic sequence for addressing the CNS but also profoundly relieving as a method for the treatment of lower back pain.

Now to a further description of the processor portion of the device system, in some embodiments the processor can include any system or component capable of processing inputs and creating some form of responsive output. Examples include a mobile phone, mobile tablet, laptop- or desktop computer. The processor component may hold an application that serves as the controller for the device system, allowing the user to adjust various functional parameters and functions of the device system. This application may demonstrate the total tension or extent of displacement or deformation in the sensor component of the system. This information can be displayed as part of some sort of game or display the moment-to-moment status of the sensor portion in a symbolic manner that simplifies this information or presents the information in a more engaging manner for the client. One embodiment of the app can show a moment-to-moment comparison between target effort and actual effort. Yet another embodiment may display imagery or deliver audio signals that are specifically intended to promote analgesia, including mindfulness- or hypnosis-based activities: therapeutic sounds or audio frequencies; or brain-stimulating imagery of video material. A particular embodiment of the application can include oculomotor or vestibular exercises, including optokinetic, pursuit, saccade, convergence, vestibular eye movements or other forms of visual stimulation.

In one embodiment, the continuous band loop form can be replaced by a linear portion of the same or similar material (e.g., with no closed loop) such that a wider range of resisted physical activities might be undertaken. This form allows for the user to anchor one end of the band to a fixed or moving point, which may be environmental or on the users body, while the other end is secured or anchored on a part of the users body, such that with a particular movement or series of movements, the band will be deformed in particular directions to provide resistance and to transduce the movement into signals delivered to the system.

In one embodiment of the above method and system, the system can involve an additional component, which is used to assess, improve or guide optimal breathing patterns. One embodiment includes a component that is attached to—or encircles the abdomen so as to be deformed as the user engages in good belly or diaphragmatic breathing or when they engage a good abdominal muscular brace. In this manner as the abdomen expands, the sensor portion is stimulated, giving rise a response into the processor portion. In this application this positive user response is acknowledged or encouraged by a particular response, which may include a audio-, visual- or tactile confirmatory signal; an output from the controller portion such as an electrical signal that results in a change in muscle tone or activity in one or more bodily targets; or the generation of some form of other response that affects physiology or perception. The benefit of this application is that it coaches the user to relax the abdomen, allowing for improved diaphragmatic breathing whilst the gluteals are being stimulated (e.g., maximally stimulated). This application is particularly useful in cases of chronic lower back pain as the user is retrained to engage muscles that offload the spine, whilst relaxing muscles that promote longitudinal compression of the spinal joints.

A System and Treatment Method for Symptom Disorders of the Lower Limbs & Central Nervous System Conditions Affecting Gait

Utilizing a certain embodiment of the abovementioned system, a treatment method for symptom disorders of the lower limbs is described. Such disorders can include one or more of cruciate ligament injury or rehabilitation: ligament sprain or injury; chondromalacia patella; Osgood schlatters disease, disorders of gait: post-fracture rehabilitation, plantar fasciitis; Achilles tendonitis; peripheral neuropathy, or pain in the lower limb. It should be noted that this application and associated system can look and operate in the same manner described herein for the application of lower back pain because improved function of the pelvis can have a positive effect on structures above the pelvis (spine) and below the pelvis (lower limbs). However, a therapeutic application for the lower limbs is further described. This application is characterized with application of transcutaneous electrodes to the gluteal musculature, whilst a form of band, loop or attachment is applied to the lower limbs. This member houses the sensor component of the system. The user is then directed to exert a strenuous and/or consistent effort (e.g. isometric contraction) against the resistance material. This effort can be delivered in the side-lying position in a “clamshell” position but can also be administered in a quadruped, prone, supine, standing, squatting, walking, jumping positions, as a limited list of examples. An example embodiment of this method can use around 5-20 minutes of effort. The as the user engages the system, due to its nature, a responsive signal will be returned to a certain target muscle or group of muscles. This can include at least one gluteal muscle. A surprising finding with the application of this method is that target muscles adapted faster and there was a profound reduction in pain in most cases, even in remote areas of the body but especially in the lower limbs. This method can also be applied to improve physical performance, especially of those activities that involve the lower limbs like cycling, running or jumping. The technique is similar to that described above.

In a certain applications, the procedure described above can be modified so that the stimulating electrodes can be places on the one or more of the quadriceps muscles and the user can be directed to go through normal gait cycle during various patterns and intensities of electrically-induced muscle tension. This can be prescribed when a therapist notices dysfunctional knee flexion during load acceptance and loading phases of the normal gait cycle, which can suggest that knee extensor function may need to be improved.

Therapeutic Method of Alleviating Pain and Body Symptoms Via Manual Therapeutic Technique Utilizing Therapeutic Chemical, Agent

Inflammation and sensitization of superficial nerves of the body is a largely unappreciated and undertreated cause of physical pain and disability. Superficial nerves of the body are particularly prone to irritation and sensitization and can produce extreme levels of pain and disability. When nerves are symptomatic, they are typically edematous, meaning that they are swollen with fluid, which practically increases their size, reduces movement against surrounding tissues, and increases inflammation- and symptom-causing friction against adjacent tissue. Friction or contact with an affected nerve in this state is likely to worsen the inflammation, edema, movement restriction and associated pain and symptoms Current manual, physical therapeutic techniques are limited in a number of ways when it comes to potentially, therapeutically addressing symptomatic inflammation of superficial nerves. Firstly, most manual “soft tissue” techniques (for example massage, tissue release, stretching or tissue mobilization) are not focused enough on the specific pathway of the nerve to liberate the nerve. Secondly, when a nerve is symptomatic or inflamed, any contact with the nerve is likely to provoke pain and inflammation, which can outlast this period of provocation by hours, days or even longer is extreme cases. Thus, there is a need for manual techniques to mechanically liberate target nerves from surrounding tissue in the shortest timeframe possible so as to reduce aggravation of symptoms whilst liberating the nerve from ongoing irritation by surrounding tissue, as is the case when nerves are focally entrapped.

One method of controlling pain is the use of topical analgesics but in the context of neurogenic inflammation, there are a number of important limitations with conventional applications of this type of therapy. Firstly, available therapeutic agents of current drug classes are almost as a rule ineffective for the treatment of pain and symptoms associated with neurogenic inflammation, especially of superficial nerves and many are too risky to be considered for long-term use for this application. Second, with general application of these agents, much of the topical agent is dispersed into areas where it is not needed, which means that much is wasted and less than an optimal amount is directed over the pathway of the superficial nerve. Thirdly, because superficial nerves are somewhat variable in form and may be affected in only some of the potential nerve branches in any single nerve, it is necessary to identify affected areas to better direct subsequent therapeutic interventions. What follows is a therapeutic method for treating symptoms and conditions stemming from nerve irritation.

Therapy Step 1) Target nerves and tissues are identified: The target portions or branches of nerve are identified by integration of knowledge of local neural anatomy, palpation, and/or movement provocation. It is possible to limit this step to palpation alone. A system of diagnostic palpation is described. The symptomatic region of the body is identified (primarily by correlation with the patient's report of pain and other symptoms.) Within the painful area lightly press (e.g., palpate) and lightly move superficial tissue specifically to identify focal “sore spots”; small palpable nodules (that feel like pellets, which may not be painful); multiple “corrugated” strips (that feel like strings of pearls); focal areas where superficial tissue is resistant to movement. The examiner will then typically recognize the pattern of these points (which may be likened to a dot-to-dot activity wherein the cumulative spots imply a particular nerve) and associate them with a known nerve or nerves.

As an alternative to this step, a particular “montage” of nerve sites are identified based on the practitioner's experience and expectation that they would produce a positive effect in ameliorating the particular symptoms of a particular condition when addressed with the therapeutic method described herein.

1b) Montage of target nerve sites can be identified based on patient's particular condition or symptomatic presentation: In certain instances, there may not be an obvious painful or symptomatic configuration of one or more nerves to guide next steps. One example of such an instance would be in cases where an unskilled person is required to apply a therapeutic topical or therapeutic sequence. Another example might be in cases where the patient's symptoms have temporarily abated (e.g. interictal stage) where symptoms do not sufficiently guide next steps. In such cases, a prescribed montage or pattern of application sites, based on target nerves is called for. A more detailed description of condition-driven montages is described in the next section.

The next step in this process could be either 2a) or 2b). Whichever of steps 2a) or 2b) is performed first, the other of steps 2b) or 2a) can be performed next. That said the steps could be performed in any combination or sequence.

Therapy Step 2) Therapeutic Compound is Applied to Target Regions of Nerve: The topical therapeutic utilized for this application may be any topical therapeutic with analgesic, anesthetic or healing properties, including those described herein, optionally but not necessarily including at least in part of 5-40% of total volume dextrose or mannitol, optionally but not necessarily buffered by an alkalizing agent (potentially including one or more of sodium bicarbonate, sodium citrate, L-Arginine, sodium hydroxide), is applied to portions of a superficial nerve distribution (e.g., using one or more of the following manual techniques, specifically intended to mechanically liberate superficial nerves and enhance the synergistic effect between the applied manual technique and topical therapeutic agent). It has been found that the benefits of this integrative approach include less soreness after manual techniques (a previous problem with applying manual techniques to inflamed nerves and injured tissue); more efficient delivery of therapeutic topical agents (applications are faster and require less volume of topical therapeutic); vastly more effective response to topical applications (by combining movement and better targeting the agents, the patient benefits from the activation of endogenous analgesic systems in the CNS; nerves are mechanically liberated thereby breaking the cycle of relief and aggravation, pain relief is more consistent and durable); and patients are empowered to move more, thereby mitigating disability and promoting activity and self-efficacy.

Any of the following steps and techniques can be (optionally) markedly enhanced by simultaneously or sequentially, grossly mobilizing target nerves and adjacent tissues by one or more of the following: intentionally moving the limb and nearby joints that the target area is situated in by cyclically opening and closing a nearby joint, by moving the target limb to end-range to create a stretch, or by performing a (previously) provocative physical maneuver. The following steps (2a-e) represent a method for applying topical therapeutic agents, which markedly improves efficacy. It has been found that these actions markedly improve efficiency and effectiveness of pain mitigation and liberation of symptomatic superficial nerves. As previously mentioned, inflamed nerves are edematous (swollen) and therefore restricted. With the delivery of an appropriate topical therapeutic, this swelling can diminish rapidly, which assists in mechanical liberation of the target nerves and tissues. The following therapeutic steps can be applied (e.g., in the sequence they are listed in, in any other order, or any subset or combination thereof):

2a) In an effort to better direct topical therapeutic application (rather than applying like a typical lotion or topical), if “small sore points” or small palpable spots are identified, the topical agent can be focused into these “hot spots” (without being bound by theory, it is believed that these likely represent terminal branches of nerves where the nerve is interfacing with other tissues).

2b) In an effort to better direct topical therapeutic application (rather than applying like a typical lotion or topical), the topical agent can be delivered in a longitudinal pattern (typically superimposing the skin above superficial nerves) along a series of palpated nodules (“string of pearls”), focusing on the “corrugated feeling” areas (without being bound by theory, it is believed that these are caused by constriction points along pathway of superficial nerves).

2c) In an effort to better direct topical therapeutic application (rather than applying like a typical lotion or topical), topical agent can be applied in a “pinch, pull and roll” maneuver wherein the skin overlying a superficial nerve pathway (pre-treated with topical agent) is secured between the fingers and lifted upward (pulled away from deeper layers) in a maneuver that approximately resembles a ‘cigar-rolling’ or dough-rolling maneuver between the fingers. Optionally, this maneuver can also be used to simultaneously and efficiently deliver more topical agent into the area of the target nerve. This particular movement technique has the intentional, dual-purpose of mechanically liberating the nerve whilst delivering the compound to the target nerve. It has been demonstrated via ultrasound that this is particularly effective in targeting the nerve.

2d) In an effort to better direct topical therapeutic application (rather than applying like a typical lotion or topical), topical agent can be applied first to the target nerves and/or tissues. A downward pressure is applied over this site with a soft manual surface (e.g. thenar eminence, palm, or hypothenar area). Whilst a downward (e.g., superficial to deep) pressure is maintained over the target area, the underlying tissues are mobilized by opening and/or closing one or more joints of the target limb, in a cyclical or repetitive manner. The manual contact surface may optionally be maneuvered in small cyclical movements that are circular (twisting) or linear (either parallel or transverse to the plane of the limb). In a certain application, one hand may be substituted for two, in which case, it can be useful to place the hands on either side of the target nerve pathway. The objective of this maneuver is to relatively arrest certain tissues, whilst allowing the target nerve or tissues to break free whilst efficiently delivering the topical therapeutic agent in an effective manner (vastly superior than just “rubbing it into the skin”).

2e) In an effort to better direct topical therapeutic application (rather than applying like a typical lotion or topical), the agent can be applied to one or more sites or the entire accessible length of one or more superficial nerves.

The abovementioned topical application steps (2a-2e) can be (optionally) delivered by a mechanical device or other device, intended to replace the human hand, such as those described herein or any device capable of delivering comparable physical actions to the target tissue.

The abovementioned topical application steps (2a-e) can be (optionally) replaced by the application of an intentionally formed interface with the skin, which contains and/or delivers a therapeutic compound to the skin (e.g, replacing the need to deliver this compound by hand or device.) Examples include adhesive- or non-adhesive: bandages, medicated plasters, patches or similar structure capable of interfacing with the skin surface with the intention of delivering, protecting or enhancing delivery- or therapeutic effect of topical therapeutic agents.

It should be noted that for most patients, performing or self-performing steps (2a-e) is typically sufficient to obtain sufficient or complete relief from pain- and symptoms. The topical application steps can be enhanced through the use of non-absorptive gloves (e.g. vinyl, latex, non-latex) such that no topical is absorbed into the hand and grip with the skin surface is enhanced. Thus, it may not be necessary to perform any of the next steps in the therapeutic process. Furthermore, in certain instances, it may not be advisable, efficient or comfortable to perform steps 3-5. For example, a particularly patient may be in pain that is too severe or the body tissues may not be accessible or the patient may be self-administering a topical therapeutic over a single or multiple sessions in order to prepare for movement.

Therapy Step 3) A Tissue-liberating maneuver is performed: Once the topical therapeutic has been delivered to the target nerves, the therapist may decide that further tissue liberation is justified. The therapist may also optionally decide to perform this step (3) ahead of step (2) or in an isolated manner without (2), although in some instances this is not preferred as movement and direct manual techniques applied to sensitized nerves is markedly more uncomfortable if symptom-producing nerves have not been pre-treated. The following description assumes that step (2) has been performed and the presented sequence of steps is being administered. Once the target nerve region is identified one or more of the following maneuvers are delivered to the nerve distribution.

Technique 3i) Parallel or perpendicular traction: This technique is similar to (2c), the primary difference being that in this technique there is no application of topical therapeutic. Using one or more hand (or suitable device or tool), the therapist secures the tissue overlaying and/or adjacent to the target nerve region in a pincer-type grip (e.g., the tissue grip can secure a tissue fold either parallel to—or perpendicular to the path of the nerve). An example of a potential tool to replace the hand for this application includes a suction cup (of a nature similar to traditional suction cups associated with traditional Chinese medicine). With this tissue secured, the therapist may optionally perform repetitive, cyclical, or oscillatory maneuvers of short- or long-range, or maximally stretching (e.g., elongating) of the target nerve(s) and surrounding tissue by inducing a stretch on the limb hosting the target tissue. This is intended to mechanically liberate the target nerves. These movements can be created by opening and closing one or more nearby joints; taking the limb into a stretch by opening a nearby joint to end-range (this stretch can be static and sustained or cyclical and repetitive).

Technique 3ii) Nerve tracing maneuver: using one or more digits (or some form of manual tool or device), an initial downward (e.g., superficial-to-deep) pressure is applied to a certain point along the pathway of a target nerve, then, as the downward pressure is maintained, the contacting digit (or tool or device) surface is dragged along the pathway of the nerve so as to trace it's path. This technique may be optionally enhanced with the body movement as described in Technique 3i).

Technique 3iii) Blunt friction, using a blunt surface, such as the length of one of the bones of the fingers (or a blunt tool or device), an initial downward (e.g., superficial-to-deep) pressure is applied to tissue over a portion of the nerve. This region can be selected by its symptom- or pain producing characteristics and/or its location, for example a distal portion of the nerve or a region where no painful nodules can be identified but rather a region producing an ill-defined symptoms- or pain, which may or may not be identified by dense or edematous tissue. Maintaining this downward pressure, small, cyclical, repetitive movements are performed in the transverse plane (e.g., parallel to the skin surface), which can be linear or circular in nature. This can be applied for around 20-80 seconds—a period of time, which may be continuous or interspersed with periods of no-movement at all or just downward pressure (without friction on the target tissue). The frequency of this repetitive movement can be of any suitable frequency, however, an example frequency for a manually-delivered technique is around 1 Hz. Device-delivered frequency, optionally ranges between around 1-53 Hz but may reach frequencies of about 150 Hz in some cases. This technique may be optionally enhanced with the body movement as described in Technique 3i). This technique can also be thought of as the opposite of “Negative pressure hold and oscillate” (described herein) in the sense that the forces applied are similar, except that in this technique a downward pressure is applied to the skin in a compressive motion whereas in “Negative pressure hold and oscillate” there is a traction force applied in the opposite direction to decompression tissue.

Technique 3iv) Negative pressure trace. Once a portion of tissue is secured over the nerve with the hand (as in Technique i) or with some form of device (potentially including a form of suction cup), the point of body contact (e.g., the hand or device interface with the skin) is moved or “traced” along the nerve pathway as if the nerve represents a path to be followed by the skin contact point. As this contact point advances along its path, it should be noted that the actual tissue arrested by the hand (e.g., fingers) or device will change but the general form and pressure of the contact point can be maintained. This maneuver can be supplemented with limb movement (as in 3i). Specifically, some embodiments of this technique include: a) a negative pressure contact point is developed by hand or device while the patient stretches the target limb and tissues by opening a joint to tolerable end-range and either holding it there for a sustained period of time, or cyclically moving into- and out of end range so as to create an oscillating stretch; b) a negative pressure contact point is developed by hand or device after which the patient cyclically moves the target limb (or another limb) through a prescribed range so as to amplify mobilizing forces on the target nerve(s) and surrounding tissues; c) a negative pressure contact point is developed by hand or device after which the patient is instructed to perform a particular physical maneuver, which might include a natural movement like walking or running; a prescribed movement like a squat or lunge; or a portion of a movement or joint range. In a typical application, the movement selected in c) would be a movement that was (prior to application of therapy) known to be provocative with respect to pain- or symptoms.

Technique 3v) Negative pressure hold and oscillate: a portion of tissue is secured over the target nerve with the hand (as in Technique i) or with some form of device (e.g., some form of suction cup). This point of body contact is either a) left alone for a period of time with no movement; b) left alone while the patient moves the actual limb and/or body in a natural or prescribed movement (as described herein), c) the portion of the hand or device that is contacting the secured skin site is cyclically moved in one or more plane of movement (e.g. transverse linear, transverse circular, perpendicular (e.g., deep and/or superficial) (for example, light percussion) This movement can be quite small in amplitude and developed in order to intentionally mobilize the underlying nerve and mobilize the tissue overlaying the nerve.

At times, it may be beneficial to create multiple contact sites in a single limb to address multiple nerves or multiple sites along a nerve pathway. At other times, it may be beneficial to treat one- or more limbs simultaneously with one- or more-contact sites. These therapeutic contact sites can include any combination of manual or device-assisted applications.

Therapy Step 4) Re-assessment: Having delivered one or more of the abovementioned techniques, the patient is re-assessed for response. In some instances, it is not necessary to repeat any steps of the treatment, but in some cases, it may be beneficial to repeat one- or more (or any combination) of the abovementioned steps, such as when the patient has failed to respond as expected, responded incompletely; or the primary symptoms have resolved, thereby making other (previously unnoticed) symptoms noticeable. This step is optional.

Therapy Step 5) Continued Topical Therapeutic Application: Having determined a favorable response from one or more of the preceding steps, the patient is instructed to continue to apply the particular topical therapeutic agent to target nerves and tissues with or without applying the abovementioned therapeutic techniques as “home care”. This step is optional.

Methods for Prophylactically and Acutely Treating Symptom Disorders

The abovementioned therapeutic method of intentional administration of topical therapeutics to (and associated with mechanical liberation of) target nerves can be applied in a variety of conditions, many of which have not typically or conventionally treated with topical therapeutic administration (or at least not in a manner that prescribes the application of topical therapeutics directly to superficial nerve sites). The inventor has noted significant success in a therapeutic approach that emphasizes administration of topical therapeutic, in the manner described above, in each of the following specific conditions. In each instance, this application represents a beneficial treatment method.

The therapeutic method might also include a series of steps prior to exercise or physical exertion wherein a therapeutic compound is applied along the pathways of superficial nerves in order to counteract the effects and perception of fatigue, in order to improve physical performance. In one embodiment, the therapeutic compound would consistent of an amount of sugar (or sugar alcohol). In another embodiment, the compound would consistent of an amount of sugar (or sugar alcohol) in conjunction with one or more alkalizing agents (e.g. sodium bicarbonate and sodium hydroxide).

Therapy Method for Treatment of Headache and Craniofacial Pain Conditions and Symptoms

The following therapy method is developed for headache and craniofacial pain conditions (incl. cluster headache, sinus headache, migraine, exertional headache, hormone headache, tension headache, facial neuralgias). Extracranial allodynia is a disabling condition experienced by many migraine sufferers. Common migraine therapies like the Triptan class of drugs are ineffective in the patient with allodynia. During development of this therapeutic method, the approach taken was of resolving extracranial allodynia in an attempt to discover whether this would modify the primary condition (migraine headache) itself. The response was highly favorable and confirmatory.

In consideration of the technical method described above, a method for treating headache- and cranial disorders and symptoms is hereby described. Current and conventional therapies for treating headache disorders, such as tension headache, cluster headache, and migraine, are typically directed at the central nervous system (CNS), or, in the case of some “alternative” or physical therapies, directed at structures of the musculoskeletal system (e.g. muscle, fascia, tendons, or joints of the spine). Some approaches stimulate peripheral nerves of the face and head in order to create afferent nerve signals that create CNS effects that attempt to mitigate headache symptoms. The following therapeutic method simultaneously a) mechanically liberates superficial nerves, b) mitigates neurogenic inflammation, c) stimulates target centers in the CNS, d) therapeutically stimulates peripheral nerves, in a single application What is reported next is a treatment approach that is directed at peripheral nerves of the head and cervical region in order to elicit a local effect and positive state change on the peripheral nerves themselves (rather than simply creating afferent impulses in peripheral nerves, in order to affect the CNS).

Consistent with the therapy method, the first step of this therapeutic method involves either the selection of a particular montage of superficial nerves (described herein) or the identification of tender or painful points in or in the region of one or more of the following nerves, supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erbs point, supraclavicular, lateral supraclavicular and intermediate supraclavicular. In an example application, each of these nerves is addressed bilaterally. In another application, any one or combination of these nerves, unilaterally or bilaterally might be selected. In one embodiment of this application, the treatment method can be applied as an acute therapy when the patient is manifesting an actual headache disorder or symptoms- or associated-symptoms of a headache disorder. In another embodiment, the treatment method can be applied interictally (between headache attacks) as a prophylactic therapy.

In a certain embodiment of this therapeutic method, the user can be directed to apply this method in a prescribed program, which can be once or twice per day (or any other frequency deemed appropriate), in order to reduce symptoms of any nervous system condition, especially those associated with headache or head symptoms. An embodiment of this method will direct the user to first apply the method to the nerves within the location of the head that the user typically experiences their headache. For example, if a user where to frequently experience migraines on the left side of the head, over the left eye, then the user would be directed to treat those nerves around the orbit of the eye (potentially including supraorbital, supratrochlear, or infratrochlear), such that a dose of physical energy and therapeutic topical compound is directed to these nerves.

The benefit of this method is that the nerves are simultaneously mobilized (reducing the ability of surrounding tissue to provoke the nerves); rendered non-painful by administration of a therapeutic compound (thereby reducing their capacity to prime the CNS systems associated with head pain and symptoms):and through administration of energy to the peripheral nerve, the CNS is simultaneously activated, building plasticity in endogenous analgesic centers. This method is thus addressing the peripheral nervous system (PNS) and the central nervous system (CNS) with the overall benefit of reducing headache intensity and frequency. An example utilization of this technique is in a prophylactic paradigm (that is, the user utilizes the method between symptomatic events in order to positively affect their condition), however, this method can also be utilized within an acute paradigm, that is to mean utilized during symptomatic events (e.g. headache) in an effort to abort a particular headache or head symptom attack.

In another application embodiment, once tenderness, restriction of tissue movement or mild edema (or any of the symptoms) are noted in the region of any of these nerves, priority is assigned to these nerves. Priority is intended to mean that the therapist or individual utilizing the method will treat these areas first, potentially spending relatively more time of the treatment session delivering therapeutic technique application to these points.

This therapy method dictates that these points can be therapeutically addressed with any one or more of the therapeutic technique methods, described in this document, in any combination. 1) a manual technique; 2) a technique using a tool or device (e.g., the devices described in this document); 3) a technique utilizing a therapeutic interface with the skin; 4) a method combining excitatory and inhibitory topical compounds, 5) a technique utilizing a therapeutic interface with the skin (e.g., an interface adhered to the skin that delivers or enhances delivery of a topical compound and/or therapeutic energy).

In one embodiment of this method, a manual technique or sequence of manual techniques (e.g., as described in this document) is directed at a portion of one or more of the supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erb's point, supraclavicular, lateral supraclavicular and intermediate supraclavicular nerves. In an embodiment of this technique each nerve is addressed bilaterally but in certain instances, it may be appropriate to only address a single, unilateral branch of the nerve (these nerves are all paired, bilateral nerves). In an embodiment of this method, emphasis will be placed on the most tender of these sites and each site will be addressed so as to reduce the noxious neural traffic to the CNS. This method can be utilized as an acute or prophylactic therapy.

In another embodiment of this method, a device-administered technique (as described in this document) is directed at a portion of one or more of the supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erb's point, supraclavicular, lateral supraclavicular and intermediate supraclavicular nerves. In an embodiment of thus technique each nerve is addressed bilaterally but in certain instances, it may be appropriate to only address a single, unilateral branch of the nerve (these nerves are all paired, bilateral nerves) In an embodiment of this method, emphasis will be placed on the most tender of these sites, and each site (or subset of sites) will be addressed so as to reduce the noxious neural traffic to the CNS. This method can be utilized as an acute or prophylactic therapy.

The general, device-administered technique dictates the following sequence: 1) target nerves are identified; 2) an amount of topical therapeutic agent is dispensed by the device system; 3) an amount of tissue overlaying the target nerve is arrested, depressed or tractioned by the device (e.g., as part of the device actuated version of the manual techniques described herein); 4) a mechanical, nerve-liberating technique is delivered via the device; 5) and/or a form of therapeutic energy is dispensed via the device to the target nerve as—or after—a prescribed therapeutic mechanical action is delivered to the target tissue, which in some cases also aids in topical therapeutic delivery. The therapeutic mechanical action can take the form of vibration, oscillation, transverse or vertical friction, or any form of kinetic energy that causes superficial- or deeper-tissues to move in a therapeutic manner. In certain instances, the topical therapeutic can contain elements to facilitate electrical transmission, such as silver-containing elements, which has the benefit of making this application much more comfortable, requiring lower intensity of electricity and enhancing the transmission of therapeutic energy. An example form of therapeutic energy used for this application is electrical (possibly but not necessarily of a direct, continuous, monophasic, and/or negative form). Another form of therapeutic energy may take the form of light energy (e.g. cold laser or infrared light). As described herein, the body-interfacing surface of the device is intended to make firm contact with the overlaying skin, which can take the form of a flat interfacing surface (e.g., resembling an angled spatula) but may also be of any other form intended to contact or arrest the tissue in an intentionally way. These potential forms might include a roller-ball, a multi-roller-ball (more than one interfacing mobile spheres), a suction surface (intended to exert a negative pressure on the skin in order to traction the superficial tissues overlaying the target nerve), or a textured surface (suited to increase friction and grip with the skin).

In one embodiment of this therapeutic method, a therapeutic interface delivering a therapeutic topical compound and/or therapeutic energy is utilized, intentionally formed to cover one more of the following nerves, the supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erb's point, supraclavicular, lateral supraclavicular and intermediate supraclavicular nerves. In an embodiment of this technique each nerve is addressed bilaterally but in certain instances, it may be appropriate to only address a single, unilateral branch of the nerve (these nerves are all paired, bilateral nerves). In an embodiment of this method, emphasis can be placed on the most tender of these sites, and each site (or subset of sites) can be addressed so as to reduce the noxious neural traffic to the CNS. This method can be utilized as an acute or prophylactic therapy.

In another embodiment of this headache-disorder and -associated symptom treatment method, a therapeutic technique utilizing a combination of excitatory and inhibitory (similar to the “excitation-inhibition technique” described herein) therapeutic agents and/or energetic stimuli, as previously described in this document, are utilized. Thus combination of appealing to the autonomic nervous system using an excitation-inhibition approach can be supplemented in certain embodiments with the application of therapeutic agents to other accessible nerve sites. For the purposes of this particular application, directed to affect headache and/or craniofacial conditions and associated symptoms, these additional nerve sites can include one or more of the following: the supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erb's point, supraclavicular, lateral supraclavicular and intermediate supraclavicular nerves.

In the context of this application, a combination of excitatory- and inhibitory-agents are applied in any combination (e.g., utilizing topical-, transcutaneous- or transdermal-delivery), to one or more autonomic-associated nerve sites. With respect to parasympathetic-associated sites, excitatory- or inhibitory-therapeutic agents (potentially replaced by—, supplemented with—or delivered in conjunction with other forms of therapeutic energy), can be applied to parasympathic cranial nerve sites unilaterally or bilaterally at one or more of the following sites the concha, tragus and ear canal of the ear, lateral surface of the neck.

In conjunction with the abovementioned, parasympathetic-associated nerve sites, in a particular embodiment of this therapeutic application, inhibitory chemical agents (e.g., utilizing topical-, transcutaneous- or transdermal-delivery) and/or therapeutic energies (potentially including one or more of electrical, mechanical, light, sound or ultrasound, or thermal energy) might be applied to one or more of the following sites, unilaterally or bilaterally: the sphenopalatine ganglion (SPG), the superior cervical ganglion, the middle cervical ganglion, and/or the inferior cervical ganglion. The inferior ganglion may be fused with the first thoracic ganglion to form a single structure, the stellate ganglion, just adjacent to C6 and C7.

The SPG can be accessed with a simple cotton applicator (e.g., around 10 cm in length) or by a probe extension to the device systems described herein, which would intentionally deliver a topical, transcutaneous or transdermal therapeutic compound as well as a form of therapeutic energy. For example, the applicator can be inserted into one nostril and advanced to the area near the SPG high in the nasal passage. An alternative embodiment to this method would utilize the device platform described previously in this document, which combines the dispensing of therapeutic agent with a second form of energy directed to the tissues of the nasal passages. As previously described the therapeutic medium might contain conductive elements, which would serve to distribute electrical stimulation high in the nasal passages, which would otherwise be difficult and uncomfortable to reach with direct contact.

The therapeutic agent in this case could also take the form of a topical-, transcutaneous- or transdermal therapeutic disclosed herein, such as including one or more of a portion of sugar or sugar alcohol (e.g., dextrose or mannitol) in the volume range of about 3-45% (although other ranges are possible); an alkalizing agent (e.g. sodium bicarbonate; sodium citrate; calcium citrate; sodium hydroxide, TEA:L-Arginine; Triethanolamine); an analgesic agent; an anti-inflammatory; a polyphenol: a cannabinoid, or any combinations thereof.

An inhibitory agent utilized for this application may also take the form of a compound containing ions known to affect neural conduction, such as calcium and magnesium. One example of an electrical energy utilized as inhibitory agent for this application can include, for example, a continuous, direct, monophasic, negative wave; an electrical signal of 0.05 ms duration (square wave) tonic stimulation by a single stimulus or phasic stimulation by phasic stimulation by repetitive stimuli at 10 Hz train (or in a range of 0.25-200 Hz), lasting 1000 ms (or a range of 100-10,000 ms) and/or 40 Hz, lasting 500 ms can be applied as an inhibitory stimulus as target nerves sites.

While this embodiment of this therapeutic approach is intended to address disorders of the head, neck and cranium (e.g. craniofacial pain syndromes), addressing the abovementioned sympathetic and parasympathetic sites, it can provide benefit in other conditions, including but not limited to Chronic regional pain syndrome types I and II to the upper extremities (CRPS I and II); vascular insufficiency/occlusive vascular disorders of the upper extremities (including Raynaud's disease), improvement of the blood flow following surgical graft or other vascular surgery; poor lymphatic drainage and local edema of the upper extremity following mastectomy; postherpetic neuralgia; phantom pain, CRPS of the breast and pain following mastectomy; tinnitus and hearing disorders, hyperhidrosis of the upper extremity; cardiac arrhythmias; ischemic cardiac pain; neuropathic pain syndromes, especially in cancer pain, seizure (epilepsy) and behavioral conditions (incl. anxiety, post-traumatic syndromes, and depression).

Therapy Method for Treatment of Various Pain and Injury Conditions and Symptoms

The therapy methods described herein can be applied as a therapeutic method for a variety of conditions, some of which are described below, along with the particular nerve sites that might be utilized specifically for each condition. As described above, these sites can be therapeutically addressed with any one or more of the therapeutic technique methods, described in this document, in any combination: 1) a manual technique; 2) a technique using a tool or device (e.g., the devices described in this document); 3) a technique utilizing a therapeutic interface with the skin (e.g., an interface adhered to the skin that delivers or enhances delivery of a topical compound and/or therapeutic energy); 4) a method combining excitatory and inhibitory topical compounds.

In technique methods described herein, a variety of methods can be employed as described in this document but these techniques generally involve the following common steps.

1) The identification of target nerves (or portions of nerves), typically symptomatic or painful and typically identified by a form of mechanical provocation (e.g. pressing, rubbing, tugging, pinching, palpating).

Finding symptomatic nerve sites can be achieved in several instructive ways. The most helpful is to ask the user to feel for any tender or sore spots, little granular balls, any hot spots: any little areas of tenderness or pain, any boggy or edematous spots; any fibrous or granular areas/spots/nodes; series of little nodes that feel corrugated or like a string of beads or pearls. They can also be directed to tap around the painful or affected area and feel for any zinging, electric-shock-like, or painful spots or with a little more knowledge of nerve anatomy, they can be directed to tap or palpate along a known nerve pathway to locate symptomatic sites (e.g., these sites are typically sites of neurogenic inflammation and ideal targets for the application of this therapeutic technique.

The patient can also be directed to provoke a particular nerve via fractioning it in a limb position that is known to traction the nerve. For example, wrist flexion/extension would provoke median nerve; elbow flexion the ulnar nerve; pronation/supination—the radial nerve; wrist adduction or abduction—superficial radial; hip or trunk flexion/extension—the cluneal nerves; hip abduction or adduction the iliohypogastric nerve; ankle eversion posterior tibial nerve and branches; plantarflexion of ankle the superficial peroneal; hip extension the femoral. This list is not complete but illustrates the point that limb movement can provoke particular nerves and their branches, which will serve to implicate these nerves as symptomatic and then direct subsequent treatment of these nerves by the techniques described.

Typically in an area of nerve inflammation, there is a general perception of pain or discomfort by exploring within this area, the patient can be taught to locate tiny superficial nerves and better direct the therapeutic method or therapeutic topical agent. This can be a useful tool to better direct the application of a topical therapeutic that is intentionally developed to address nerve symptoms and contrary to the typical user's instinct to simply spread the topical therapeutic over the general area, which will not deliver the optimal dose of the topical therapeutics to the target nerves and tissues.

The target nerves can also not be overtly symptomatic and might be located by prior anatomical knowledge and accessed by specific instruction. The patient or a medical practitioner could perform the method.

2) The application of either a positive- (e.g., pressing, pushing, rubbing, frictioning) or negative-pressure (e.g., pulling, tractioning, tugging, lifting of body tissue) manual or device-delivered technique. The technique can be characterized by repetitive oscillatory forces or movements or long, sustained force or movement delivery. The primary goal is to liberate nerves and their surrounding tissue to diminish the mechanical irritation of these nerves (the irritation that comes of friction by surrounding tissues).

When directed by hand, such application can take around 30 seconds to 5 minutes to complete. When using any of the device embodiments described above, around 15 seconds to 2 minutes, for example.

In some embodiments, there is an optional application of a topical therapeutic as described throughout this document. An example embodiment of such a topical would include some form of sugar or sugar alcohol or combination of both (e.g., the synergistic benefits of combining these is discussed herein). In another example embodiment, the sugar, sugar-alcohol-, (or combination of both)-contemning compound would be delivered in a medium that is relatively more alkaline than the target tissue so as to induce a buffering effect to slightly- or markedly raise local pH.

Another example embodiment of the therapeutic topical compound can be one containing an amount of fruit- or plant extract or combination of various fruit or plant extracts (with or without the aforementioned sugar, sugar-alcohol or buffering agent). Examples include, Adaptogen or adaptogen extract (e.g. Rhodiola rosea Extract (e.g., CAS No: 97404-52-9), Kakadu Plum Extract and/or oil (e.g., CAS No: 56-81-5, 7732-18-5, 1176234-54-0, 122-99-6), Acai Fruit Extract (e.g., CAS No: 879496-95-4), Salvia miltiorrhiza Bunge (danshen) (e.g., CAS No: 90106-50-6), Polygonum cuspidatum (Japanese knotweed extract) (e.g., CAS No: 501-36-0), Filipendula vulgaris (dropwort) (e.g., CAS No: 90028-75-4), Fraxinus rhynchophylla extract (e.g., CAS No: 8001-73-8), Prunus persica extract (e.g., CAS No: 84012-34-0), Rosa rugosa (rose) (e.g., CAS No: 92347-25-6), Meadowsweet Extract (e.g., CAS No: 84775-57-5), Pyrus malus Flower- and bark extracts (e.g., CAS No: 85251-63-4), Triticum vulgare seed extract (e.g., CAS No: 84012-44-2), Hordeum vulgare (barley) seed extract, Panax ginseng root extract (e.g., CAS No: 84650-12-4), Grape seed (Vitis Vinifera) oil (e.g., CAS No: 8024-22-4), Grape Extract (e.g., CAS No: 84929-27-1), Licorice Root Extract (e.g., CAS No: 84775-66-6), Blueberry (Bilberry) Fruit Extract (e.g., CAS No: 84082-34-8).

An example embodiment would include an amount in the range of about 0.25-15% of any combination of one or more the following: Blueberry Fruit Extract (e.g., CAS No: 84082-34-8), Acai Fruit Extract (e.g., CAS No: 879496-95-4), Red Grape Skin Extract (e.g., CAS No: 11029-12-2), Plum Extract (e.g., CAS No: 56-81-5, 7732-18-5, 1176234-54-0, 122-99-6), Bilberry Extract (e.g., CAS No: 879496-95-4), Pterocarpus marsupium (kino bark) extract (e.g., CAS No: 84604-08-0).

A therapeutic method can include applying a plant extract-containing composition along known nerve pathways and/or symptomatic nerves or nerve fibers to mitigate symptoms of neurogenic inflammation or neuropathy, as well as the combination of this application with manual techniques aimed at alleviating nerve symptoms and/or mobilizing neural or perineural tissues.

For Tinnitus and other hearing disorders, one or more of the following sites can be addressed: cervical cutaneous (e.g., transverse cervical), anterior supraclavicular, posterior supraclavicular, middle supraclavicular, auriculotemporal, posterior auricular, branches of the facial nerve, supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erbs point, supraclavicular, lateral supraclavicular and intermediate supraclavicular.

For knee pain (e.g., including disorders such as chrondromalacia patella, Osgood schlatter's syndrome, post-surgical knee pain, post-ligamentous injuries), one or more of the following sites can be addressed: lateral femoral cutaneous, femoral, oburator, saphenous, superficial peroneal, deep peroneal, posterior tibial, femoral, posterior femoral cutaneous, tibial and peroneal.

For peripheral mononeuropathies, a number of specific sites can be addressed, in accordance with the particular nerve involved: median nerve (for carpal tunnel syndrome): ulnar nerve (for ulnar neuropathy at the elbow and/or wrist): radial nerve (for radial tunnel or supinator syndrome); superficial radial nerve (for cheiralgia paresthetica or superficial radial neuropathy); posterior tibial nerve and plantar branches (for posterior tibial neuropathy or tarsal tunnel syndrome): superficial—and deep peroneal—for peroneal neuropathy at ankle or knee.

For Peripheral Polyneuropathy (e.g., including diabetic peripheral neuropathy, chronic inflammatory demyelinating polyneuropathy, small fiber neuropathy, idiopathic peripheral neuropathy), one or more of the following sites can be addressed. (for lower limb symptoms) lumbosacral nerve roots (especially via the posterior ramus), saphenous, sural, superficial peroneal, deep peroneal, posterior tibial, interdigital branches, lateral- and medial plantar, calcaneal, femoral, tibial and peroneal.

For Lower Back Pain Conditions (e.g., including radicular lower back pain, post-surgical pain, idiopathic lower back pain), one or more of the following sites can be addressed, lumbosacral nerve roots (e.g., especially via the posterior ramus), superior-, middle- and inferior cluneal, lateral femoral cutaneous, oburator, saphenous, sural, superficial peroneal, deep peroneal, posterior tibial, interdigital branches, lateral- and medial plantar, calcaneal, femoral, subcostal, ilioinguinal, iliohypogastric, genitofemoral, posterior femoral cutaneous, tibial and peroneal. With respect to radiculopathy symptoms (e.g., symptoms of radiating limb pain conventionally associated with spinal nerve root irritation), a therapeutic method is described wherein the suspected lumbar disc level is decompressed (e.g. by spinal extension, traction or other form of offloading the lumbar disc), the suspected nerve root is decompressed (e.g., by the aforementioned methods) and some or all of the peripheral nerves associated with the suspected nerve root(s) is treated by the abovementioned therapeutic method. For example, if the L4 nerve root distribution is suspected, the femoral, saphenous nerves can be addressed (at multiple points along their path).

For Hip Pain (e.g., including IT band syndrome, bursitis, post-surgical pain) and posterior thigh pain (e.g., including radicular pain and “hamstring” conditions), one or more of the following sites can be addressed: lumbosacral nerve roots (e.g., especially via the posterior ramus), lateral femoral cutaneous, obturator, femoral, superior-, middle- and inferior cluneal, subcostal, ilioinguinal, iliohypogastric, genitofemoral posterior femoral cutaneous.

For Plantar Fasciitis (and other foot and ankle conditions), one or more of the following sites can be addressed: lumbosacral nerve roots (e.g., especially via the posterior ramus), tibial nerve, peroneal nerve (e.g., including deep- and superficial-branches), sural nerve, posterior tibial nerve, lateral- and medial-plantar nerves, saphenous, interdigital branches, and calcaneal branch.

For Neck Pain (e.g, including whiplash syndrome), one or more of the following sites can be addressed: cervical cutaneous (transverse cervical), anterior supraclavicular, posterior supraclavicular, middle supraclavicular, auriculotemporal, posterior auricular, branches of the facial nerve, supraorbital, supratrochlear, infratrochlear, external nasal, lacrimal, zygomatical temporal, zygomaticofacial, infra-orbital, auriculotemporal, buccal, mental, great auricular, lesser occipital, greater occipital, third occipital, erbs point, supraclavicular, lateral supraclavicular and intermediate supraclavicular.

Beneficial Effects and Synergy

Various components that can be used in the therapeutic compositions disclosed herein can have beneficial and synergistic affects. D-Limonene (and other Terpenes) can have a threefold effect: 1) Enhanced analgesic properties of other analgesic components of the topical compound; 2) Improved delivery of therapeutic medium across the skin; and 3) Neutralized menthol smell and sensation (e.g., no burning, stinging).

Zinc or zinc oxide, can have multiple effects that improve performance of topical compound, such as analgesic properties, facilitating electrical transmission (e.g., enhanced the effect of electrical stimulation of nerves while allowed lower intensities of stimulation, which improved comfort and enhanced analgesic effect), antimicrobial effect (e.g., without being harsh on the skin); and enhancing anti-comedogenic effects.

Pterostilbene can have analgesic properties, can be synergistic with sugar to enhance analgesia, can limits microbial growth, which can prolong shelf life and microbial resistance of formula.

The use of fruit, plant or seed extracts was found to be a stable way of delivering TOR-, mTOR-modulators and polyphenols.

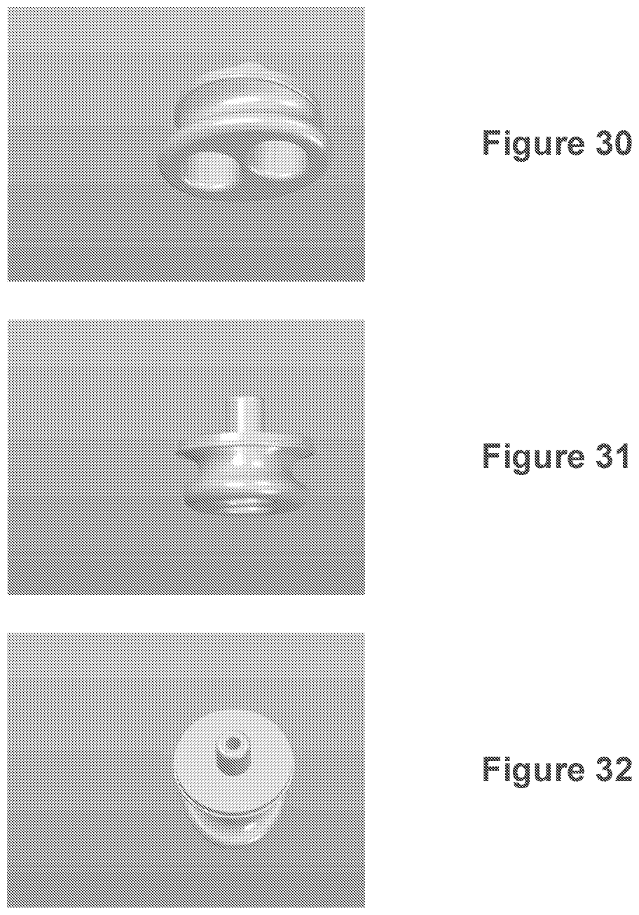

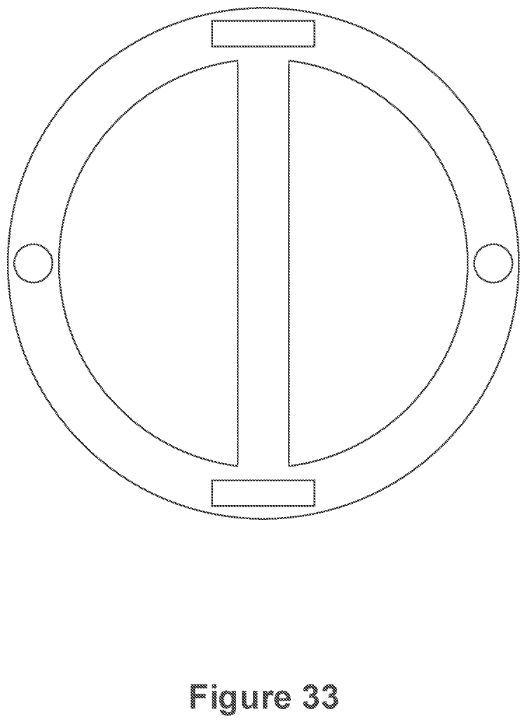

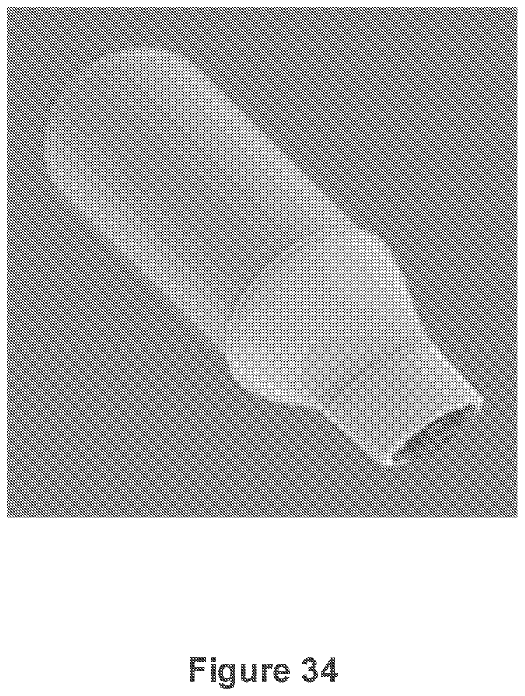

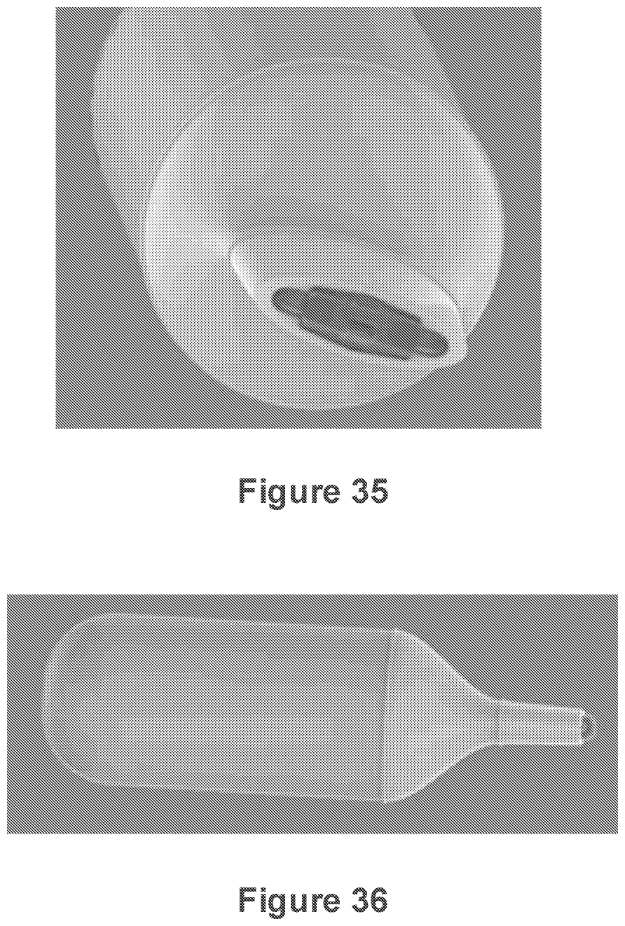

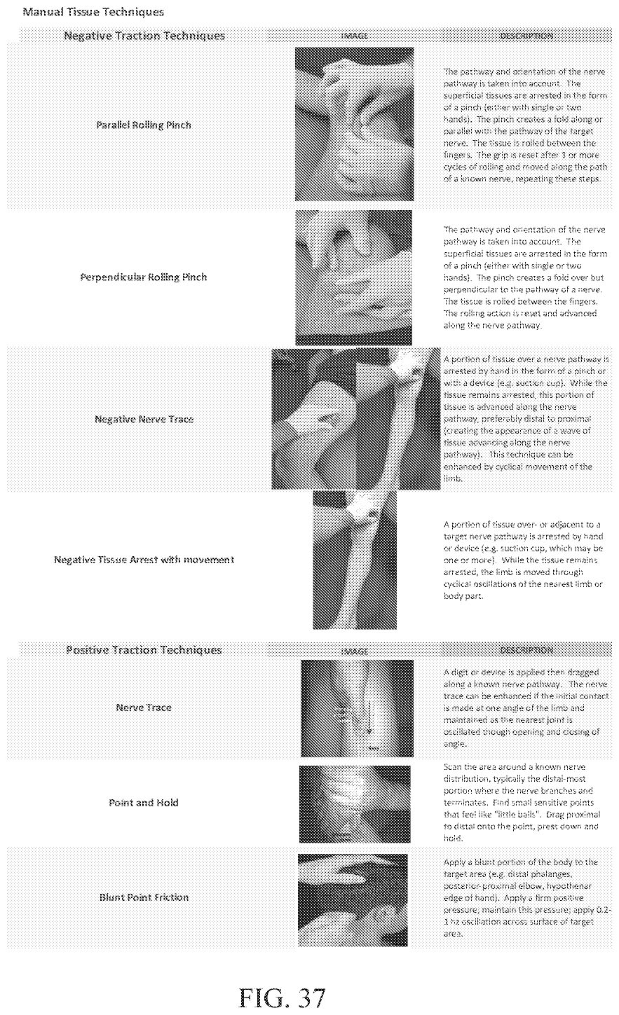

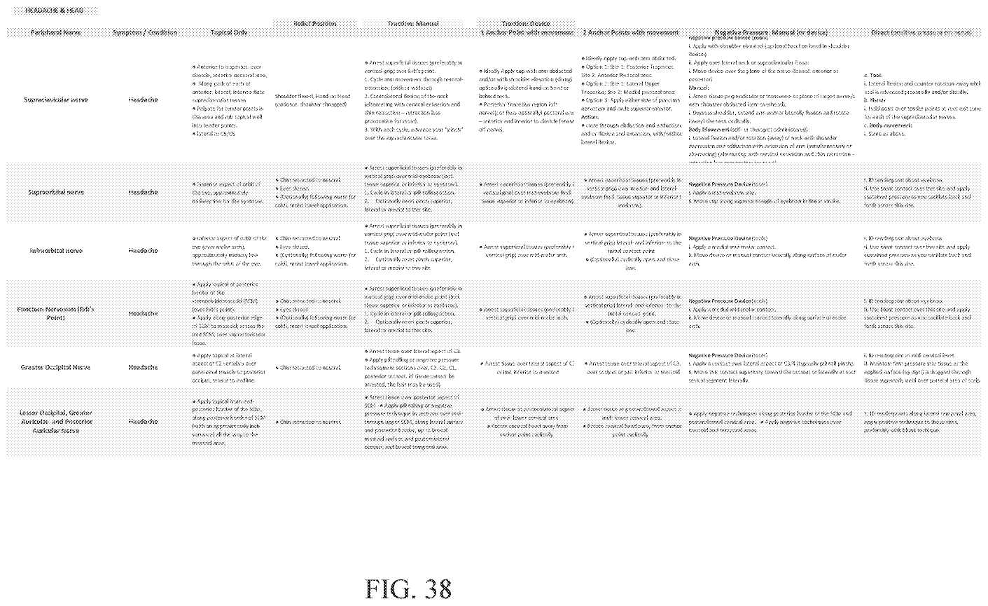

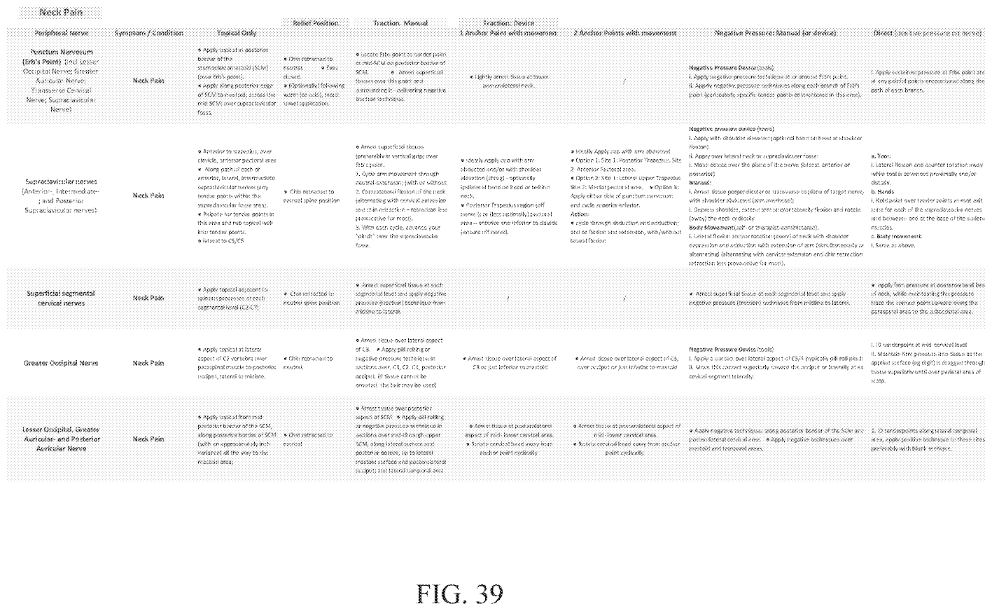

Multiple (e.g., 2 more more) adjacent chambers in pressure therapy device interface (e.g., FIGS. 30-33) with skin can have the benefits of being more effective than single compartment because it combines negative pressure with positive pressure (e.g., delivered by dividing wall between the chambers). These embodiments can create a “two camel-hump” effect where two sections of tissue are drawn up by negative pressure while the divider wall acts as friction tool (e.g., without actually depressing the tissue), as the device advances over section of skin, which can be a more comfortable manner of delivering friction and more effective that direct positive pressure.

A manual or device technique that draws tissue (e.g, vacuum, traction, pulling etc.) to mobilize tissue surrounding nerves is effective way to reduce nerve symptoms (including pain), which can be markedly enhanced by the simultaneous application of a sugar/sugar alcohol-based compound, as described herein, or a combination of sugar and sugar-alcohol in the compound (e.g., less digestive concerns in sensitive individuals). Analgesic effect of mannitol can be preserved with more widely available sugar form Plant- or fruit-extracts can be used in the composition, as discussed herein. Cosmetics can contain plant- or fruit-extracts applied over superficial nerves of face and head to reduce symptoms of headache.

Additional Information

In some embodiments, the methods, techniques, microprocessors, and/or controllers described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination thereof. The instructions can reside in RAM memory, flash memory, ROM memory, EPROM memory, EEPROM memory, registers, hard disk, a removable disk, a CD-ROM, or any other form of a non-transitory computer-readable storage medium. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

The microprocessors or controllers described herein can be coordinated by operating system software, such as iOS, Android, Chrome OS, Windows XP, Windows Vista, Windows 7, Windows 8, Windows 10, Windows Server, Windows CE, Unix, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

The microprocessors and/or controllers described herein may implement the techniques described herein using customized hard-wired logic, one or more ASICs or FPGAs, firmware and/or program logic which causes microprocessors and/or controllers to be a special-purpose machine. According to one embodiment, parts of the techniques disclosed herein are performed a controller in response to executing one or more sequences instructions contained in a memory. Such instructions may be read into the memory from another storage medium, such as storage device. Execution of the sequences of instructions contained in the memory causes the processor or controller to perform the process steps described herein. In alternative embodiments, hard-wired circuitry may be used in place of or in combination with software instructions.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the techniques described herein may be implemented in analog circuitry or mixed analog and digital circuitry.

Unless the context clearly requires otherwise, throughout the description and the claims, the words “comprise,” “comprising,” “include,” “including,” and the like are to be construed in an inclusive sense, as opposed to an exclusive or exhaustive sense; that is to say, in the sense of “including, but not limited to.” The words “coupled” or connected,” as generally used herein, refer to two or more elements that can be either directly connected, or connected by way of one or more intermediate elements. Additionally, the words “herein,” “above,” “below,” and words of similar import, when used in this application, shall refer to this application as a whole and not to any particular portions of this application. Where the context permits, words in the Detailed Description using the singular or plural number can also include the plural or singular number, respectively. The words “or” in reference to a list of two or more items, is intended to cover all of the following interpretations of the word: any of the items in the list, all of the items in the list, and any combination of the items in the list. All numerical values provided herein are intended to include similar values within a range of measurement error.

Although this disclosure contains certain embodiments and examples, it will be understood by those skilled in the art that the scope extends beyond the specifically disclosed embodiments to other alternative embodiments and/or uses and obvious modifications and equivalents thereof. In addition, while several variations of the embodiments have been shown and described in detail, other modifications will be readily apparent to those of skill in the art based upon this disclosure. It is also contemplated that various combinations or sub-combinations of the specific features and aspects of the embodiments may be made and still fall within the scope of this disclosure. It should be understood that various features and aspects of the disclosed embodiments can be combined with, or substituted for, one another in order to form varying modes of the embodiments. Any methods disclosed herein need not be performed in the order recited. Thus, it is intended that the scope should not be limited by the particular embodiments described above.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. Any headings used herein are for the convenience of the reader only and are not meant to limit the scope.

Further, while the devices, systems, and methods described herein may be susceptible to various modifications and alternative forms, specific examples thereof have been shown in the drawings and are herein described in detail. It should be understood, however, that the disclosure is not to be limited to the particular forms or methods disclosed, but, to the contrary, this disclosure covers all modifications, equivalents, and alternatives falling within the spirit and scope of the various implementations described. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication.

The ranges disclosed herein also encompass any and all overlap, sub-ranges, and combinations thereof. Language such as “up to,” “at least.” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including ambient temperature and pressure.

1. A method of treating neurogenic pain, the method comprising:
accessing an applicator that includes a housing that defines an internal cavity, with a therapeutic composition inside the internal cavity;
dispensing an amount of the therapeutic composition from the internal cavity onto a contact tip that is coupled to the housing; and
pressing the contact tip against skin at a nerve location or along a nerve pathway that is associated with the neurogenic pain so that the therapeutic composition is administered transdermally.

2.-105. (canceled)
